(19) United States
(12) Patent Application Publication
(10) Pub. No.: US 2021/0254186 A1
(43) Pub. Date: Aug. 19, 2021

(54) FLAVIVIRUS DIAGNOSTIC ARRAY

(71) Applicant: The Secretary of State for Health, London (GB)

(72) Inventors: Robert Watson, Salisbury (GB); Laura Bonney, Salisbury (GB)

(73) Assignee: The Secretary of State for Health, London (GB)

(21) Appl. No.: 16/324,107

(22) PCT Filed: Aug. 7, 2017
(86) PCT No.: PCT/GB2017/052320

§ 371 Date: Feb. 7, 2019

(30) Foreign Application Priority Data
Aug. 8, 2016 (GB) 1613601.2

(51) Int. Cl. C12Q 1/70

(52) U.S. Cl. CPC C12Q 1/701

(57) ABSTRACT

The present invention provides nucleic acid products and corresponding methods for identifying the presence or absence of Zika virus in a sample.

The invention relates to nucleic acid products and to the detection of Zika virus. More specifically, the invention relates to new primers and probes for detecting the presence of Zika virus in a sample.

Zika virus is a mosquito-borne virus of the Flavivirus genus, and is closely related to Dengue virus. Although traditionally associated with mild febrile illness, Zika virus has recently been linked with more serious conditions including Guillain-Barre syndrome and foetal microcephaly. Serological surveys have demonstrated a rapid spread of Zika virus within recent years from its origins in South-East Asia, with over 50 countries currently reporting active Zika virus transmission (based on Centres for Disease Control and Prevention statistics). There is significant potential for further spread of Zika virus, due to the multi-species animal reservoir, potential for the virus to cross to additional mosquito vectors (with lab tests showing viable carriage by the Aedes albopictus mosquito common in a large part of the world) and other modes of transmission including sexual and transfusion-based transfer.

In response to the current Zika virus outbreak, the European Centre for Disease Prevention and Control (ECDC) is advocating an increase in Zika testing laboratory capacity in the EU, the Brazilian government has promised a large capital investment to promote new Zika diagnostic development and the World Health Organisation (WHO) has declared the outbreak a public health emergency of international concern. There is therefore an urgent and rapidly expanding demand for diagnostics for Zika virus.

There are, at present, no FDA-approved diagnostics for identification of Zika virus, but several diagnostic techniques have been authorised temporarily by the FDA under an Emergency Use Authorisation (EUA), for use during the emergency response. Most of the current Zika virus diagnostic assays are RT-PCR based (RealStar® Zika Virus RT-PCR Kit (Altona Diagnostics), Zika Virus RNA Qualitative Real-Time RT-PCR (Focus Diagnostics) and the Trioplex Real-time PT-PCR Assay (CDC).

Whilst existing RT-PCR based methods can provide high levels of sensitivity (with some detecting down to a single genome copy), these methods are hampered by significant limitations. For example, existing RT-PCR based methods are slow (typically 1 hour run-time on a standard RT-PCR machine), and are therefore not ideally-suited to high-throughput screening.

Moreover, existing RT-PCR based methods have a high power requirement, which is typically accommodated by a mains electrical source. This power-requirement significantly reduces portability of these methods, and can severely limit their suitability for use at the point-of-care (POC) which, in the context of Zika virus detection, is often in remote rural locations. Thermocyclers are also expensive, which further limits their suitability for widespread use.

Existing RT-PCR based methods also require intensive sample processing work, and are typically highly sensitive to sample quality. This increases sample preparation time and complexity, and typically requires the involvement of skilled technicians.

In view of the various limitations with existing RT-PCR based detection methods, there is significant interest in developing serological assays for detecting Zika virus. These antibody-based detection assays for Zika virus are advantageous because they are relatively simple to perform, and provide a readout that is easy to understand. However, they are suffer from serious cross-reactivity with related viral strains (such as the endemic Dengue virus, West Nile virus etc.), or previous immunisation (e.g. yellow fever vaccination), which creates major challenges in positively identifying Zika virus, and discriminating between viral strains. Moreover, these serological methods are unable to detect early infection or determine viral loads, and they are less sensitive than existing RT-PCR based assays. IgM-based serological methods can detect active-infection, but are hampered by cross-reactivity problems.

The only isothermal detection method currently authorised under the EUA is the Aptima® Zika Virus assay (Hologic.Inc.), which is a Transcription Mediated Amplification (TMA)-based method. When used in conjunction with the laboratory-based Panther system for automated specimen processing, this method is highly sensitive, detecting down to 10-30 copies/ml original starting material. However, this method is limited by an extremely slow (3.5 hour) turnaround time. Moreover, this method requires an expensive laboratory-restricted automated specimen processing system, which severely limits its portability, and is thus restricted to use in centralised laboratories.

There is therefore an urgent and unmet need for a rapid, simple and fieldable technology, that would enable reliable identification of Zika virus.

The present invention solves one or more of the above-identified problems by providing a flexible, accurate, rapid, robust and sensitive method for detecting Zika virus.

Specifically, the invention provides a method for detecting Zika virus in a sample, via detection of Zika virus nucleic acid in the sample. The terms “detecting Zika virus in a sample” and “detecting Zika virus nucleic acid in a sample” (and the like) are, within the context of the invention, used interchangeably.

According to the invention, methods for detecting Zika virus in a sample typically comprise amplifying the amount of Zika virus nucleic acid in the sample. In such cases, the skilled person will appreciate that “detection of Zika virus nucleic acid in the sample” typically involves detection of Zika virus nucleic acid amplicon and/or the corresponding amplification product. In one embodiment, said amplification product comprises a fluorescent moiety, and the detection of Zika virus nucleic acid in the sample comprises detection of said fluorescent moiety.

Zika virus is an RNA virus, and so detection of Zika virus typically comprises detection of Zika virus RNA (or e.g. nucleic acid derived from Zika virus RNA).

The primers and probes of the invention are highly suitable for use in the detection of Zika virus in remote rural settings, as well as peripheral hospital sites (where there is greater access to acute samples of serum, saliva and urine), and the inventors believe that the invention will revolutionise the global response to Zika virus.

Accordingly, the invention provides a composition comprising: a nucleic acid probe comprising: (i) nucleic acid sequence of any one of SEQ ID NOs: 31-114; or (ii) nucleic acid sequence exhibiting at least 85% identity to any one of SEQ ID NOs: 31-114; or (iii) nucleic acid sequence comprising 15 or more consecutive nucleic acids of the nucleic acid sequence of SEQ ID NO: 31-114; wherein group “3”=modification that functions to block polymerase extension; “5”=dT-fluorophore; “6”=an abasic nucleotide analog; and “7”=dT-quencher group (suitable for group “5”); and/or b) a forward nucleic acid primer and a reverse nucleic acid primer; the forward nucleic acid primer comprising: i) nucleic acid sequence GTGAAGCCTACCTTG (SEQ ID NO: 2); or ii) nucleic acid sequence exhibiting at least 85% identity to the nucleic acid sequence of SEQ ID NO: 2; and the reverse nucleic acid primer comprising: i) nucleic acid sequence TGGATGCTCTTCCCG (SEQ ID NO: 17); or ii) nucleic acid sequence exhibiting at least 85% identity to the nucleic acid sequence of SEQ ID NO: 17.

In one embodiment, group “3” is selected from (i) C₃-spacer, (ii) a phosphate, (iii) a biotin-TEG, or (iv) an amine; group “5” is selected from (i) dT-fluorescein, (ii) TAMRA and (iii) Cy5; group “6” is D-spacer; and group “7” is selected from (i) dT-BHQ1 and (ii) dT-BHQ2. In one embodiment, group “3” is propanol; group “5” is dT-fluorescein; group “6” is D-spacer; and group “7” is dT-BHQ1.

In one embodiment, the forward nucleic acid primer comprises or consists of (i) the nucleic acid sequence of any one of SEQ ID NOs: 1-15; or (ii) nucleic acid sequence exhibiting at least 85% identity to the nucleic acid sequence of any one of SEQ ID NOs: 1-15; or (iii) nucleic acid sequence comprising 15 or more consecutive nucleic acids of the nucleic acid sequence of SEQ ID NO: 1 or any one of SEQ ID NOs: 1-15.

In one embodiment, the reverse nucleic acid primer comprises or consists of: (i) the nucleic acid sequence of or any one of SEQ ID NOs: 16-30; or (ii) nucleic acid sequence exhibiting at least 85% identity to the nucleic acid sequence of any one of SEQ ID NOs: 16-30; or (iii) nucleic acid sequence comprising 15 or more consecutive nucleic acids of the nucleic acid sequence of any one of SEQ ID NOs: 16-30.

In one embodiment, the composition comprises: (i) forward nucleic acid primer comprising or consisting of the nucleic acid sequence of SEQ ID NO: 3, and/or exhibiting at least 85% identity to the nucleic acid sequence of any one of SEQ ID NO: 3; (ii) reverse nucleic acid primer comprising or consisting of the nucleic acid sequence of SEQ ID NO: 18, and/or exhibiting at least 85% identity to the nucleic acid sequence of any one of SEQ ID NO: 18; and (iii) nucleic acid probe comprising or consisting of the nucleic acid sequence of SEQ ID NO: 31, and/or exhibiting at least 85% identity to the nucleic acid sequence of any one of SEQ ID NO: 31.

In one embodiment, the composition comprises: (i) forward nucleic acid primer comprising or consisting of the nucleic acid sequence of SEQ ID NO: 1, and/or exhibiting at least 85% identity to the nucleic acid sequence of any one of SEQ ID NO: 1; (ii) reverse nucleic acid primer comprising or consisting of the nucleic acid sequence of SEQ ID NO: 16, and/or exhibiting at least 85% identity to the nucleic acid sequence of any one of SEQ ID NO: 16; and (iii) nucleic acid probe comprising or consisting of the nucleic acid sequence of SEQ ID NO: 31, and/or exhibiting at least 85% identity to the nucleic acid sequence of any one of SEQ ID NO: 31.

The invention also provides a kit comprising a composition as described above.

The invention also provided a device comprising a composition as described above.

In one embodiment, the kit or the device comprises part or whole of a TwistAmp Basic kit, TwistAmp Basic-RT kit, TwistAmp exo kit, TwistAmp exo-RT kit, TwistAmp fpg kit, and/or TwistAmp nfo kit.

The invention also provides use of a composition, kit or device as described above, in a method of detecting Zika virus in a sample.

The invention also provides a method for detecting the presence of Zika virus in a sample or detecting the absence of said Zika virus in said sample, said method comprising: A) combining said sample with a composition as described above; B) allowing nucleic acid present in the sample to contact the primers and/or probes within the composition; and C) performing a nucleic acid amplification technique; wherein amplification of nucleic acid in the sample confirms that nucleic acid from Zika virus is present within the sample, and wherein the absence of amplification of nucleic acid in the sample confirms that nucleic acid from Zika virus is absent from the sample. In one embodiment, the nucleic acid amplification technique is an isothermal nucleic acid amplification technique, such as Recombinase Polymerase Amplification (RPA).

In one embodiment, the sample is from an individual, typically an animal, typically a human. In such an embodiment, the sample is typically selected from blood, plasma, saliva, serum, sputum, urine, cerebral spinal fluid, semen, cells, a cellular extract, a tissue sample, a tissue biopsy, a stool sample, a swab from any body site and/or one or more organs; typically blood, serum, urine, saliva and/or organ(s).

In one embodiment, the animal is an insect, typically a mosquito, typically an Aedes mosquito. In such an embodiment, the sample is typically homogenised mosquito(s).

In one embodiment, the sample is a crude sample.

In one embodiment, the method is for use in surveillance of Zika virus prevalence.

In one embodiment, the method is for use in diagnosing Zika virus infection in an individual. In one embodiment, upon identification of Zika virus infection in the individual, said individual is provided with an appropriate treatment or therapy.

There now follows a brief description of the Figures, which illustrate embodiments of the present invention.

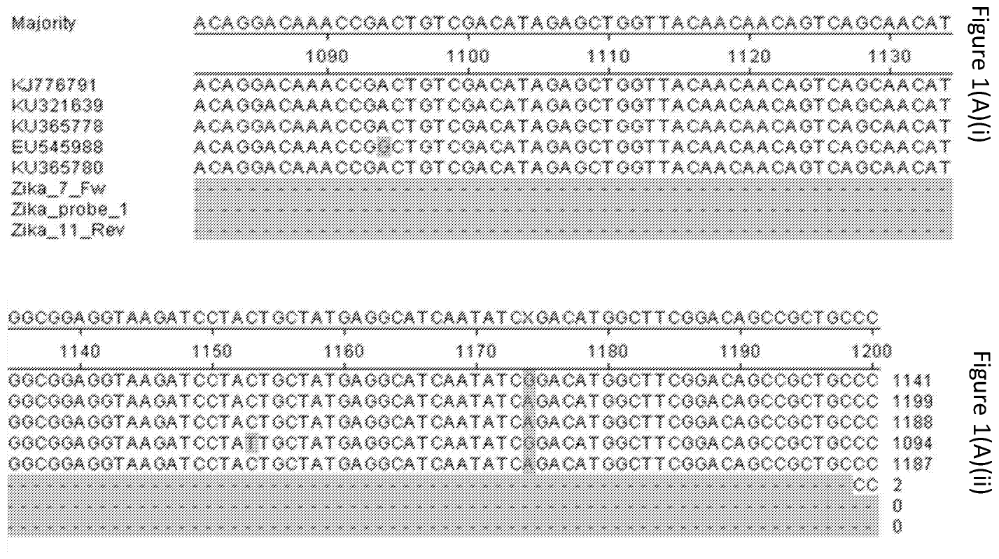

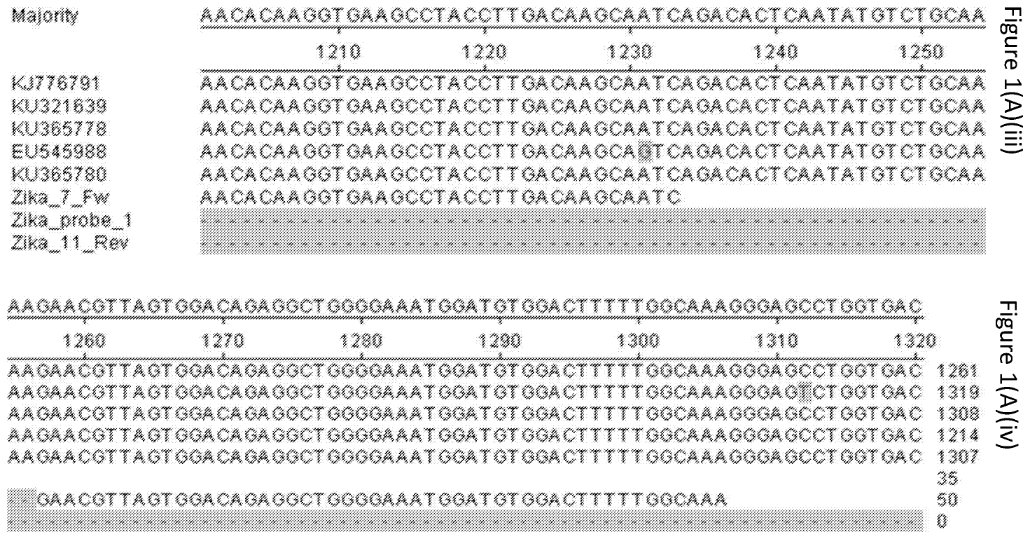

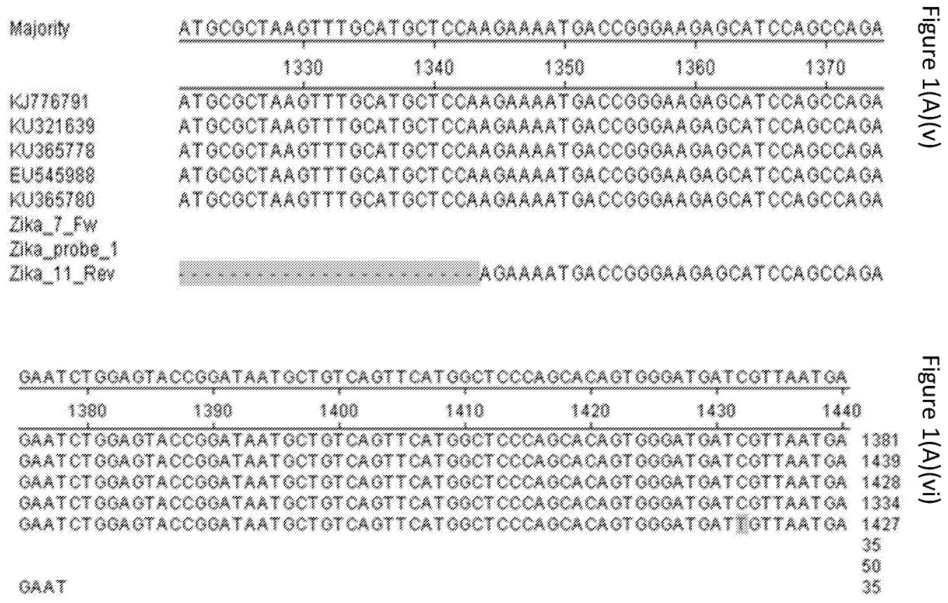

FIG. 1: Design of primers and probes of the invention. FIG. 1(A) shows an alignment of synthetic RNA template and extracted cultured Zika virus with “Zika RPA 7 Fw” (forward primer; identified in FIG. 1(A) as “Zika_7_Fw”), “Zika RPA 11 Rev” (reverse primer; identified in FIG. 1(A) as “Zika_11_Fw”) and “Zika RPA Probe 1” (probe; identified in FIG. 1(A) as “Zika_probe_1”). FIG. 1(B) shows an alignment of synthetic RNA template and extracted cultured Zika virus with “Zika RPA Fw” (forward primer; identified in FIG. 1(B) as “Zika_RPA Fw”), “Zika RPA Rev” (reverse primer; identified in FIG. 1(B) as “Zika_RPA Rev) and “Zika RPA Probe 1” (probe; identified in FIG. 1(B) as “Zika RPA_probe”).

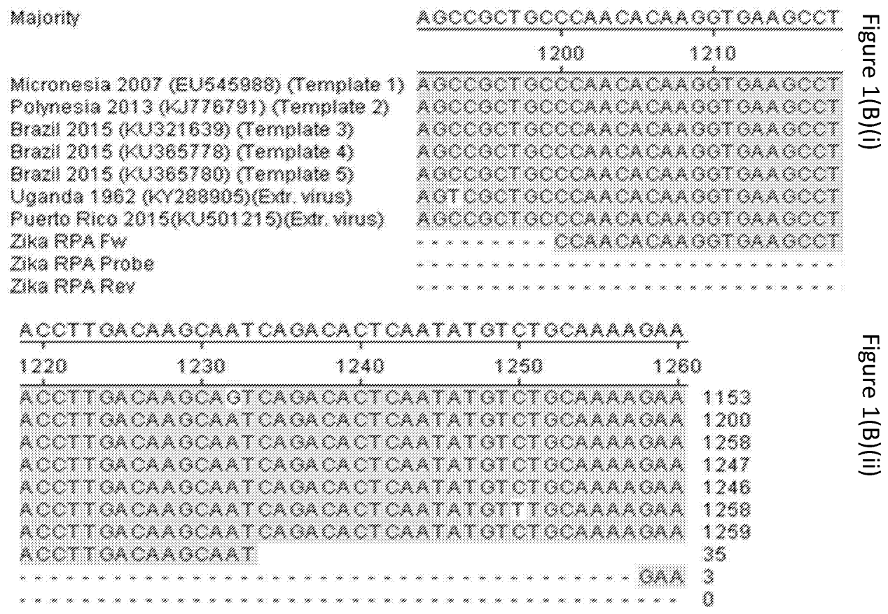

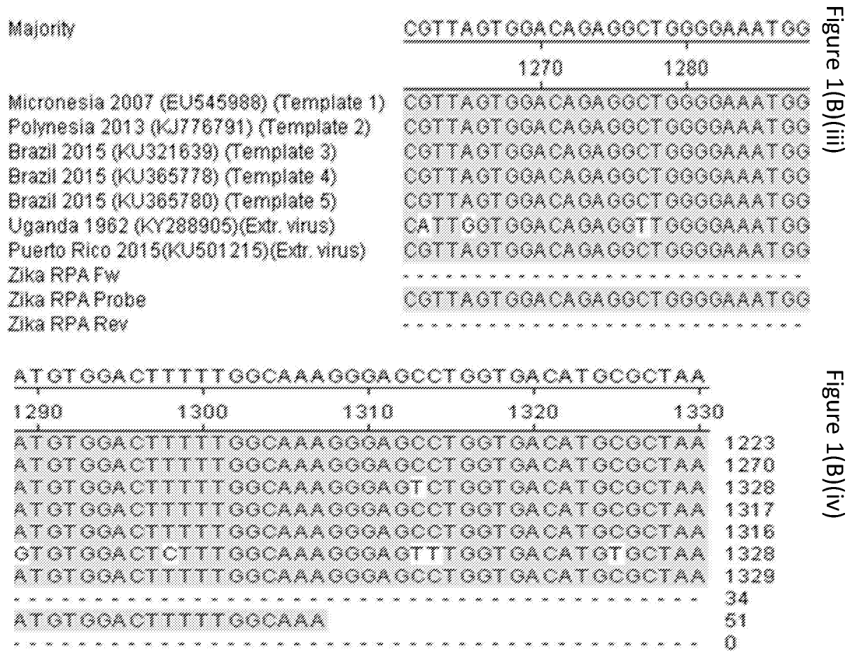

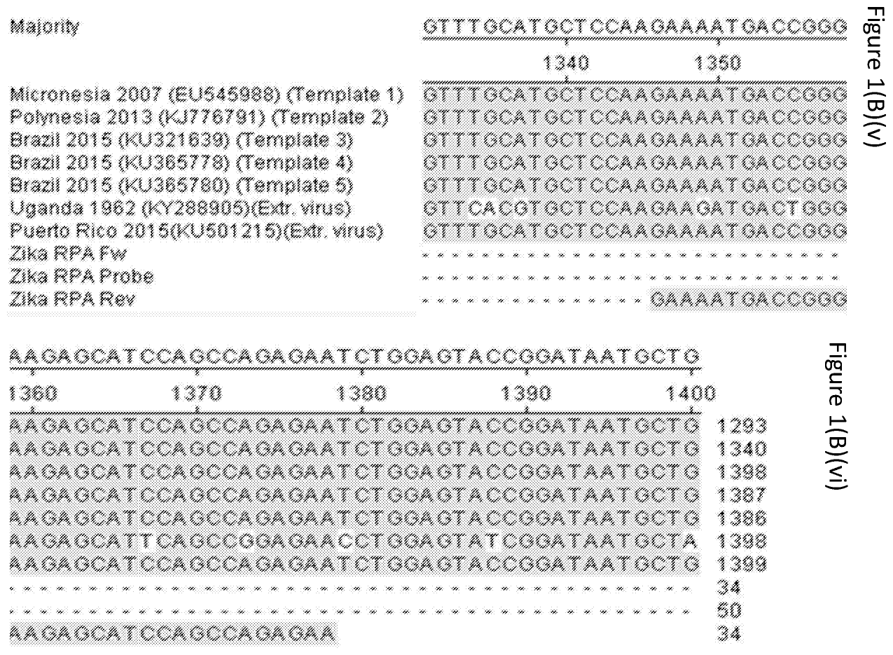

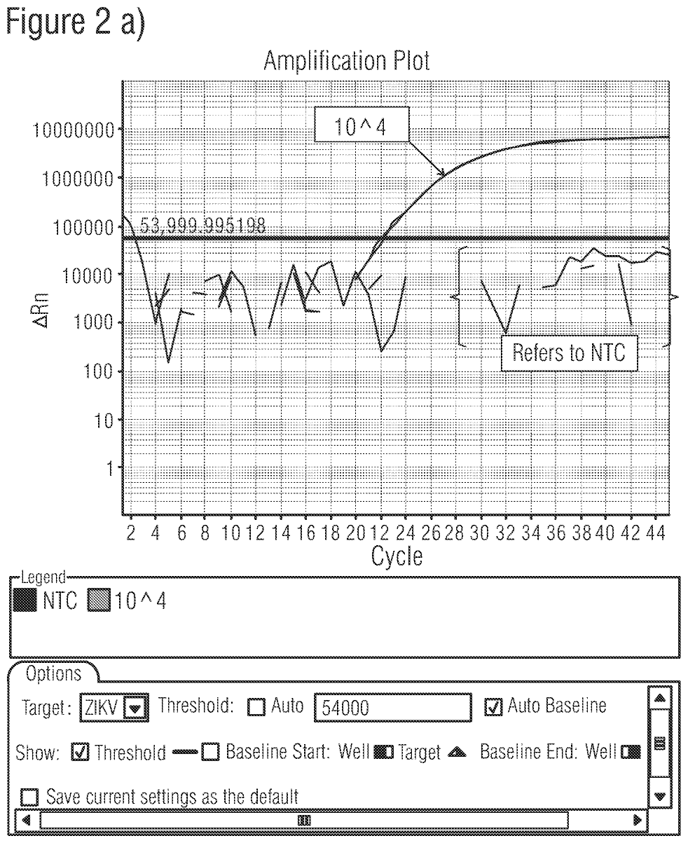

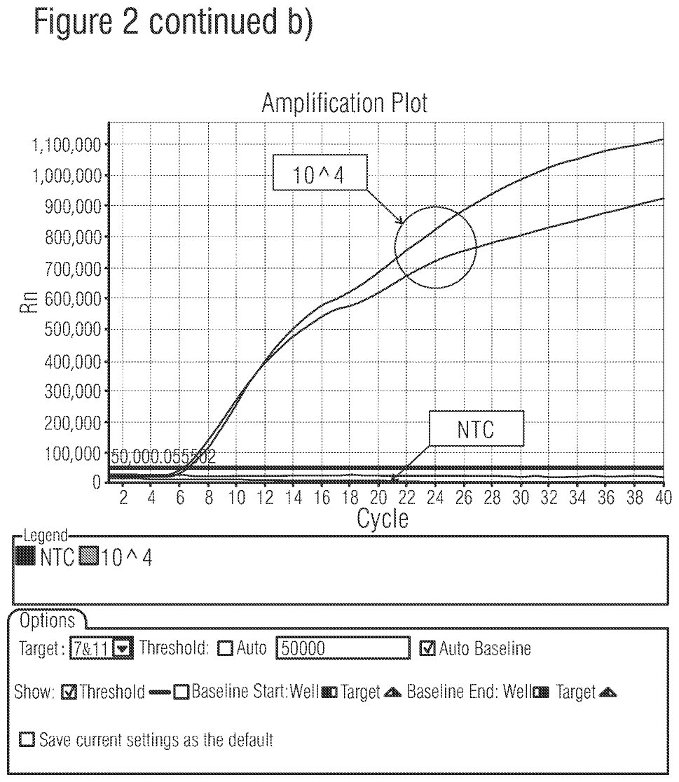

FIG. 2: Time to positive with a South American outbreak strain (synthetic Zika RNA template used at 50,000 copies of target/reaction). Panel a) shows results from E-gene PCR ((Adapted from the method published by Lanciotti et al. Genetic and Serologic Properties of Zika Virus Associated with an Epidemic, Yap State, Micronesia, 2007. Emerg Infect Dis. 2008 August; 14(8): 1232-1239); and panel b) shows results obtained using an RPA assay of the invention. NTC=no template control.

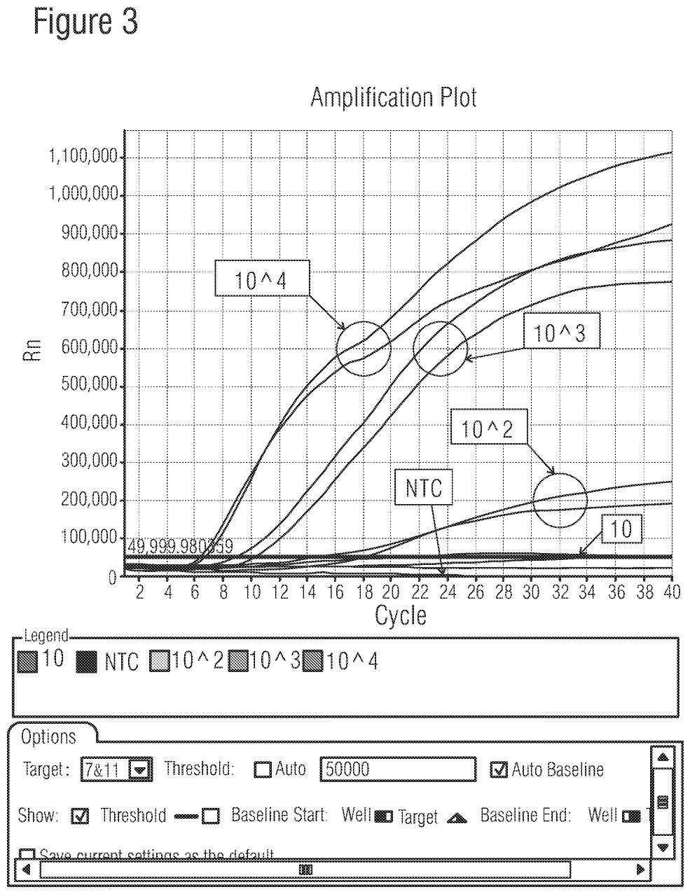

FIG. 3: RPA limit of detection with a synthetic Zika RNA template (South American outbreak strain).

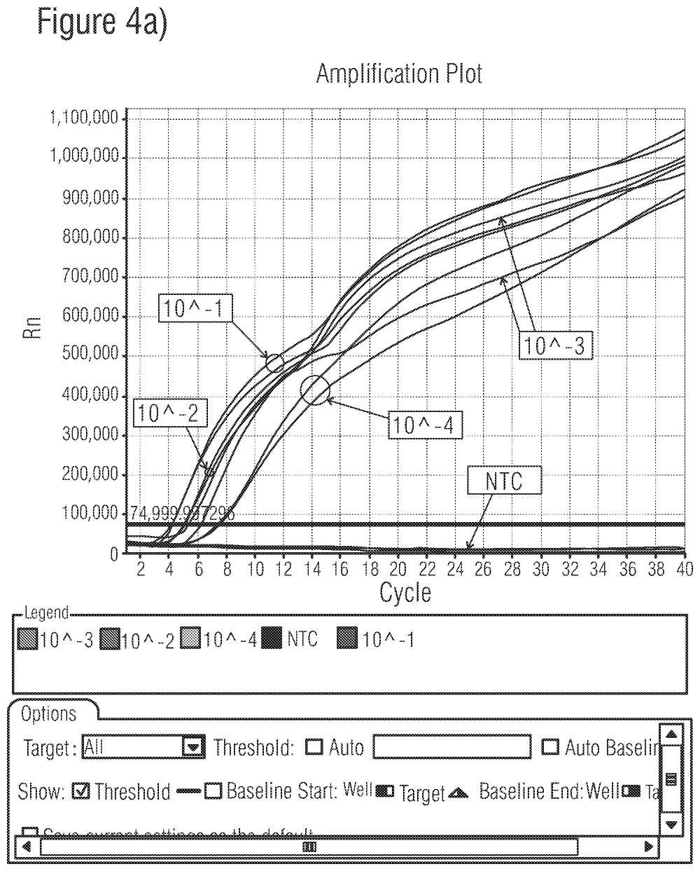

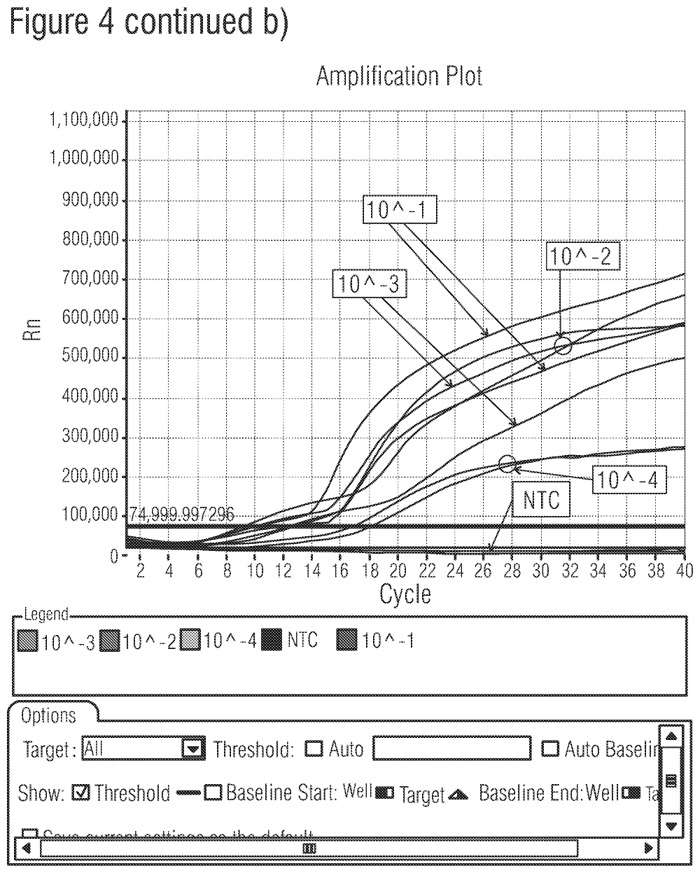

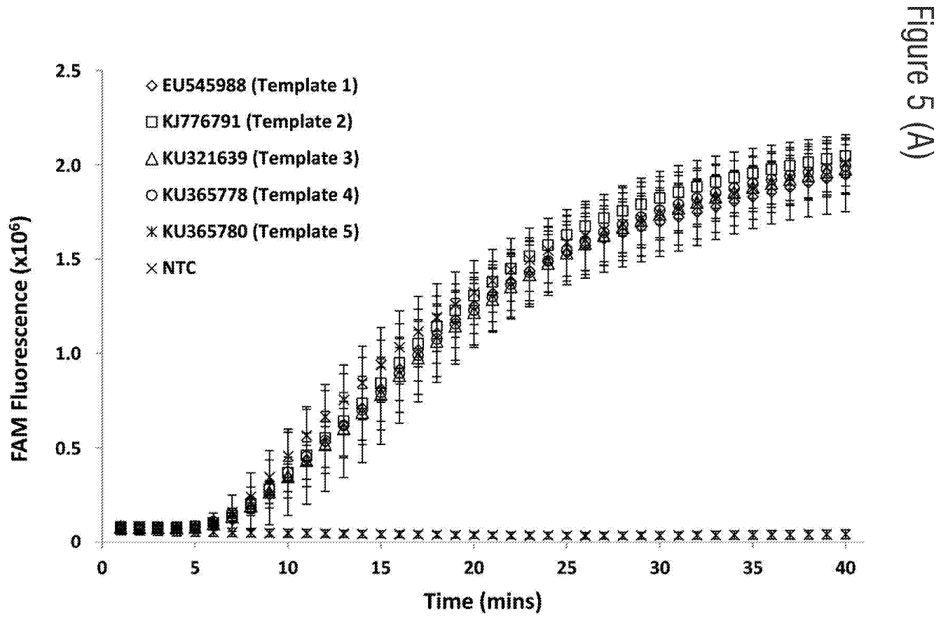

FIG. 4: RPA assay performed using different Zika virus extracts. Panel a) shows results obtained using South American outbreak strain (CDC); and panel b) shows results obtained using pre-outbreak African strain (NCPV).

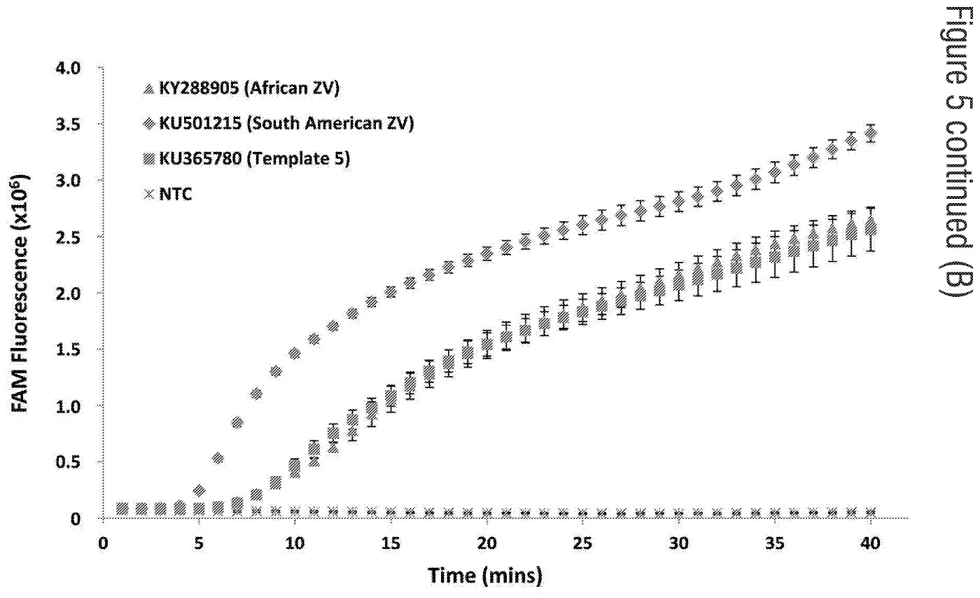

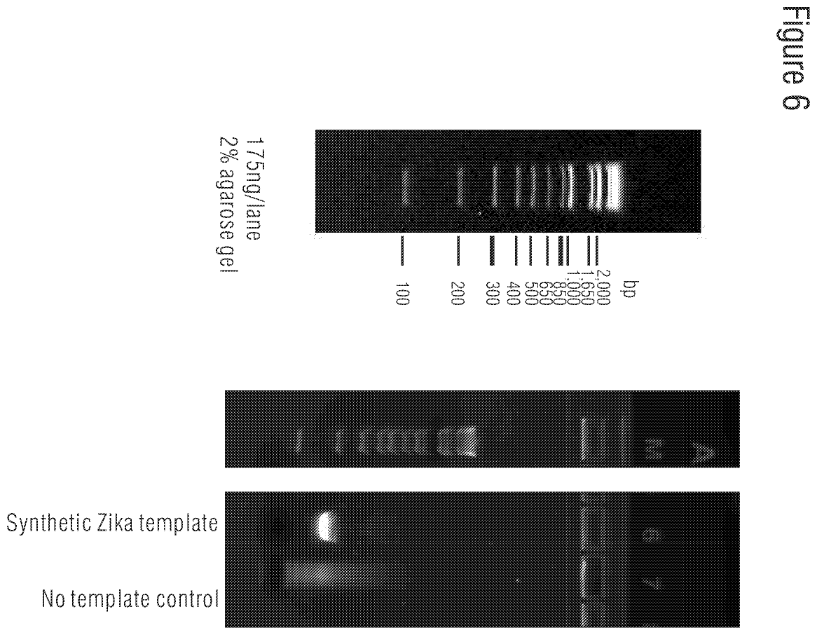

FIG. 5: RPA assay performed using different Zika templates (1-5). Panel a) shows results using different Zika synthetic RNA templates. All synthetic fragments were used at 5×10³ copies per reaction and compared to the non-template control (NTC). Panel b) shows amplification of target region using extracted nucleic acid from two strains of cultured Zika virus (African—KY288905 and South American—KU501215) and compared to detection of Zika synthetic RNA fragment 5 (KU365780). Both cultured viral strains were used in the assay at 1.5×10¹ PFU per reaction, whereas the synthetic fragment 5 was used at 5×10³ copies per reaction. Fluorescent signal generated by the non-template control (NTC) is also shown in reference.

FIG. 6: Gel electrophoresis showing reaction product from RPA assay (amplified DNA) performed using primers and probe of the invention. Performed using a basic RPA kit and a synthetic template (South American outbreak strain).

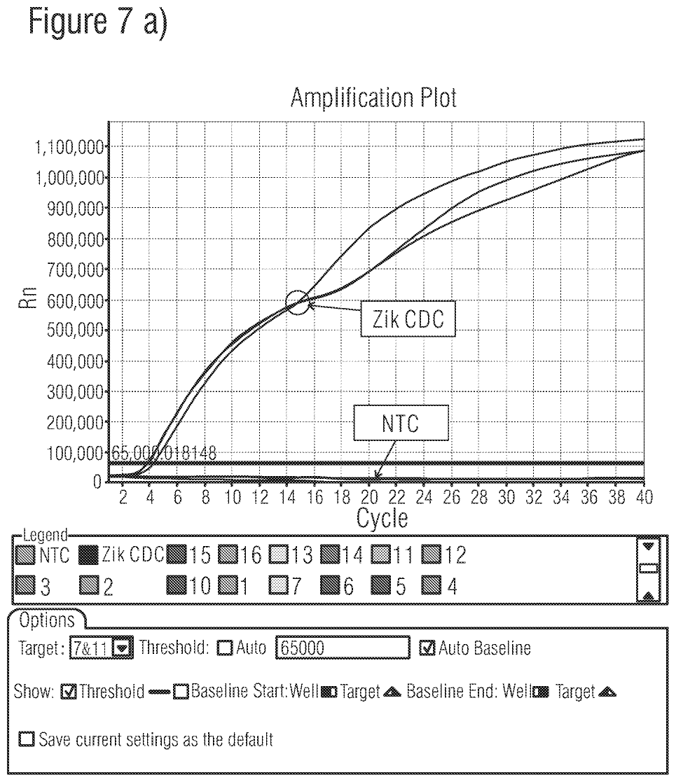

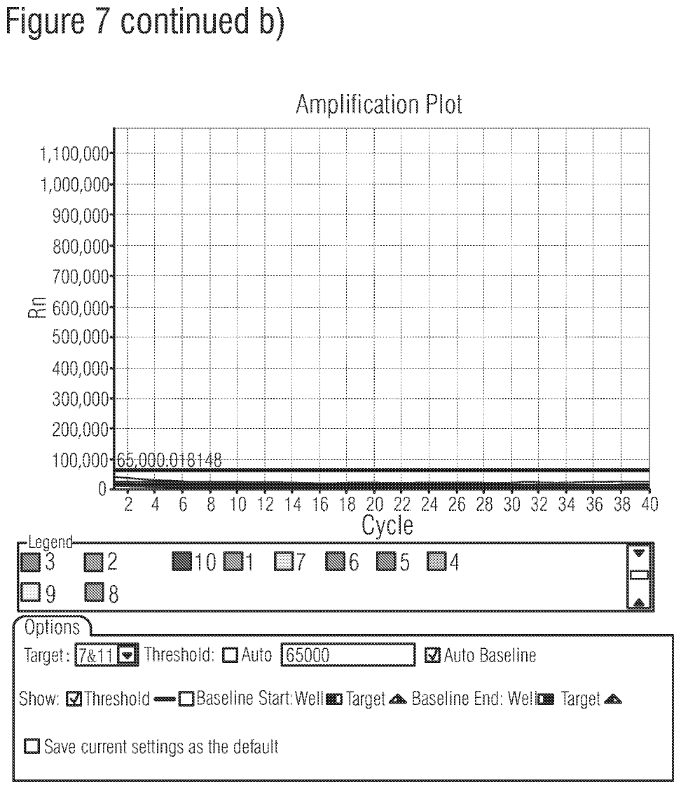

FIG. 7: Negative panel testing with a selection of viral extracts. Panel a) shows results obtained using assay controls; and panel b) shows results obtained using negative viral panel.

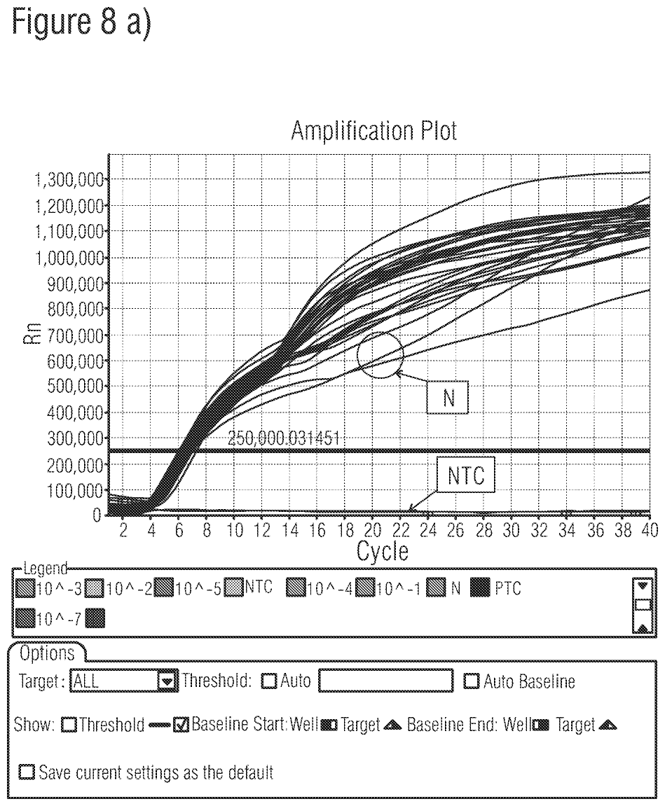

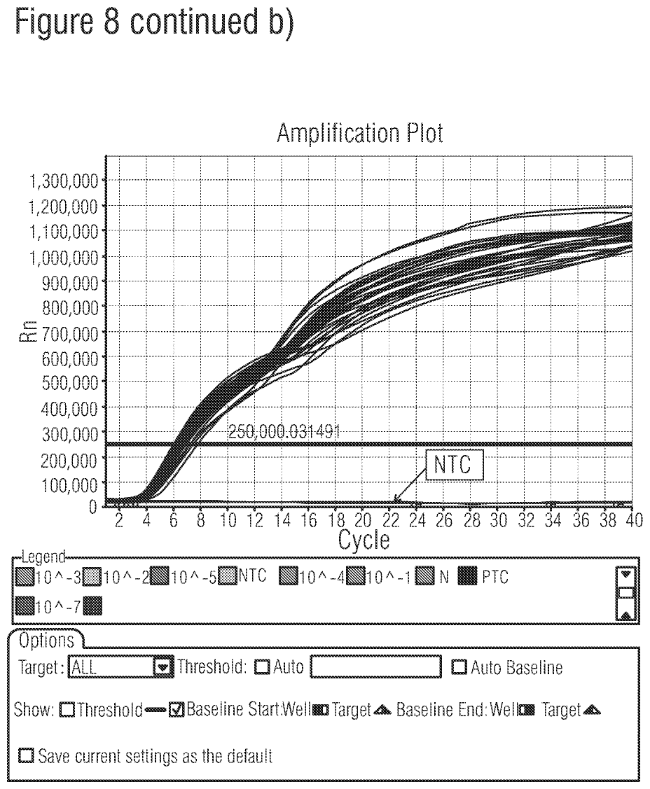

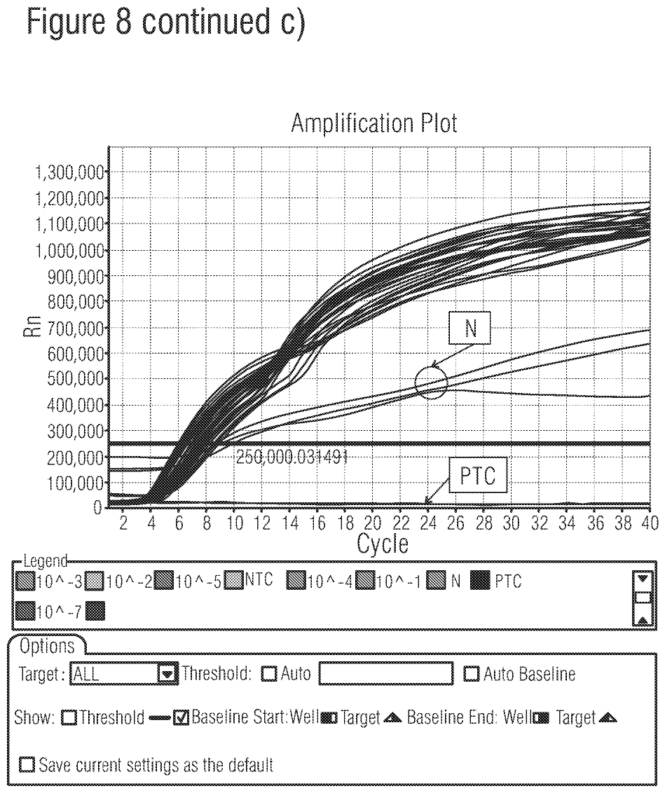

FIG. 8: Inhibitory effects of crude samples on the tested RPA assay. Panel a) results obtained in the presence of serum; panel b) results obtained in the presence of urine standard; and panel c) results obtained in the presence of mosquito larvae homogenate.

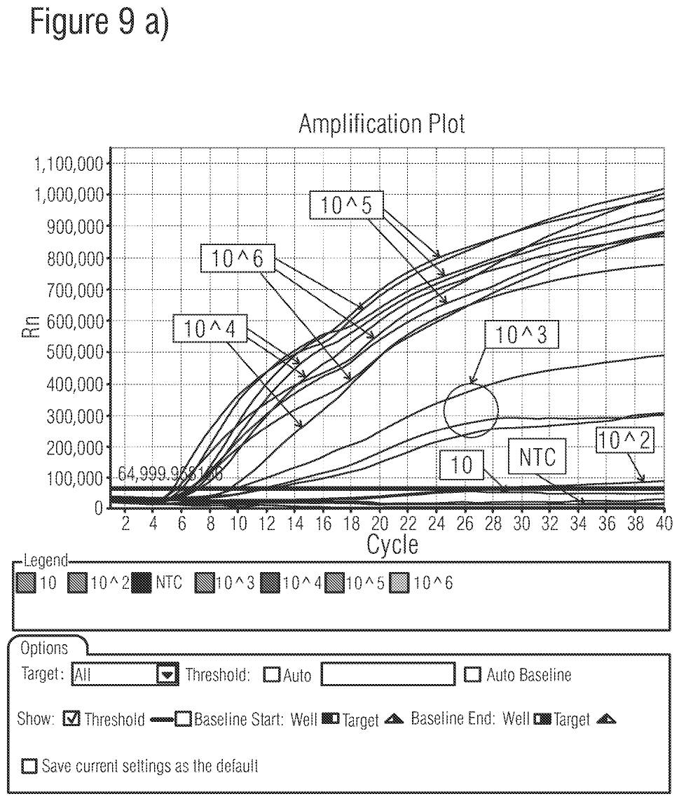

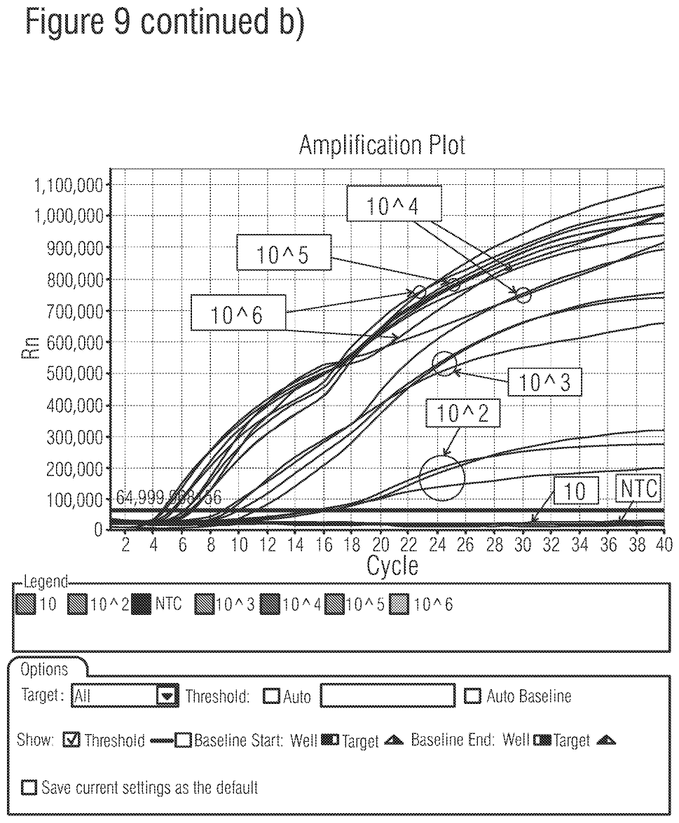

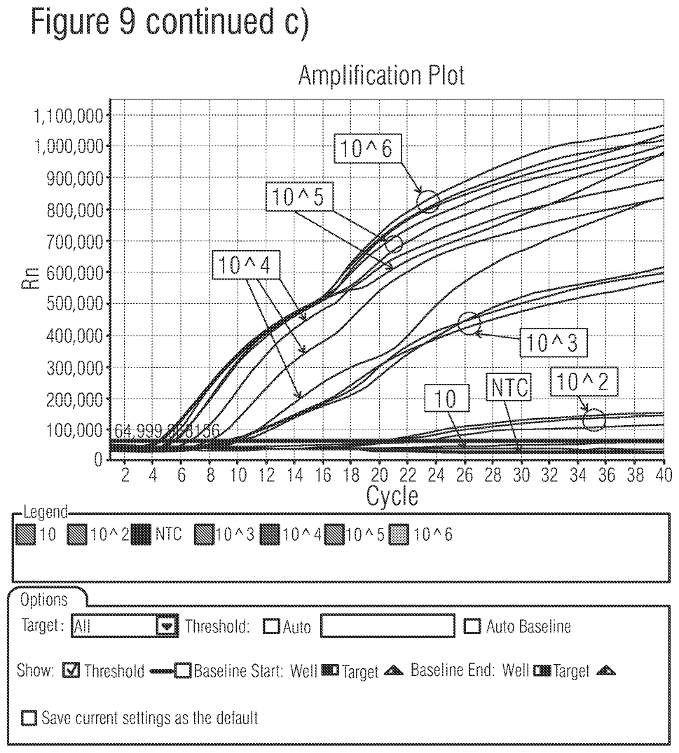

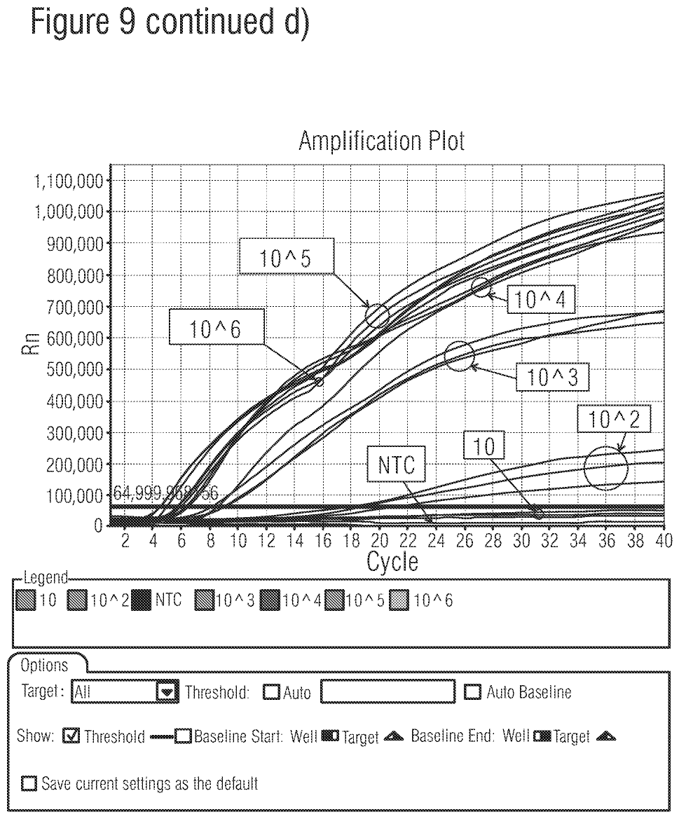

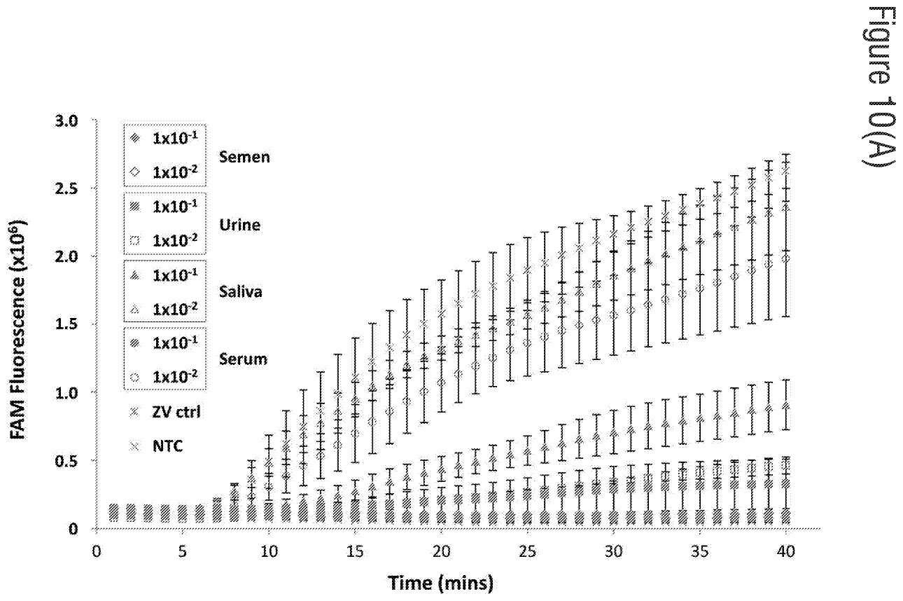

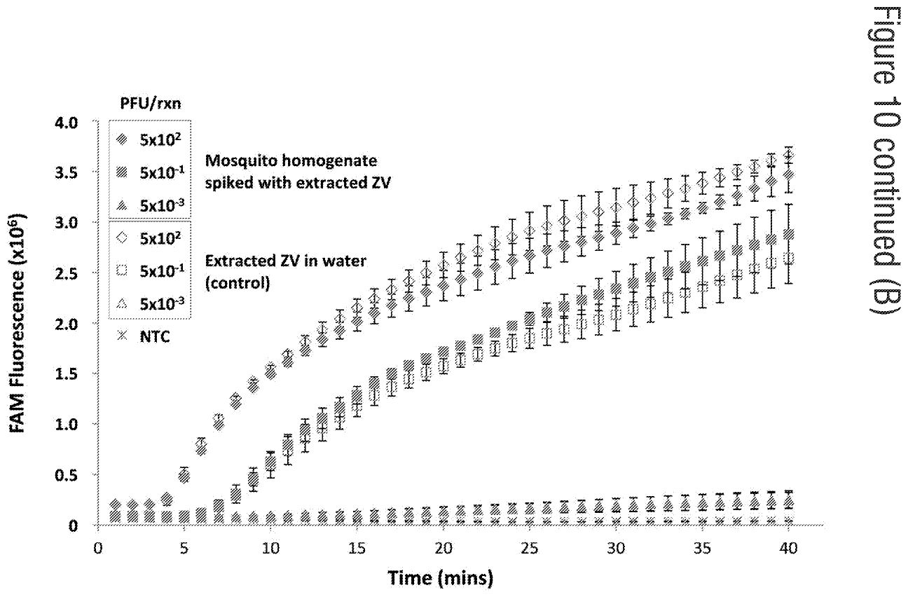

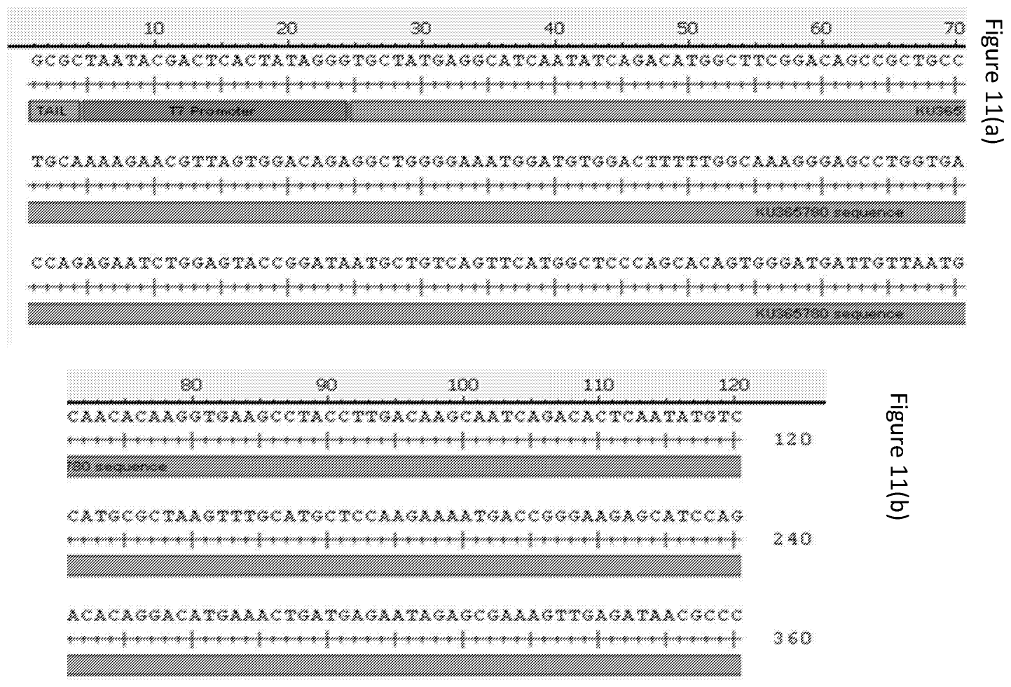

FIG. 9: Effects of crude samples on the sensitivity of the tested RPA assay. Panel a) results obtained in the presence of serum; panel b) results obtained in the presence of urine; panel c) results obtained in the presence of mosquito larvae homogenate; and panel d) results obtained for control (no crude samples)

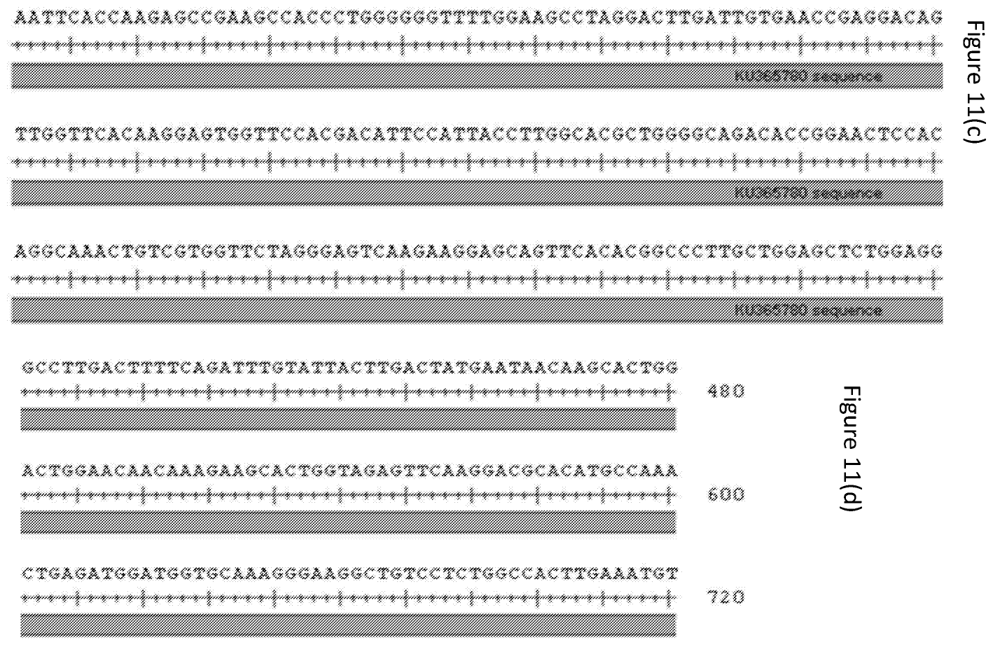

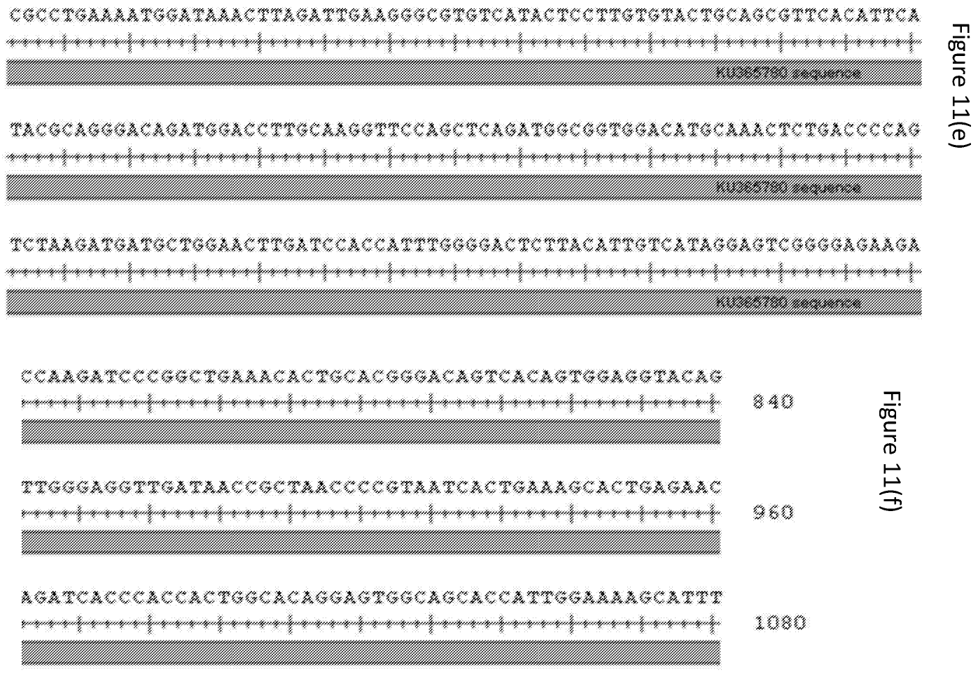

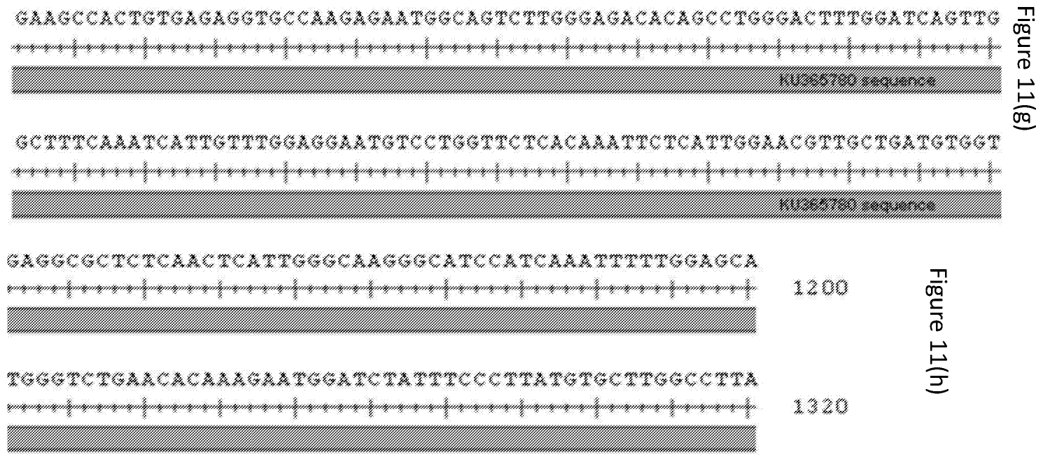

FIG. 10: Effects of crude samples on the sensitivity of the tested RPA assay to detect extracted nucleic acid from cultured Zika virus (South American strain—KU501215). RPA data were generated using exemplary Zika RPA Fw, Zika RPA Rev and Zika RPA Probe 1 of the invention. Panel a) shows results obtained in the presence of semen, urine, saliva and serum samples. Fluorescent signal from these is compared to the signal generated from the amplification of 1×10² PFU per reaction of the same extracted virus in nuclease-free water and the non-template control (NTC). Panel b) shows results obtained in the presence of homogenised mosquito preparations. Amplification of these in the assay of the invention was compared to signal generated for the same dilutions of viral nucleic acid in nuclease-free water and the non-template control (NTC).

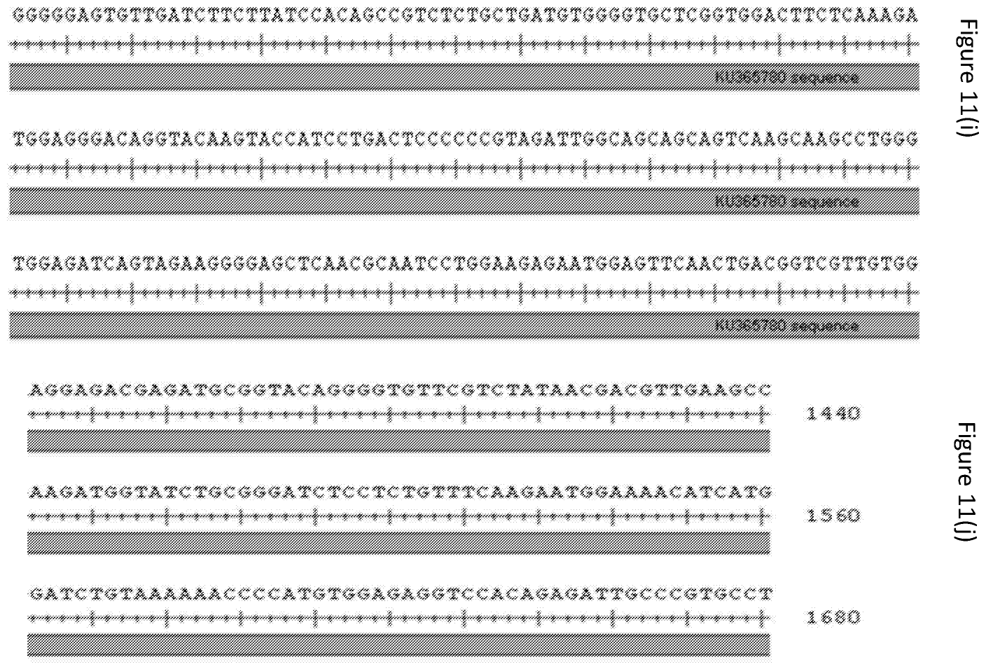

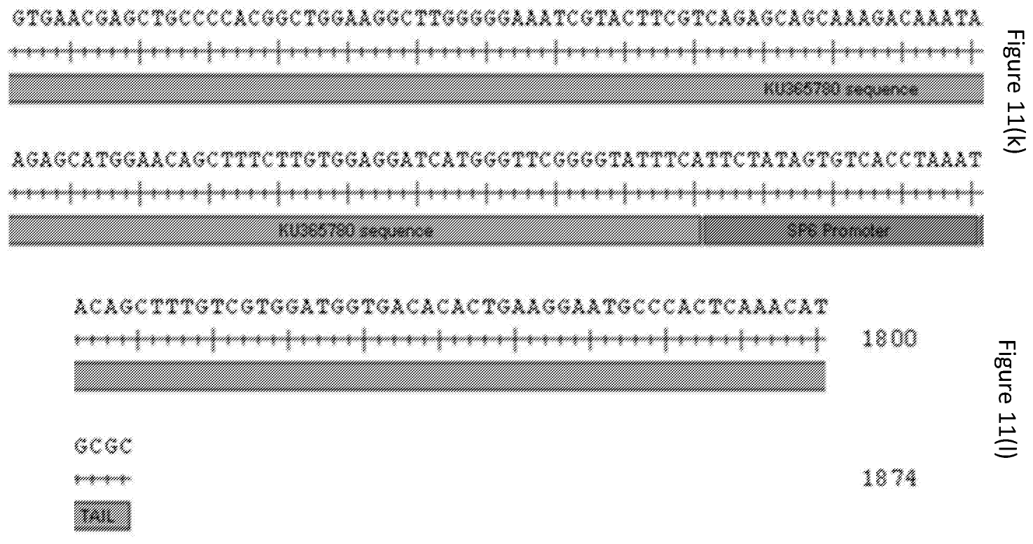

FIG. 11: Synthetic KU365780 Zika DNA fragment sequence.

The inventors have conducted a detailed analysis of the sequences of a selection of South American and Pacific Island Zika outbreak strains. Based on the results of this analysis, the inventors have designed new primers and probes which allow detection of Zika virus sequences from current outbreak strains. Surprisingly, the primers and probes of the invention are also able to detect Zika viruses from distant lineages. This surprising result is highly desirable, because the primers and probes of the invention have the potential to detect all lineages of Zika virus. This reduces the risk of false negatives.

Moreover, the primers and probes of the invention did not show any cross-reactivity with closely-related viral strains, except for Spondweni, which elicited a very weak signal when high levels of template were used. This reduces the risk of false positives.

Another advantage provided by the primers and probes of the invention is that they provide high sensitivity and specificity, even when used with crude sample preparations (both of human and insect origin). The invention therefore avoids the requirement for highly stringent sample preparation, reduces the requirement for skilled technicians, and greatly reduces the cost and time spent on sample preparation.

A further advantage provided by the invention is that the mode of detection of Zika virus is flexible; it can be performed using real-time/quantitative PCR (qPCR), standard PCR, or isothermal techniques such as recombinase polymerase amplification (RPA), depending on the equipment available. The primers and probes of the invention allow this flexibility in the mode of detection because their annealing temperatures are compatible with the reverse transcriptase enzyme. Isothermal methods (such as RPA) are preferred, because they achieve the high sensitivity that is provided by RT-PCR (detecting down to a single genome copy), and are also faster, simpler, more portable, lower cost, and more simple-to-use (and may thus be performed by an untrained operator). These advantages are highly desirable for real-world detection of Zika virus. In a preferred embodiment, the primers and probes of the invention are used in an RPA assay. The RPA assay requires forward and reverse primers (i.e. a “primer pair”) and a probe for detection. The probe is typically a fluorescent probe. Wherein the amplification method is a “conventional” PCR method (such as RT-PCR), the method typically employs primers of the invention (typically a primer pair).

Commercial RPA kits are readily available (e.g. from TwistDX), and contain freeze-dried reagents for long-term storage. The RPA assay is a simple-to-perform, rapid (5-20 minutes run time) isothermal molecular detection method, which shows particularly high sensitivity and speed, even compared to other isothermal methods (e.g. LAMP, SDA, RCA and NASBA). For example, the RPA assay is preferred over loop-mediated-isothermal amplification (LAMP) because it is more amenable to multiplexing and is performed at a lower temperature (37-42 degrees rather than 60-65 degrees), leading to lower power requirements. The RPA assay is preferred over NASBA because it is faster (NASBA is described as requiring 2 hours for amplification). RPA is typically carried out as a one-tube amplification reaction, involving a single low reaction temperature (typically 37-42° C.). Readouts from the RPA assay are flexible and include e.g. gel-based, real-time, simple fluorescence detection and lateral-flow. The Zika RPA assay has the flexibility to be used either in a lightweight portable detection device for field testing, or in a laboratory setting in a rapid, high-throughput system.

Advantageously, the RPA assay accommodates the use of crude sample preparations when used with primers and probes of the invention. In an exemplary RPA assay of the invention, samples are collected in-field and undergo minimal processing (e.g. blood is allowed to clot, serum and/or urine is diluted in a lysis buffer and mosquitoes homogenized in a lysis buffer). A simple workflow involves adding sample to the RPA assay buffer and mixing with freeze-dried pellets. A magnesium start initiates the RPA reaction and the RPA can be followed in real-time at a single low temperature on a portable device. The RPA assay has the flexibility to be used in a rapid, high-throughput test e.g. during an outbreak, and avoids the above-mentioned limitations of conventional PCR and serological detection methods.

The invention is highly-suited to a number of important applications in the detection of Zika virus. For example, the invention can help support local surveillance of Zika virus, in the animal reservoir, in Zika virus-positive mosquito populations, and in humans. This enables rapid targeting of vector control measures including the prediction of emerging Zika virus epizootics before they enter the human population.

The invention also permits faster and more widespread screening of suspect cases than existing methods. The invention is therefore ideally suited to surveillance of Zika virus prevalence (e.g. using human samples). Moreover, the improved detection of Zika virus, provided by the invention, would significantly improve the identification of suitable patients, when therapeutic treatments for Zika virus infection become available.

The invention allows the screening of a variety of samples, such as blood, urine, saliva and organs, and can therefore be used to test for suspected cases, and help avoid transfusion and transplant-mediated transmission of Zika virus. The invention is well-suited to rapid turnaround screening, and is highly applicable to applications where a rapid clinical decision is required e.g. for organ transplant. The invention can also be used in screening travellers returning from a Zika virus affected area (or an area suspected of being affected). The invention can also be used in testing symptomatic individuals and pregnant women, sexual health and family planning screening in affected countries.

The invention is also suitable for diagnosing a Zika virus infection in an individual e.g. for confirming whether an individual suspected of Zika virus infection is infected with Zika virus.

In one embodiment, the individual is an insect (typically a mosquito) or an animal that forms part of the animal reservoir for the virus. An such instances, “infection” refers to a host insect or a reservoir animal that is carrying Zika virus.

In one embodiment, the primers and probes of the invention are used in a multiplex method. In one embodiment, the primers and probes of the invention are used in a multiplex method for detection of Zika virus, as well as other medically important viruses circulating in the outbreak and vectored by mosquitos e.g. Dengue, Chikungunya and Yellow Fever.

The invention provides nucleic acid primers and probes for the detection of Zika virus nucleic acid in a sample.

In one embodiment, detection of Zika virus nucleic acid in a sample comprises the use of a primer pair of the invention (i.e. a forward and reverse primer).

In one embodiment, detection of Zika virus nucleic acid in a sample comprises the use of probe of the invention.

In one embodiment, detection of Zika virus nucleic acid in a sample comprises the use of primers and probes of the invention. A primer pair of the invention and a probe of the invention are typically used in the RPA method.

In one embodiment, the invention provides a composition comprising primers and/or probes of the invention. Said composition may be provided in any form, typically lyophilised, liquid, or frozen. Compositions of the invention typically comprise e.g. salts and/or stabilisers and/or components required for DNA amplification.

Primers and probes of the invention may be referred to as “oligonucleotide” probes and primers.

Primers and probes of the invention are isolated nucleic acids.

“Zika RPA 7 Fw”

A preferred primer of the invention is “Zika RPA 7 Fw” (SEQ ID NO: 1):

CCAACACAAGGTGAAGCCTACCTTGACAAGCAATC

“Zika RPA 7 Fw” is a “forward” primer that binds to the sense strand of the Zika virus “E” (envelope) gene, as shown at FIG. 1(A).

The invention also provides fragments of “Zika RPA 7 Fw” that comprise 15 or more consecutive nucleic acids of SEQ ID NO:1, e.g. at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, or at least 34 consecutive nucleic acids of SEQ ID NO: 1. Preferred fragments of SEQ ID NO: 1 comprise the sequence GTGAAGCCTACCTTG (SEQ ID NO: 2).

The inventors believe that sensitivity of the amplification technique (typically the RPA assay), when using the primers and probe of the invention, may be further improved by slightly modifying the primers and probe described in the Examples, by “frame-shifting” the primers and/or probe by up to 6 bp in the 5′ or 3′ direction or altering the size by +/−15 bp, preferably +/−8 bp (relative to the KU365780 DNA sequence shown in FIG. 1).

Thus, the invention also provides the following forward primers:

“Zika RPA Fw” (SEQ ID NO: 3):

CCAACACAAGGTGAAGCCTACCTTGACAAGCAAT

“Zika RPA Fw” is a preferred “forward” primer that binds to the sense strand of the Zika virus “E” (envelope) gene, as shown at FIG. 1(B).

(SEQ ID NO: 4)
cCCAACACAAGGTGAAGCCTACCTTGACAAGCAAT
(SEQ ID NO: 5)
gcCCAACACAAGGTGAAGCCTACCTTGACAAGCAA
(SEQ ID NO: 6)
tgcCCAACACAAGGTGAAGCCTACCTTGACAAGCA
(SEQ ID NO: 7)
ctgcCCAACACAAGGTGAAGCCTACCTTGACAAGC
(SEQ ID NO: 8)
gctgcCCAACACAAGGTGAAGCCTACCTTGACAAG
(SEQ ID NO: 9)
cgctgcCCAACACAAGGTGAAGCCTACCTTGACAA
(SEQ ID NO: 10)
CAACACAAGGTGAAGCCTACCTTGACAAGCAATCa
(SEQ ID NO: 11)
AACACAAGGTGAAGCCTACCTTGACAAGCAATCag
(SEQ ID NO: 12)
ACACAAGGTGAAGCCTACCTTGACAAGCAATCaga
(SEQ ID NO: 13)
CACAAGGTGAAGCCTACCTTGACAAGCAATCagac
(SEQ ID NO: 14)
ACAAGGTGAAGCCTACCTTGACAAGCAATCagaca
(SEQ ID NO: 15)
CAAGGTGAAGCCTACCTTGACAAGCAATCagacac

The invention also provides fragments of any one of SEQ ID NOs: 1-15 that comprise 15 or more consecutive nucleic acids of any one of SEQ ID NOs: 1-15, e.g. at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, or at least 34 consecutive nucleic acids of any one of SEQ ID NOs: 1-15. Preferred fragments of SEQ ID NOs: 1-15 comprise the sequence GTGAAGCCTACCTTG (SEQ ID NO: 2).

Other preferred fragments lack one or more nucleic acids e.g. 1, 2, 3, 4, 5, 6, 7, 8, 9, or 10 amino acids from the 5′ terminus, and/or one or more nucleic acids e.g. 1, 2, 3, 4, 5, 6, 7, 8, 9, or 10 amino acids from the 3′ terminus of SEQ ID NO: 1 or of SEQ ID NOs: 1-15, while retaining the ability to bind specifically to Zika virus nucleic acid.

Primers of the invention also include variants of any one of SEQ ID NOs: 1-15. Such variants typically consist of a nucleic acid sequence having 85% or more identity, e.g. 85%, 86%, 87%, 88%, 89%, 90%, 91%, 92%, 93%, 94%, 95%, 96%, 97%, 98%, 99% identity to any one of SEQ ID NOs: 1-15.

In some embodiments, the above-disclosed forward primers of the invention comprise additional nucleic acids at the 5′ end of the above-disclosed primer sequences. Preferably, said additional nucleic acids correspond to the nucleic acids in the KU365780 DNA sequence (shown in FIG. 1) that are directly upstream to nucleic acids in the KU365780 DNA sequence that bind to the above-disclosed forward primers. In some embodiments, the above-disclosed primers of the invention comprise up to 15 additional nucleic acids (e.g. 15, 14, 13, 12, 11, 10, 9, 8, 7, 6, 5, 4, 3, 2 or 1 additional nucleic acids) at their 5′ end. Such primers also fall within the scope of the invention.

In some embodiments, the above-disclosed forward primers of the invention comprise additional nucleic acids at the 3′ end of the above-disclosed primer sequences. Preferably, said additional nucleic acids correspond to the nucleic acids in the KU365780 DNA sequence (shown in FIG. 1) that are directly downstream to nucleic acids in the KU365780 DNA sequence that bind to the above-disclosed forward primers. In some embodiments, the above-disclosed primers of the invention comprise up to 15 additional nucleic acids (e.g. 15, 14, 13, 12, 11, 10, 9, 8, 7, 6, 5, 4, 3, 2 or 1 additional nucleic acids) at their 3′ end. Such primers also fall within the scope of the invention.

In some embodiments, the above-disclosed forward primers of the invention comprise additional nucleic acids at the 5′ and 3′ ends of the above-disclosed primer sequences.

“Zika RPA 11 Rev”

A preferred primer of the invention is “Zika RPA 11 Rev” (SEQ ID NO: 16):

ATTCTCTGGCTGGATGCTCTTCCCGGTCATTTTCT

“Zika RPA 11 Rev” is a “reverse” primer that binds to the reverse strand of the Zika virus “E” (envelope) gene, as shown at FIG. 1(A).

The invention also provides fragments of “Zika RPA 11 Rev” that comprise 15 or more consecutive nucleic acids of SEQ ID NO: 16, e.g. at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, or at least 34 consecutive nucleic acids of SEQ ID NO: 16. Preferred fragments of SEQ ID NO: 16 comprise the sequence TGGATGCTCTTCCCG (SEQ ID NO: 17).

The inventors believe that sensitivity of the RPA assay, when using the primers and probe of the invention, may be further improved by slightly modifying the primers and probe described in the Examples, by “frame-shifting” the primers and/or probe by up to 6 bp in the 5′ or 3′ direction or altering the size by +/−15 bp, preferably +/−8 bp (relative to the KU365780 DNA sequence shown in FIG. 1).

Thus, the invention also provides the following reverse primers:

“Zika RPA Rev” (SEQ ID NO: 18):

TTCTCTGGCTGGATGCTCTTCCCGGTCATTTTC

“Zika RPA Rev” is a preferred “reverse” primer that binds to the reverse strand of the Zika virus “E” (envelope) gene, as shown at FIG. 1(B).

(SEQ ID NO: 19)
aATTCTCTGGCTGGATGCTCTTCCCGGTCATTTTC
(SEQ ID NO: 20)
caATTCTCTGGCTGGATGCTCTTCCCGGTCATTTT
(SEQ ID NO: 21)
ccaATTCTCTGGCTGGATGCTCTTCCCGGTCATTT
(SEQ ID NO: 22)
tccaATTCTCTGGCTGGATGCTCTTCCCGGTCATT
(SEQ ID NO: 23)
ctccaATTCTCTGGCTGGATGCTCTTCCCGGTCAT
(SEQ ID NO: 24)
gctccaATTCTCTGGCTGGATGCTCTTCCCGGTCA
(SEQ ID NO: 25)
TTCTCTGGCTGGATGCTCTTCCCGGTCATTTTCTc
(SEQ ID NO: 26)
TCTCTGGCTGGATGCTCTTCCCGGTCATTTTCTct
(SEQ ID NO: 27)
CTCTGGCTGGATGCTCTTCCCGGTCATTTTCTctg
(SEQ ID NO: 28)
TCTGGCTGGATGCTCTTCCCGGTCATTTTCTctgg
(SEQ ID NO: 29)
CTGGCTGGATGCTCTTCCCGGTCATTTTCTctgga
(SEQ ID NO: 30)
TGGCTGGATGCTCTTCCCGGTCATTTTCTctggag

The invention also provides fragments of any one of SEQ ID NOs: 16-30 that comprise 15 or more consecutive nucleic acids of any one of SEQ ID NOs: 16-30, e.g. at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, or at least 34 consecutive nucleic acids of any one of SEQ ID NOs: 16-30. Preferred fragments of SEQ ID NOs: 16-30 comprise the sequence TGGATGCTCTTCCCG (SEQ ID NO: 17).

Other preferred fragments lack one or more nucleic acids e.g. 1, 2, 3, 4, 5, 6, 7, 8, 9, or 10 amino acids from the 5′ terminus, and/or one or more nucleic acids e.g. 1, 2, 3, 4, 5, 6, 7, 8, 9, or 10 amino acids from the 3′ terminus of SEQ ID NO: 16 or of SEQ ID NOs: 16-30, while retaining the ability to bind specifically to Zika virus nucleic acid.

Primers of the invention also include variants of any one of SEQ ID NOs: 16-30. Such variants typically consist of an amino acid sequence having 85% or more identity, e.g. 85%, 86%, 87%, 88%, 89%, 90%, 91%, 92%, 93%, 94%, 95%, 96%, 97%, 98%, 99% identity to any one of SEQ ID NOs: 16-30.

In some embodiments, the above-disclosed reverse primers of the invention comprise additional nucleic acids at the 5′ end of the above-disclosed primer sequences. Preferably, said additional nucleic acids correspond to the nucleic acids in the KU365780 DNA sequence (corresponding to the reverse complement of the sequence shown in FIG. 1) that are directly upstream to nucleic acids in the KU365780 DNA sequence that bind to the above-disclosed reverse primers. In some embodiments, the above-disclosed primers of the invention comprise up to 15 additional nucleic acids (e.g. 15, 14, 13, 12, 11, 10, 9, 8, 7, 6, 5, 4, 3, 2 or 1 additional nucleic acids) at their 5′ end. Such primers also fall within the scope of the invention.

In some embodiments, the above-disclosed reverse primers of the invention comprise additional nucleic acids at the 3′ end of the above-disclosed primer sequences. Preferably, said additional nucleic acids correspond to the nucleic acids in the KU365780 DNA sequence (corresponding to the reverse complement of the sequence shown in FIG. 1) that are directly downstream to nucleic acids in the KU365780 DNA sequence that bind to the above-disclosed reverse primers. In some embodiments, the above-disclosed primers of the invention comprise up to 15 additional nucleic acids (e.g. 15, 14, 13, 12, 11, 10, 9, 8, 7, 6, 5, 4, 3, 2 or 1 additional nucleic acids) at their 3′ end. Such primers also fall within the scope of the invention.

In some embodiments, the above-disclosed reverse primers of the invention comprise additional nucleic acids at the 5′ and 3′ ends of the above-disclosed primer sequences.

“Zika RPA Probe 1”

A preferred probe of the invention is “Zika RPA Probe 1” (SEQ ID NO: 31):

GAACGTTAGTGGACAGAGGCTGGGGAAATGGA567GGACTTTTTGGC
AAA3

Wherein: 3=propanol,
5=dT-fluorescein,
6=D-spacer,
7=dT-BHQ1

“Zika RPA Probe 1” is an “EXO” probe, which has been designed for use in an RPA assay.

Other modifications may be present at groups 3, 5, 6 and 7. According to the invention:
3=modification that functions to block polymerase extension
5=dT-fluorophore
6=an abasic nucleotide analog
7=dT-quencher group (suitable for group “5”)

In one embodiment:
3=selected from (i) C₃-spacer, (ii) a phosphate, (iii) a biotin-TEG, or (iv) an amine
5=selected from (i) dT-fluorescein, (ii) TAMRA, and Cy5
6=a tetrahydrofuran residue (“THF”, sometimes referred to as a “D-spacer”),
7=selected from (i) dT-BHQ1 (preferably wherein group “5” is dT-fluorescein) and (ii) dT-BHQ2 (preferably wherein group “5” is TAMRA or Cy5)

The probe of the invention is typically 46-52 nucleic acids in length.

Whilst the probes of the invention described herein typically include group “5” at the 5′ of group “7”, the skilled person will appreciate that the relative position of groups “5” and “7”, may be swapped. Said probes fall within the scope of the invention.

Preferred fragments of SEQ ID NO: 31 comprise the sequence AGGCTGGGGAAATGGA567G3 (SEQ ID NO: 32).

As shown in FIGS. 1(A&B), the Zika RPA Probe 1 corresponds to the “GAACGTTAGTGGACAGAGGCTGGGGAAATGGATGTGGACTTTTTGGCAAA” region of the KU365780 sequence, except that the underlined “TGT” residues have been substituted with dT-fluorescein, D-spacer, and dTHQ1, respectively. Probes of the invention comprise groups “5”, “6” and “7”. Groups “5” and “7” are preferably separated by 2 to 6 bases. In a preferred embodiment, groups “5” and “7” are separated by group “6” only (i.e. said groups are preferably positioned directly adjacent to each other).

Whilst SEQ ID NO: 31 includes groups “5”, “6” and “7” at the positions corresponding to nucleic acids “TGT” (as shown above), other nucleic acids may instead be substituted for groups “5”, “6” and “7” (i.e. groups “5”, “6” and “7” may be present at different position within probes of the invention). In a preferred embodiment, groups “5”, “6” and “7” are substituted for nucleic acids “TGT”, as shown above.

In a preferred embodiment, primers of the invention that oppose the direction of the probe do not overlap with the probe, to avoid the occurrence of primer-probe dimers. In a preferred embodiment, secondary structures that could cause probes to fold back on themselves are avoided.

In one embodiment, probe of the invention comprises a sequence selected from:

(SEQ ID NO: 33)
567CTGGGGAAATGGATGTG3
(SEQ ID NO: 34)
A567TGGGGAAATGGATGTG3
(SEQ ID NO: 35)
AG567GGGGAAATGGATGTG3
(SEQ ID NO: 36)
AGG567GGGAAATGGATGTG3
(SEQ ID NO: 37)
AGGC567GGAAATGGATGTG3
(SEQ ID NO: 38)
AGGCT567GAAATGGATGTG3
(SEQ ID NO: 39)
AGGCTG567AAATGGATGTG3
(SEQ ID NO: 40)
AGGCTGG567AATGGATGTG3
(SEQ ID NO: 41)
AGGCTGGG567ATGGATGTG3
(SEQ ID NO: 42)
AGGCTGGGG567TGGATGTG3
(SEQ ID NO: 43)
AGGCTGGGGA567GGATGTG3
(SEQ ID NO: 44)
AGGCTGGGGAA567GATGTG3
(SEQ ID NO: 45)
AGGCTGGGGAAA567ATGTG3
(SEQ ID NO: 46)
AGGCTGGGGAAAT567TGTG3
(SEQ ID NO: 47)
AGGCTGGGGAAATG567GTG3
(SEQ ID NO: 48)
AGGCTGGGGAAATGG567TG3
(SEQ ID NO: 49)
AGGCTGGGGAAATGGA567G3
(SEQ ID NO: 50)
AGGCTGGGGAAATGGAT5673

In one embodiment, probe of the invention comprises a sequence selected from

(SEQ ID NO: 63)
GAACG56AG7GGACAGAGGCTG,
preferably
(SEQ ID NO: 64)
GAACG56AG7GGACAGAGGCTGGGGAAATGGATGTGGACTTTTTGGC
AAA.
(SEQ ID NO: 65)
GAACG5T6G7GGACAGAGGCTGG,
preferably
(SEQ ID NO: 66)
GAACG5T6G7GGACAGAGGCTGGGGAAATGGATGTGGACTTTTTGGC
AAA.
(SEQ ID NO: 67)
GAACG5TA67GGACAGAGGCTGGG,
preferably
(SEQ ID NO: 68)
GAACG5TA67GGACAGAGGCTGGGGAAATGGATGTGGACTTTTTGGC
AAA.
(SEQ ID NO: 69)
GAACG76AG5GGACAGAGGCTG,
preferably
(SEQ ID NO: 70)
GAACG76AG5GGACAGAGGCTGGGGAAATGGATGTGGACTTTTTGGC
AAA.
(SEQ ID NO: 71)
GAACG7T6G5GGACAGAGGCTGG,
preferably
(SEQ ID NO: 72)
GAACG7T6G5GGACAGAGGCTGGGGAAATGGATGTGGACTTTTTGGC
AAA.
(SEQ ID NO: 73)
GAACG7TA65GGACAGAGGCTGGG,
preferably
(SEQ ID NO: 74)
GAACG7TA65GGACAGAGGCTGGGGAAATGGATGTGGACTTTTTGGC
AAA.

In one embodiment, probe of the invention comprises a sequence selected from:

(SEQ ID NO: 75)
AGAGGCTGGGGAAA56GA7GTGGACTTTTTG,
preferably
(SEQ ID NO: 76)
GAACGTTAGTGGACAGAGGCTGGGGAAA56GA7GTGGACTTTTTGGC
AAA.
(SEQ ID NO: 77)
GAGGCTGGGGAAA5G6A7GTGGACTTTTTGG,
preferably
(SEQ ID NO: 78)
GAACGTTAGTGGACAGAGGCTGGGGAAA5G6A7GTGGACTTTTTGGC
AAA.
(SEQ ID NO: 79)
AGGCTGGGGAAA5GG67GTGGACTTTTTGGC,
preferably
(SEQ ID NO: 80)
GAACGTTAGTGGACAGAGGCTGGGGAAA5GG67GTGGACTTTTTGGC
AAA.
(SEQ ID NO: 81)
AGAGGCTGGGGAAA76GA5GTGGACTTTTTG,
preferably
(SEQ ID NO: 82)
GAACGTTAGTGGACAGAGGCTGGGGAAA76GA5GTGGACTTTTTGGC
AAA.
(SEQ ID NO: 83)
GAGGCTGGGGAAA7G6A5GTGGACTTTTTGG,
preferably
(SEQ ID NO: 84)
GAACGTTAGTGGACAGAGGCTGGGGAAA7G6A5GTGGACTTTTTGGC
AAA.
(SEQ ID NO: 85)
AGGCTGGGGAAA7GG65GTGGACTTTTTGGC,
preferably
(SEQ ID NO: 86)
GAACGTTAGTGGACAGAGGCTGGGGAAA7GG65GTGGACTTTTTGGC
AAA.

In one embodiment, probe of the invention comprises a sequence selected from:

(SEQ ID NO: 87)
TGGGGAAATGGATG56GAC7TTTTGGCAAA,
preferably
(SEQ ID NO: 88)
GAACGTTAGTGGACAGAGGCTGGGGAAATGGATG56GAC7TTTTGGC
AAA.
(SEQ ID NO: 89)
GGGGAAATGGATG5G6AC7TTTTGGCAAA,
preferably
(SEQ ID NO: 90)
GAACGTTAGTGGACAGAGGCTGGGGAAATGGATG5G6AC7TTTTGGC
AAA.
(SEQ ID NO: 91)
GGGAAATGGATG5GG6C7TTTTGGCAAA,
preferably
(SEQ ID NO: 92)
GAACGTTAGTGGACAGAGGCTGGGGAAATGGATG5GG6C7TTTTGGC
AAA.
(SEQ ID NO: 93)
GGAAATGGATG5GGA67TTTTGGCAAA,
preferably
(SEQ ID NO: 94)
GAACGTTAGTGGACAGAGGCTGGGGAAATGGATG5GGA67TTTTGGC
AAA.
(SEQ ID NO: 95)
TGGGGAAATGGATG76GAC5TTTTGGCAAA,
preferably
(SEQ ID NO: 96)
GAACGTTAGTGGACAGAGGCTGGGGAAATGGATG76GAC5TTTTGGC
AAA.
(SEQ ID NO: 97)
GGGGAAATGGATG7G6AC5TTTTGGCAAA,
preferably
(SEQ ID NO: 98)
GAACGTTAGTGGACAGAGGCTGGGGAAATGGATG7G6AC5TTTTGGC
AAA.
(SEQ ID NO: 99)
GGGAAATGGATG7GG6C5TTTTGGCAAA,
preferably
(SEQ ID NO: 100)
GAACGTTAGTGGACAGAGGCTGGGGAAATGGATG7GG6C5TTTTGGC
AAA.
(SEQ ID NO: 101)
GGAAATGGATG7GGA65TTTTGGCAAA,
preferably
(SEQ ID NO: 102)
GAACGTTAGTGGACAGAGGCTGGGGAAATGGATG7GGA65TTTTGGC
AAA.

Preferably, probes of the invention comprise 30 or more nucleic acids at the 5′ of group “6”. Said 30 or more nucleic acids typically correspond to the corresponding nucleic acids in the KU365780 sequence.

Preferably, probes of the invention comprise 15 or more nucleic acids at the 3′ of group “6”. Said 15 or more nucleic acids typically correspond to the corresponding nucleic acids in the KU365780 sequence.

Preferably, probes of the invention comprise 30 or more nucleic acids at the 5′ of group “6”, and 15 or more nucleic acids at the 3′ of group “6”. Said 30 or more nucleic acids and said 15 or more nucleic acids typically correspond to the corresponding nucleic acids in the KU365780 sequence.

Within the present disclosure, groups “5”, “6” and “7” are not treated as sequence variants when discussing “consecutive nucleic acids”, “sequence identity” and the like. In such cases, groups “5”, “6” and “7” are assessed as though they are the corresponding nucleic acids in the KU365780 sequence.

Independent of the nucleic acid sequence in the probe, group “3” is present at the 3′ end of the probe. Group “3” is present at the 3′ end of the probe to block the polymerase extension. In a preferred embodiment, group “3” is propanol.

The inventors believe that sensitivity of the RPA assay, when using the primers and probe of the invention, may be further improved by slightly modifying the primers and probe described in the Examples, by “frame-shifting” the primers and/or probe by up to 6 bp in the 5′ or 3′ direction or altering the size by +/−15 bp, preferably +/−8 bp (relative to the KU365780 DNA sequence shown in FIG. 1).

Thus, the invention also provides the following probes:

(SEQ ID NO: 51)
aGAACGTTAGTGGACAGAGGCTGGGGAAATGGA567GGACTTTTTGGC
AA3
(SEQ ID NO: 52)
aaGAACGTTAGTGGACAGAGGCTGGGGAAATGGA567GGACTTTTTGG
CA3
(SEQ ID NO: 53)
aaaGAACGTTAGTGGACAGAGGCTGGGGAAATGGA567GGACTTTTTG
GC3
(SEQ ID NO: 54)
aaaaGAACGTTAGTGGACAGAGGCTGGGGAAATGGA567GGACTTTTT
GG3
(SEQ ID NO: 55)
caaaaGAACGTTAGTGGACAGAGGCTGGGGAAATGGA567GGACTTTT
TG3
(SEQ ID NO: 56)
gcaaaaGAACGTTAGTGGACAGAGGCTGGGGAAATGGA567GGACTTT
TT3
(SEQ ID NO: 57)
AACGTTAGTGGACAGAGGCTGGGGAAATGGA567GGACTTTTTGGCAA
Ag3
(SEQ ID NO: 58)
ACGTTAGTGGACAGAGGCTGGGGAAATGGA567GGACTTTTTGGCAAA
gg3
(SEQ ID NO: 59)
CGTTAGTGGACAGAGGCTGGGGAAATGGA567GGACTTTTTGGCAAAg
gg3
(SEQ ID NO: 60)
GTTAGTGGACAGAGGCTGGGGAAATGGA567GGACTTTTTGGCAAAgg
ga3
(SEQ ID NO: 61)
TTAGTGGACAGAGGCTGGGGAAATGGA567GGACTTTTTGGCAAAggg
ag3
(SEQ ID NO: 62)
TAGTGGACAGAGGCTGGGGAAATGGA567GGACTTTTTGGCAAAggga
gc3

As noted above, whilst SEQ ID NO: 51-62 includes groups “5”, “6” and “7” at the positions corresponding to nucleic acids “TGT” in the KU365780 sequence (as shown above), other nucleic acids may instead be substituted for groups “5”, “6” and “7” (i.e. groups “5”, “6” and “7” may be present at different position within probes of the invention). Thus, the invention also provides the following probes:

(SEQ ID NO: 103)
aGAACGTTAGTGGACAGAGGCTGGGGAAATGGATGTGGACTTTTTGGC
AA3
(SEQ ID NO: 104)
aaGAACGTTAGTGGACAGAGGCTGGGGAAATGGATGTGGACTTTTTGG
CA3
(SEQ ID NO: 105)
aaaGAACGTTAGTGGACAGAGGCTGGGGAAATGGATGTGGACTTTTTG
GC3
(SEQ ID NO: 106)
aaaaGAACGTTAGTGGACAGAGGCTGGGGAAATGGATGTGGACTTTTT
GG3
(SEQ ID NO: 107)
caaaaGAACGTTAGTGGACAGAGGCTGGGGAAATGGATGTGGACTTTT
TG3
(SEQ ID NO: 108)
gcaaaaGAACGTTAGTGGACAGAGGCTGGGGAAATGGATGTGGACTTT
TT3
(SEQ ID NO: 109)
AACGTTAGTGGACAGAGGCTGGGGAAATGGATGTGGACTTTTTGGCAA
Ag3
(SEQ ID NO: 110)
ACGTTAGTGGACAGAGGCTGGGGAAATGGATGTGGACTTTTTGGCAAA
gg3
(SEQ ID NO: 111)
CGTTAGTGGACAGAGGCTGGGGAAATGGATGTGGACTTTTTGGCAAAg
gg3
(SEQ ID NO: 112)
GTTAGTGGACAGAGGCTGGGGAAATGGATGTGGACTTTTTGGCAAAgg
ga3
(SEQ ID NO: 113)
TTAGTGGACAGAGGCTGGGGAAATGGATGTGGACTTTTTGGCAAAggg
ag3
(SEQ ID NO: 114)
TAGTGGACAGAGGCTGGGGAAATGGATGTGGACTTTTTGGCAAAggga
gc3;

wherein said probes comprise groups “5”, “6” and “7”, as defined herein.

The invention also provides fragments of any one of SEQ ID NOs: 31-114 that comprise 15 or more consecutive nucleic acids of any one of SEQ ID NOs: 31-114, e.g. at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or 50 consecutive nucleic acids of any one of SEQ ID NOs: 33-44 or 31-114. Preferred fragments of SEQ ID NOs: 31-114 comprise the sequence GAGGCTGGGGAAATGGA567 (SEQ ID NO: 32).

Other preferred fragments lack one or more nucleic acids e.g. 1, 2, 3, 4, 5, 6, 7, 8, 9, or 10 amino acids from the 5′ terminus, and/or one or more nucleic acids e.g. 1, 2, 3, 4, 5, 6, 7, 8, 9, or 10 amino acids from the 3′ terminus of SEQ ID NO: 31 or SEQ ID NOs: 31-114, while retaining the ability to bind specifically to Zika virus nucleic acid.

Probes of the invention also include variants of any one of SEQ ID NOs: 31-114. Such variants typically consist of an amino acid sequence having 85% or more identity, e.g. 85%, 86%, 87%, 88%, 89%, 90%, 91%, 92%, 93%, 94%, 95%, 96%, 97%, 98%, 99% identity to any one of SEQ ID NOs: 31-114. In one embodiment, probes of the invention consist of any one of SEQ ID NOs: 31-114.

In some embodiments, the above-disclosed probes of the invention comprise additional nucleic acids at the 5′ end of the above-disclosed probe sequences. Preferably, said additional nucleic acids correspond to the nucleic acids in the KU365780 DNA sequence (shown in FIG. 1) that are directly upstream to nucleic acids in the KU365780 DNA sequence that bind to the above-disclosed probes. In some embodiments, the above-disclosed probes of the invention comprise up to 15 additional nucleic acids (e.g. 15, 14, 13, 12, 11, 10, 9, 8, 7, 6, 5, 4, 3, 2 or 1 additional nucleic acids) at their 5′ end. Such probes also fall within the scope of the invention.

In some embodiments, the above-disclosed probes of the invention comprise additional nucleic acids at the 3′ end of the above-disclosed probe sequences. Preferably, said additional nucleic acids correspond to the nucleic acids in the KU365780 DNA sequence (shown in FIG. 1) that are directly downstream to nucleic acids in the KU365780 DNA sequence that bind to the above-disclosed probes. In some embodiments, the above-disclosed probes of the invention comprise up to 15 additional nucleic acids (e.g. 15, 14, 13, 12, 11, 10, 9, 8, 7, 6, 5, 4, 3, 2 or 1 additional nucleic acids) at their 3′ end. Such probes also fall within the scope of the invention.

In some embodiments, the above-disclosed probes of the invention comprise additional nucleic acids at the 5′ and 3′ ends of the above-disclosed probe sequences.

All nucleic acid sequences presented herein are presented in a 5′-to-3′ (left-to-right) orientation.

Primers and probes of the invention are capable of binding to the Zika virus “E” (envelope) gene, as shown at FIG. 1. Thus, identification of Zika virus (e.g. in a sample) is based on the detection of nucleic acid sequence(s) that correspond to the Zika virus “E” (envelope) gene. The KU365780 DNA sequence in FIG. 1 provides a Zika virus reference sequence.

Probes of the invention are typically “Exo” probes (typically from TwistDX), for use in an RPA assay.

The invention also relates to other types of probe, for use in combination with primers of the invention. Since the preferred method of the invention involves an RPA assay, said other types of probe are preferably also compatible with the RPA assay, for example “nfo” probes and “fpg” probes.

Thus, in one embodiment, the invention provides primers of the invention and an nfo probe. Nfo probes are typically for use in lateral flow detection, which may involve use of e.g. biotin or digoxigenin tags.

Thus, in one embodiment, the invention provides primers of the invention and a fpg probe, typically from TwistDX. As explained in TwistDX product information sheets, Fpg probes are typically oligonucleotides that are modified at the 5′ end with a quencher group and that contain a fluorophore label on an abasic nucleotide analogue 4 to 5 nucleotides downstream of the quencher (i.e. at position 5 or 6). The fluorophore is attached to the ribose of the abasic site via a C—O—C linker (a so-called dR-group). In addition, TwistAmpR fpg probes are blocked from polymerase extension by a suitable 3′ modification (such as a C3-spacer, a phosphate, a Biotin-TEG or an amine). The fluorescent signal generated by the fluorophore (typically Carboxy-fluorescein) will normally be quenched by the 5′ quencher group (typically a Black Hole Quencher (BHQ)). In a double stranded context the dR-fluorophore residue, the ‘gap’ in the probe, presents a substrate for a number of DNA repair enzymes, including the enzyme fpg present in the TwistAmpR fpg kit.

Typically the % sequence identity is determined over a length of contiguous nucleic acid residues. A primer or probe specific for the Zika virus nucleic acid may, for example, have at least 80% sequence identity to Zika virus nucleic acid, measured over at least 10, at least 11, at least 12, at least 13, at least 14, at least 15, at least 16, at least 14, at least 18, at least 19, at least 20, at least 21, at least 22, at least 23, at least 24, at least 25, at least 26, at least 27, at least 28, at least 29, at least 30, at least 35, at least 40, at least 50, at least 60, at least 70, at least 80 or more nucleic acid residues, up to the entire length of the primer or probe specific for the Zika virus nucleic acid.

Sequence identity may be determined with respect to any primer or probe disclosed herein.

Primers and probes of the invention may be complementary to the Zika virus nucleic acid. Typically the primer or probe specific for Zika virus nucleic acid is complementary over a length of contiguous nucleic acid residues. Typically the primer or probe specific for Zika virus nucleic acid is complementary over a length of at least 10, at least 11, at least 12, at least 13, at least 14, at least 15, at least 16, at least 14, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 70, at least 80, at least 90, or more nucleic acid residues, up to the full length of the primer or probe.

A primer or probe of the invention may be complementary to the reverse sequence of the Zika virus nucleic acid. Typically the primer or probe specific for Zika virus nucleic acid is complementary over a length of contiguous nucleic acid residues of the reverse sequence. Typically, the primer or probe specific for Zika virus nucleic acid is complementary over a length of at least 10, at least 11, at least 12, at least 13, at least 14, at least 15, at least 16, at least 14, at least 18, at least 19, at least 20, at least 21, at least 22, at least 23, at least 24, at least 25, at least 26, at least 27, at least 28, at least 29, at least 30, at least 35, at least 36, at least 37, at least 38, at least 39, at least 40, at least 45, at least 50, at least 55, at least 60, at least 70, at least 80, at least 90, or more nucleic acid residues, up to the full length of the primer or probe.

A primer or probe of the invention may be complementary to a variant of a Zika virus nucleic acid. Typically the primer or probe of the invention is complementary to a variant having at least 80% sequence identity to the Zika virus nucleic acid. A sequence identity of at least 80% includes at least 82%, at least 84%, at least 86%, at least 88%, at least 90%, at least 91%, at least 92%, at least 93%, at least 94%, at least 95%, at least 96%, at least 97%, at least 98%, at least 99%, and 100% sequence identity (to each and every nucleic acid sequence presented herein and/or to each and every SEQ ID NO presented herein).

Variants of the specific sequences provided above may be defined by reciting the number of nucleotides that differ between the variant sequences and the specific reference sequence, preferably with reference to the KU365780 DNA sequence in FIG. 1. These differences may result from the addition, deletion and/or substitution of one or more nucleotide position within the variant sequence compared with the reference sequence. Thus, in one embodiment, the sequence may comprise (or consist of) a nucleotide sequence that differs from the specific sequences provided at no more than ten nucleotide positions, no more than nine nucleotide positions, no more than eight nucleotide positions, no more than seven nucleotide positions, no more than six nucleotide positions, no more than five nucleotide positions, no more than four nucleotide positions, no more than three nucleotide positions, no more than two nucleotide positions or no more than one nucleotide position. Conservative substitutions are preferred. The term variants as defined herein also encompasses splice variants. As noted above, when specifically discussing probes of the invention, groups “5”, “6” and “7” are not treated as sequence variant, and are assessed as though they are the corresponding nucleic acids in the KU365780 sequence.

Typically, for RPA-based methods, the probe is typically 10 to 80 nucleotides in length, preferably 20 to 80 nucleotides in length, more preferably 30 to 70 nucleotides in length, even more preferably 40 to 60 nucleotides in length, and even more preferably 46 to 54 nucleotides in length.

The forward primer of the invention is typically 15-50 nucleotides in length. For RPA-based methods, the forward primer is preferably 15-45 nucleotides in length, more preferably 30-40 nucleotides in length, most preferably 33-37 nucleotides in length.

The reverse primer of the invention is typically 15-50 nucleotides in length. For RPA-based methods, the reverse primer is preferably 15-45 nucleotides in length, more preferably 30-40 nucleotides in length, most preferably 33-37 nucleotides in length.

The primers and probes of the invention are specially designed to hybridise to specific Zika virus nucleic acid. In the context of the present invention, the term “hybridises” includes hybridising to the sense strand of a target sequence, the reverse of a target sequence, the complement of a target sequence or the reverse complement of a target sequence.

Wherein the amplification method is PCR, it is preferred that the binding conditions for primers and probes of the invention are such that a high level of specificity is provided—i.e. hybridisation of the primers and/or probes occurs under “stringent conditions”. In general, stringent conditions are selected to be about 5° C. lower than the thermal melting point (Tm) for the specific sequence at a defined ionic strength and pH. The Tm is the temperature (under defined ionic strength and pH) at which 50% of the target (or complement) sequence hybridises to a perfectly matched probe or primer. In this regard, the Tm of oligonucleotides, probes or primers of the present invention, at a salt concentration of about 0.02M or less at pH 7, is for example above 60° C., such as about 70° C.

Premixed buffer solutions are commercially available (eg. EXPRESSHYB Hybridisation Solution from CLONTECH Laboratories, Inc.), and hybridisation can be performed according to the manufacturer's instructions.

Examples of suitable labels include detectable labels such as radiolabels or fluorescent or coloured molecules, enzymatic markers or chromogenic markers—e.g. dyes that produce a visible colour change upon hybridisation of the probe or primer. By way of example, the label may be digoxygenin, fluorescein-isothiocyanate (FITC), R-phycoerythrin, Alexa 532, carboxy-X-rhodamine (ROX), carboxytetramethylrhodamine (TAMRA), 4,5-dichloro-dimethoxy-fluorescein (JOE), BHQ-1/2/3, Cy5, Cy5.5 or Cy3. The probes or primer preferably contain a Fam label (e.g. a 5′ Fam label), and/or a minor groove binder (MGB). The label may be a reporter molecule, which is detected directly, such as by exposure to photographic or X-ray film. Alternatively, the label is not directly detectable, but may be detected indirectly, for example, in a two-phase system. An example of indirect label detection is binding of an antibody to the label.

Examples of suitable tags include “complement/anti-complement pairs”. The term “complement/anti-complement pair” denotes non-identical moieties that form a non-covalently associated, stable pair under appropriate conditions. Examples of suitable tags include biotin and streptavidin (or avidin). By way of example, a biotin tag may be captured using streptavidin, which may be coated onto a substrate or support such as a bead (for example a magnetic bead) or membrane. Likewise, a streptavidin tag may be captured using biotin, which may be coated onto a substrate or support such as a bead (for example a magnetic bead) or membrane. Other exemplary complement/anti-complement pairs include receptor/ligand pairs, antibody/antigen (or hapten or epitope) pairs, and the like. Another example is a nucleic acid sequence tag that binds to a complementary sequence. The latter may itself be pre-labelled, or may be attached to a surface (e.g. a bead) which is separately labelled. An example of the latter embodiment is the well-known LuminexR bead system. Other exemplary pairs of tags and capture molecules include receptor/ligand pairs and antibody/antigen (or hapten or epitope) pairs. Where subsequent dissociation of the complement/anti-complement pair is desirable, the complement/anti-complement pair has a binding affinity of, for example, less than 10⁹ M⁻¹. One exemplary tagged oligonucleotide, probe or primer is a biotin-labelled oligonucleotide, probe or primer, which may be detected using horse-radish peroxidase conjugated streptavidin.

The probes or primers of the invention may be labelled with different labels or tags, thereby allowing separate identification of each, probe or primer when used in a method of the present invention.

Any conventional method may be employed to attach nucleic acid tags to a probe or primer of the present invention (e.g. to the 5′ end of the defined binding region of the oligonucleotide, probe or primer). Alternatively, oligonucleotides, probes or primers of the invention (with pre-attached nucleic acid tags) may be constructed by commercial providers.

Detection of the Zika virus nucleic acid may be carried out by any known means. In this regard, the probe or amplification product may be tagged and/or labelled, and the detection method may therefore comprise detecting said tag and/or label. Zika virus detection is preferably performed using an isothermal technique, most preferably RPA.

In one embodiment, the probe(s) or primer(s) of the invention comprise a tag and/or label. Thus, in one embodiment, following hybridisation of tagged/labelled probe/primer to target Zika virus nucleic acid, the tag/label becomes associated with the target nucleic acid. Thus, in one embodiment, the assay may comprise detecting the tag/label and correlating presence of tag/label with presence of Zika virus nucleic acid.

In one embodiment, tag and/or label may be incorporated during extension of the probe(s) or primer(s) of the invention. In doing so, the amplification product(s) become tagged/labelled, and the assay may therefore comprise detecting the tag/label and correlating presence of tag/label with presence of amplification product, and hence the presence of Zika virus nucleic acid.

By way of example, in one embodiment, the amplification product may incorporate a tag/label (e.g. via a tagged/labelled dNTP such as biotin-dNTP) as part of the amplification process, and the assay may further comprise the use of a binding partner complementary to said tag (e.g. streptavidin) that includes a detectable tag/label (e.g. a fluorescent label, such as R-phycoerythrin). In this way, the amplified product incorporates a detectable tag/label (e.g. a fluorescent label, such as R-phycoerythrin).

In one embodiment, the probe(s) or primer(s) and/or the amplification product(s) may include a further tag/label (as the complement component) to allow capture of the amplification product(s).

By way of example, a “complement/anti-complement” pairing may be employed in which an anti-complement capture component binds to said further tag/label (complement component) and thereby permits capture of the probe(s) and/or amplification product(s). Examples of suitable “complement/anti-complement” partners have been described earlier in this specification, such as a complementary pair of nucleic acid sequences, a complementary antibody-antigen pair, etc. The anti-complement capture component may be attached (e.g. coated) on to a substrate or solid support—examples of suitable substrates/supports include membranes and/or beads (e.g. a magnetic or fluorescent bead). Capture methods are well known in the art. For example, LuminexR beads may be employed. Alternatively, the use of magnetic beads may be advantageous because the beads (plus captured, tagged/labelled amplification product) can easily be concentrated and separated from the sample, using conventional techniques known in the art.

Immobilisation provides a physical location for the probes, primers and/or anti-complement capture component of the invention, and may serve to fix the capture component/probe/primer at a desired location and/or facilitate recovery or separation of probe/primer. For example, this may be employed as part of a nfo lateral flow assay of the invention, which may employ the use of digoxigenin and biotin labels. The support may be a rigid solid support made from, for example, glass, plastic or silica, such as a bead (for example a fluorescent or magnetic bead). Alternatively, the support may be a membrane, such as nylon or nitrocellulose membrane. 3D matrices are also suitable supports for use with the present invention—e.g. polyacrylamide or PEG gels. Immobilisation to a support/platform may be achieved by a variety of conventional means. By way of example, immobilisation onto a support such as a nylon membrane may be achieved by UV cross-linking. Alternatively, biotin-labelled molecules may be bound to streptavidin-coated substrates (and vice-versa), and molecules prepared with amino linkers may be immobilised on to silanised surfaces. Another means of immobilisation is via a poly-T tail or a poly-C tail, for example at the 3′ or 5′ end. Said immobilisation techniques apply equally to the probe component (and primer/primer pair component, if present) of the present invention.

In one embodiment, the probes and/or primers of the invention comprise a nucleic acid sequence tag/label (e.g. attached to each probe at the 5′ end of the defined sequence of the probe/primer that binds to target/complement nucleic acid). In more detail, each of the probes/primers is provided with a different nucleic acid sequence tag/label, wherein each of said tags/labels (specifically) binds to a complementary nucleic acid sequence present on the surface of a bead. Each of the different tags/labels binds to its complementary sequence counterpart (and not to any of the complementary sequence counterparts of the other tags), which is located on a uniquely identifiable bead. In this regard, the beads are uniquely identifiable, for example by means of fluorescence at a specific wavelength. Thus, in use, probes/primers of the invention bind to Zika virus nucleic acid (if present in the sample). Thereafter, (only) the bound probes may be extended (in the 3′ direction) in the presence of one or more labelled dNTP (e.g. biotin labelled dNTPs, such as biotin-dCTPs).

The extended primers may be contacted with a binding partner counterpart to the labelled dNTPs (e.g. a streptavidin labelled fluorophore, such as streptavidin labelled R-phycoerythrin), which binds to those labelled dNTPs that have become incorporated into the extended primers. Thereafter, the labelled extended primers may be identified by allowing them to bind to their nucleic acid counterparts present on the uniquely identifiable beads. The latter may then be “called” (e.g. to determine the type of bead present by wavelength emission) and the nature of the primer extension (and thus the type of target/complement nucleic acid present) may be determined.

Typically, probes/primers of the invention are oligonucleotides having sequence identity or complementarity with Zika virus nucleic acid (either the sense strand, the complementary strand or the reverse of either strand) as disclosed herein. One or more probe may be immobilised on a solid support, and used to interrogate RNA obtained from a test sample. One or more probe may be immobilised on a solid support, and used to interrogate mRNA or DNA obtained from a test sample. If the RNA from the test sample contains the Zika virus nucleic acid targeted by the immobilised probe, it will bind to the probe, and may then be detected. If the mRNA or DNA from the test sample contains the Zika virus nucleic acid targeted by the immobilised probe, it will bind to the probe, and may then be detected. The probes/primers of the invention may also be detected using PCR, such as real time PCR.

The inventors have developed new primer and probe sequences for the detection of Zika virus. These primers and probes have been carefully designed to allow detection of Zika virus sequences from current outbreak strains, and were surprisingly found to also detect Zika virus from a distant lineage, and so the risk of false negatives is reduced. Advantageously, the primers and probes of the invention did not show any cross-reactivity whatsoever, with closely-related viral strains, and so the risk of false positives is also reduced.

Diagnosing a Zika virus infection in an individual means to identify or detect the presence and/or amount of one Zika virus in the individual. This is achieved by determining the presence and/or amount of Zika virus nucleic acid in a sample, as described herein.

Because of the sensitivity of the present invention to detect a Zika virus infection before an overtly observable clinical manifestation, the diagnosis, identification or detection of a Zika virus infection includes the detection of the onset of a Zika virus infection, as defined above.

According to the present invention, Zika virus infection may be diagnosed or detected, by determining the presence and/or amount Zika virus nucleic acid in a sample obtained from an individual. As used herein, “obtain” means “to come into possession of”. The present invention is particularly useful in predicting and diagnosing a Zika virus infection in an individual, who is suspected of having a Zika virus infection, or who is at risk of a Zika virus infection. The present invention may be used to confirm a clinical suspicion of a Zika virus infection.

The presence and/or amount of Zika virus in a sample may be measured relative to a control or reference population, for example relative to the corresponding Zika virus of a control or reference population. Herein the terms “control” and “reference population” are used interchangeably.

The control or reference population can be generated from one individual or a population of two or more individuals. The control or reference population, for example, may comprise three, four, five, ten, 15, 20, 30, 40, 50 or more individuals. Furthermore, the control or reference population and the individual's (test) sample that are compared in the methods of the present invention may be generated from the same individual, provided that the test and reference samples are taken at different time points and compared to one another. For example, a sample may be obtained from an individual at the start of a study period. A control or reference taken from that sample may then be compared to subsequent samples from the same individual. Such a comparison may be used, for example, to determine the progression of a Zika virus infection in the individual by repeated classifications over time.

The control or reference may be obtained, for example, from a population of Zika virus negative individuals (i.e. individuals negative for infection by Zika virus) or Zika virus positive individuals (i.e. individuals positive for infection by Zika virus).

Typically the control or reference population does not comprise Zika virus and/or is not infected with Zika virus (i.e. is negative for Zika virus infection). Alternatively, the control or reference population may comprise Zika virus and/or be infected with Zika virus (i.e. is positive for Zika virus infection) and may be subsequently diagnosed with a Zika virus infection using conventional techniques. For example, a population of Zika virus infection-positive individuals used to generate the reference or control may be diagnosed with Zika virus infection about 24, 48, 72, 96 or more hours after biological samples were taken from them for the purposes of generating a reference or control. In one embodiment, the population of Zika virus-positive individuals is diagnosed with Zika virus infection using conventional techniques about 0-36 hours, about 36-60 hours, about 60-84 hours, or about 84-108 hours after the biological samples were taken.

As described herein, the present invention relates to a method for determining the presence and/or amount of Zika virus and/or diagnosing a Zika virus infection. Thus, in some instances it is sufficient to detect the presence of Zika virus in a sample. In such cases, the control or reference population may be positive or negative for Zika virus and/or Zika virus infection.

In other instances, the amount of Zika virus is determined relative to a control or reference population. In such cases, the amount of Zika virus is typically increased compared with a control or reference population, the amount may be increased by at least 10%, at least 20%, at least 30%, at least 40%, at least 50%, at least 60, at least 70%, at least 80%, at least 90%, at least 100%, at least 150%, at least 200% compared with the control or reference population.

Alternatively, if the control or reference population is positive for Zika virus, the amount of the Zika virus may be decreased compared with the control or reference population. For example, the amount may be decreased by at least 10%, at least 20%, at least 30%, at least 40%, at least 50%, at least 60, at least 70%, at least 80%, at least 90%, at least 95%, at least 99%, up to total elimination of the Zika virus. Such a Zika virus positive control or reference population may, for example, be used to monitor an individual's response to a treatment directed to the Zika virus (once said treatment has been established), such that if the treatment is successful, the amount of Zika virus will decrease relative to the control or reference population over time.

The presence and/or amount of Zika virus according to the present invention is determined by determining the presence and/or amount of Zika virus nucleic acid in a sample.

Measurements of the Zika virus nucleic acid may include, for example, measurements that indicate the presence, concentration, expression level, or any other value associated with the Zika virus nucleic acid.

The presence and/or amount of said Zika virus nucleic acid may be determined by quantitative and/or semi-quantitative and/or qualitative analysis. Thus, the presence of Zika virus may be determined simply by identifying the presence of Zika virus nucleic acid in a sample (qualitative analysis), with no need to determine the amount of the nucleic acid. Alternatively, the amount of the Zika virus nucleic acid be approximated (semi-quantitative analysis). Alternatively, the amount of the Zika virus nucleic acid may be determined (quantitative analysis). RPA is typically a semi-quantitative method.

The amount of the Zika virus nucleic acid encompasses, but is not limited to, the mass of the nucleic acid, the molar amount of the nucleic acid, the concentration of the nucleic acid, the molarity of the nucleic acid and the copy number of the nucleic acid. This amount may be given in any appropriate units. For example, the concentration of the nucleic acid may be given in pg/ml, ng/ml or μg/ml.

The presence and/or amount of the Zika virus nucleic acid may be measured directly or indirectly. For example, the copy number of the Zika virus nucleic acid may be determined using recombinase polymerase amplification (RPA), PCR, qPCR or qRT-PCR. Wherein the Zika virus nucleic acid is RNA, the expression level may be determined, for example using RPA reverse transcription RPA (RT-RPA). In a preferred embodiment RPA is used. The relative presence and/or amount of the Zika virus nucleic acid relative to a control or reference population may be determined using any appropriate technique. Suitable standard techniques are known in the art.

As used herein, “comparison” includes any means to discern at least one difference in the presence and/or amount of the Zika virus nucleic acid in the individual and the control or reference population. Thus, a comparison may include a visual inspection of chromatographic spectra or numerical data, and a comparison may include arithmetical or statistical comparisons of values assigned to expression of the Zika virus nucleic acid in the individual's sample and the control or reference. Such statistical comparisons include, but are not limited to, applying a decision rule or decision tree. If at least one internal standard is used, the comparison to discern a difference between the individual and the reference or control may also include features of these internal standards, such that the presence and/or amount of the Zika virus nucleic acid in the individual's sample is correlated to the internal standards. The comparison can confirm the presence or absence of Zika virus, and thus to detect or diagnose a Zika virus infection.

The presence and/or amount level of Zika virus nucleic acid (and hence the Zika virus) may be alternatively compared with a control or reference population for at least 12 hours, at least 24 hours, at least 30 hours, at least 48 hours, at least 72 hours, at least 96 hours, at least 120 hours, at least 144 hours, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks or more.

Although the invention does not require a monitoring period to diagnose a Zika virus infection, it will be understood that repeated classifications of the individual, i.e., repeated snapshots, may be taken over time until the individual is no longer at risk. Alternatively, the presence and/or amount Zika virus in a sample obtained from the individual may be compared to presence and/or amount of Zika virus in samples obtained from the same individual at different points in time.

As used herein, an “individual” is an animal. The animal may be a mammal, preferably a human or non-human primate. The animal may be an insect, preferably a mosquito, more preferably an Aedes mosquito. The individual can be normal, suspected of having a Zika virus infection or at risk of a Zika virus infection. It will be understood that the term “individual” embraces one or more animals, particularly insects e.g. pooled insect samples.

As used herein, a “patient” refers to mammal, preferably a human. The patient can be normal, suspected of having a Zika virus infection or at risk of a Zika virus infection.

The present invention enables the rapid detection of Zika virus. By way of example, the method of the invention is typically completed within 30 minutes to 1 hour, 15 minutes to 45 minutes, 10 to 30 minutes, 5 to 25 minutes, or 1 to 20 minutes. The method of the invention is typically completed in less than 45 minutes, less than 30 minutes, less than 20 minutes, or less than 10 minutes.

The presence and/or amount of Zika virus, as determined by determining the presence and/or amount of Zika virus nucleic acid may be detected, quantified or determined by any appropriate means.

The presence and/or amount of the Zika virus nucleic acid may be determined in a sample obtained from an individual. Wherein the individual is a mammal, the sample may be any suitable biological material, for example blood, plasma, saliva, serum, sputum, urine, semen, cerebral spinal fluid, cells, a cellular extract, a tissue sample, a tissue biopsy, a stool sample, a swab from any body site, and/or one or more organs. Wherein the individual is a mammal, the sample is typically from blood, serum, urine, saliva, semen and/or organ(s). The precise biological sample that is taken from the individual may vary, but the sampling preferably is minimally invasive and is easily performed by conventional techniques.

Wherein the individual is an insect, the sample is typically homogenised mosquito(s).

The biological sample may be taken from the individual before, during, and/or after treatment for Zika virus. In one embodiment, the sample is taken after treatment for a Zika virus infection has been initiated.

When samples are taken from an individual, extraction of Zika virus RNA may be performed prior to testing according to the present invention. Accordingly, the methods of the present invention may comprise a nucleic acid extraction step (typically an RNA extraction step). Any suitable technique for nucleic acid extraction (typically RNA extraction) extraction may be used. Suitable techniques are known in the art, for example spin column and precipitation techniques. Equally, an automated robotic system for nucleic acid extraction (typically RNA extraction extraction) may be used.

In some embodiments, the sample is a crude sample. A “crude sample” has undergone minimal or no purification. A crude sample may for example, have undergone centrifugation. Wherein the sample is a blood sample, a crude sample may have been left for sufficient time for the blood to clot. Wherein the sample is serum, urine or homogenised mosquitoes the crude sample may be in a lysis buffer. Typically, a crude sample is not an isolated nucleic acid preparation, such as would typically be prepared for use in a conventional RT-PCR assay. For example, a crude sample has typically not undergone acid phenol/chloroform extraction, glass filter, or oligo (dT) chromatography.

In some embodiments, the sample (e.g. crude sample) is not diluted prior to performing the method of the invention. In some embodiments, the sample has been diluted, preferably with water or with a lysis buffer. For example, a sample may be diluted 1 in 10, 1 in 100, 1 in 1000, 1 in 10⁴, 1 in 10⁵, 1 in 10⁶, 1 in 10⁷, or more.

In some embodiments, the sample comprises RNase inhibitor. Suitable RNAse inhibitors are known in the art.

As described herein, the presence or absence of Zika virus in the sample is detected at the nucleic acid level (either quantitatively and/or semi-quantitatively and/or qualitatively). Typically, the detection is semi-quantitative. Thus, the Zika virus nucleic acid may be detected as DNA and/or RNA and may be detected using any appropriate technique. The Zika virus nucleic acid is typically detected as RNA.

Typically the determination of the presence and/or amount of Zika virus nucleic acid is carried out by amplifying said Zika virus nucleic acid, or a target region of said Zika virus nucleic acid or a fragment of said nucleic acid or target region. Amplification may be carried out using methods and platforms known in the art, for example recombinase polymerase amplification (RPA), PCR (for example, with the use of “Fast DNA Polymerase”, Life Technologies), such as real-time or quantitative PCR (qPCR), block-based PCR, ligase chain reaction, glass capillaries, isothermal amplification methods including loop-mediated isothermal amplification, rolling circle amplification transcription mediated amplification, nucleic acid sequence-based amplification, signal mediated amplification of RNA technology, strand displacement amplification, isothermal multiple displacement amplification, helicase-dependent amplification, single primer isothermal amplification, and circular helicase-dependent amplification. If employed, amplification may be carried using any amplification platform. In some embodiments, PCR, preferably q-PCR is used. In a preferred embodiment, an isothermal amplification technique is used. In a particularly preferred embodiment, RPA is used.

Primers of the invention are typically employed to amplify approximately 100-400, for example 100-300, 100-200 or 140-210 base pair regions of the Zika virus nucleic acid.

In RPA, in the presence of a suitable recombinase, single-stranded DNA binding proteins, strand-displacing polymerase and DNA precursors (dATP, dCTP, dGTP and dTTP), forward and reverse primers are extended in a 5′ to 3′ direction, thereby initiating the synthesis of new nucleic acid strands that are complementary to the individual strands of the target nucleic acid. The primers thereby drive amplification of the Zika virus target nucleic acid, thereby generating amplification products comprising said target nucleic acid sequence. The RPA technique is isothermal, meaning that a thermal cycler is not required, making the technique particularly suitable for use as a low-cost point-of-care (POC) test.

In PCR, in the presence of a suitable polymerase and DNA precursors (dATP, dCTP, dGTP and dTTP), forward and reverse primers are extended in a 5′ to 3′ direction, thereby initiating the synthesis of new nucleic acid strands that are complementary to the individual strands of the target nucleic acid. The primers thereby drive amplification of Zika virus target nucleic acid, thereby generating amplification products comprising said target nucleic acid sequence.

Primers of the invention are extended from their 3′ ends (i.e. in a 5′-to-′3′) direction.

In a preferred embodiment, the presence and/or amount of the Zika virus nucleic acid is determined using RPA. Nucleic acid is isolated from a sample and primers of the invention are used to amplify the Zika virus nucleic acid (if present in the sample).

References herein to determining the presence and/or amount of Zika virus nucleic acid in a sample apply equally to determining the presence and/or amount of a region of the nucleic acid, and/or the presence and/or amount one or more fragment of said nucleic acid or target region thereof.

For example, primer pairs as disclosed herein may be used to amplify Zika virus nucleic acid. The amplification products of (RPA or PCR) reaction may then be visualised by any appropriate means. For example, the amplification products may be separated and visualised by agarose gel electrophoresis. As the molecular weight of the Zika virus nucleic acid will be known, the presence of the Zika virus nucleic acid may be readily determined by the band size of the amplification products as run on an agarose gel. This method has the advantage of requiring standard equipment that will be present in most laboratories.

Primer pairs of the invention may be used with RPA or other techniques such as qPCR to determine the presence and/or amount of the Zika virus nucleic acid. Non-specific fluorescent dyes may be used in RPA or qPCR according to the present invention. Standard RPA and qPCR methods using such non-specific fluorescent dyes are known in the art. Preferably, a DNA probe specific for the Zika virus nucleic acid in the sample, said probes further comprising a reporter, may be used. Said reporter may be a fluorescent reporter. Examples of fluorescent reporters (also referred to as fluorescent tags) are also described herein. Instruments enabling fast on-screen detection of genes by qPCR are commercially available (for example, Taqman®).

Typically, RNA is extracted and/or isolated from a sample prior to analysis by a method of the present invention. Any appropriate method may be used to extract and/or isolate and/or purify the RNA. Standard techniques are known in the art and commercial kits are available.

In some embodiments, DNA is extracted and/or isolated from a sample prior to analysis by a method of the present invention. Any appropriate method may be used to extract and/or isolate and/or purify the DNA. Standard techniques are known in the art and commercial kits are available.

Zika virus nucleic acid from a sample (either purified or unpurified) may be labelled via any method (typically amplification) and used to interrogate one or more probe immobilised on a surface. The probe may be any length as defined herein.

Primers and probes of the invention bind specifically to Zika virus nucleic acid. By “specific”, it will be understood that the primers and probes bind to the Zika virus nucleic acid, with no significant cross-reactivity to any other molecule, particularly any other nucleic acid. Cross-reactivity of a nucleic acid or probe of the invention for Zika virus nucleic acid may be considered significant if the nucleic acid or probe binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to a nucleic acid from a related virus, that is not Zika virus (e.g. Dengue virus). Preferably, the primers or probes of the invention bind to the other nucleic acid at less than 20%, less than 15%, less than 10% or less than 5%, less than 2% or less than 1% the strength that it binds to Zika virus nucleic acid.

Primers of the invention do not consist of the following nucleic acid sequences:

(SEQ ID NO: 115)
TTGGTCATGATACTGCTGATTGC
(SEQ ID NO: 116)
CCTTCCACAAAGTCCCTATTGC
(SEQ ID NO: 117)
CCGCTGCCCAACACAAG
(SEQ ID NO: 118)
CCACTAACGTTCTTTTGCAGACAT

Probes of the invention do not consist of any of the following nucleic acid sequences:

(SEQ ID NO: 119)
FAM-CGGCATACAGCATCAGGTGCATAGGAG
(SEQ ID NO: 120)
FAM-AGCCTACCTTGACAAGCAGTCAGACACTCAA

As described herein, the present invention provides a method for screening for the presence of Zika virus in a sample, comprising determining the presence and/or amount of Zika virus nucleic acid in a sample.

As described herein, the present invention provides a method for diagnosing a Zika virus infection in an individual, comprising determining the presence and/or amount of Zika virus nucleic acid in a sample obtained from the individual.

The method may comprise determining the presence and/or amount of Zika virus nucleic acid in a first sample taken from the individual at a single initial point in time and multiple time points thereafter to monitor the efficacy of any treatment and disease resolution, and comparing the presence and/or amount of said Zika virus nucleic acid in said first sample to the presence and/or amount of said Zika virus nucleic acid in a reference or control sample. Said comparison may determine the status of Zika virus infection in the individual with an accuracy, sensitivity and/or specificity of at least about 99%, at least about 98%, at least about 97%, at least about 96%, at least about 95%, at least about 90%, at least about 80%, at least about 70% or at least about 60%. Typically the accuracy, sensitivity and/or specificity is of at least about 80% or at least about 90%.

The method may comprise determining the presence and/or amount of Zika virus nucleic acid in a first sample from the individual; and comparing the presence or amount of the Zika virus nucleic acid in the individual's first sample to the presence and/or amount of the Zika virus nucleic acid in a sample from a reference or control population, said comparison being capable of classifying the individual as belonging to or not belonging to the reference or control population, wherein the comparison determines the status of Zika virus infection in the individual.

The method may further comprise determining the presence and/or amount of Zika virus nucleic acid in a second sample taken from the individual; and comparing the presence and/or amount of the Zika virus nucleic acid in the individual's second sample to the presence and/or amount of the Zika virus nucleic acid in the control or reference sample, wherein the second comparison is capable of classifying the individual as belonging to or not belonging to the control or reference population, and wherein the second comparison determines the status of Zika virus infection in the individual.

The methods of the invention may be repeated at least once, at least twice, at least three times, at least four times, at least five times, or more. The presence and/or amount of the Zika virus nucleic acid can be determined in a separate sample taken from the individual each time the method is repeated.

The methods of the invention may be used to diagnose, detect and/or predict Zika virus infection. The methods of the invention may be used to distinguish between a Zika virus infection and the absence of such an infection. The methods of the invention may be used to identify an individual with a Zika virus infection and/or an uninfected individual. The methods of the invention may also be used to determine the status of a Zika virus infection. Determining the status of a Zika virus infection in an individual may comprise determining the progression or resolution of a Zika virus infection. Determining the status of a Zika virus infection in an individual may comprise determining the presence of a Zika virus infection in an individual.

A Zika virus infection may be diagnosed or predicted prior to the onset of clinical symptoms, and/or as subsequent confirmation after the onset of clinical symptoms. Clinical symptoms include fever, rash, joint pain and/or conjunctivitis. Other symptoms include muscle pain and headache. Accordingly, the present invention allows for more effective therapeutic intervention and/or diagnosis in the pre-symptomatic stage of infection.

The invention also provides the use of one or more primer and/or probe as defined herein in the manufacture of a diagnostic for a Zika virus infection.

The invention also provides kits and devices that are useful in determining the presence and/or amount of Zika virus in a sample.

In some embodiments (e.g. when employing TwistAmp Basic, TwistAmp Basic-RT, TwistAmp exo, TwistAmp exo-RT, TwistAmp fpg, and/or TwistAmp nfo), methods of invention can involve the addition of magnesium acetate to initiate the amplification reaction. Thus, in one embodiment, kits and devices of the invention comprise magnesium acetate. Magnesium acetate is typically provided at a concentration of 200-360 mM, preferably 250-210 mM, most preferably 270-290 mM. In one embodiment, magnesium acetate is provided at 280 mM.

The kits and devices of the present invention comprise at least one primer and/or probe of the invention. The primers and/or probes of the kit or device can be used to determine the presence and/or amount of Zika virus nucleic acid in a sample, according to the present invention.

The primers and/or probes of the invention may be part of an array. The primers and/or probes of the invention may be packaged separately and/or individually. The primers and/or probes of the invention may be immobilised on an inert support.

The kit or device may also comprise at least one internal standard to be used in generating profiles of the one or more Zika virus nucleic acid according to the present invention. Likewise, the internal standards can be any of the classes of compounds described above.

The kits and devices of the present invention also may contain reagents that can be used to detectably label the Zika virus nucleic acid contained in the samples from which the profiles of the Zika virus nucleic acid are generated. For this purpose, the kit or device may comprise antibodies which bind to the probes and/or primers of the invention. The antibodies themselves may be detectably labelled. The kit or device also may comprise a specific binding component, such as an aptamer.

In a preferred embodiment, a kit or device of the invention comprises forward and reverse primers of the invention. In a preferred embodiment, a kit or device of the invention comprises a probe of the invention. Most preferably, a kit or device of the invention comprises forward and reverse primers of the invention and a probe of the invention.

The kits and devices of the present invention may also include other classes of compounds including, but not limited to, proteins (including antibodies), and fragments thereof, peptides, polypeptides, proteoglycans, glycoproteins, lipoproteins, carbohydrates, lipids, additional nucleic acids, organic and inorganic chemicals, and natural and synthetic polymers. The kits and devices of the present invention may also include pharmaceutical excipients, diluents and/or adjuvants. Examples of pharmaceutical adjuvants include, but are not limited to, preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It may also be desirable to include isotonic.

### EXAMPLES

Design of Primers and Probes

To assist in designing primers and probes for the specific detection of Zika virus, the inventors gathered sequence information on a selection of South American/Pacific island Zika virus strains (summarised in FIG. 1). Due to the urgent and unmet need for a rapid, simple and fieldable technology that would enable reliable identification of Zika virus in the current outbreak, the inventors designed their primers and probes based on currently circulating Zika virus strains.

The specific primers and probes were specifically designed to be compatible with isothermal methods, particularly the RPA assay.

Having identified suitable Zika virus strains, the inventors manually identified genomic regions that are stable across these strains. These sites were then assessed for their suitability to bind a (cleavable) Exo probe, and then assessed to determine whether they would yield undesirable secondary structures. The bespoke set of primers and probes are detailed in Table 1 (see “Zika RPA 7 Fw”, “Zika RPA 11 Rev”, and “Zika RPA Probe 1”).

Improved Time to Detection

Existing RT-PCR based methods for the detection of Zika virus suffer from slow detection times. The inventors sought to provide a method that is faster, and so better suited to high-throughput screening and field-use.

The inventors performed a direct comparison between a representative gold-standard E-gene PCR method Adapted from the method published by Lanciotti et al (2008), Genetic and Serologic Properties of Zika Virus Associated with an Epidemic, Yap State, Micronesia, 2007. Emerg Infect Dis. 2008 August; 14(8): 1232-1239; with an isothermal method (RPA) for the detection of Zika virus nucleic acid. Primers and probes used in the E-gene PCR method and the RPA method are provided in Table 1.

TABLE 1

| Name | Description | Sequence (5′-3′) | Strain and position | Modifications |
|---|---|---|---|---|
| Zika RPA 7 Fw | RPA forward primer | CCAACACAAGGTGAAGCC | Zika KU365780 | None |
| | | TACCTTGACAAGCAATC | Position 1200-1234 | |
| Zika RPA 11 Rev | RPA reverse primer | ATTCTCTGGCTGGATGCT | Zika KU365780 | None |
| | | CTTCCCGGTCATTTTCT | Position 1345-1379 | |
| Zika RPA Probe 1 | RPA Exo probe | GAACGTTAGTGGACAGAG | Zika KU365780 | 3 = propanol, |
| | | GCTGGGGAAATGGA67GG | Position 1258-1307 | 5 = FLOURESCEIN |
| | | ACTTTTTGGCAAA | | dT, |
| | | | | 6 = D-SPACER, |
| | | | | 7 = BHQ1 dT |
| Zika RPA Fw | RPA forward primer | CCAACACAAGGTGAAGCC | Zika KU365780 | None |
| | | TACCTTGACAAGCAAT | Position 1200-1233 | |
| Zika RPA Rev | RPA reverse primer | TTCTCTGGCTGGATGCTC | Zika KU365780 | None |
| | | TTCCCGGTCATTTTC | Position 1346-1378 | |
| KU365780 DNA | DNA fragment with | See appendix 1. | Zika KU365780 | T7 and SP6. |
| fragment. | T7 and SP6 | | Position 1155-2980 | promoter sites |
| | promoter sites. | | | |
| ZIKV 1086 | E-gene RT-PCR | CCGCTGCCCAACACAAG | Zika AY632535 | None |
| | forward primer. | | Position 1086-1102 | |
| | (Lanciotti 2008) | | | |
| ZIKV 1162c | E-gene RT-PCR | CCACTAACGTTCTTTTGC | Zika AY632535 | |
| | reverse primer. | AGACAT | Position 1162-1139 | |
| | (Lanciotti 2008) | | | |
| ZIKV 1107-FAM | E-gene RT-PCR | AGCCTACCTTGACAAGCA | Zika AY632535 | Not shown. |
| | probe. | GTCAGACACTCAA | Position 1107-1137 | |
| | (Lanciotti 2008) | | | |

As shown in Table 2 (raw output data provided in FIG. 2), the RPA assay, employing the primers and probes of the invention, significantly outperforms the gold-standard RT-PCR assay, with a Ct of just over 6 for the RPA, (equivalent to detection at 6 minutes following assay onset) for 5×10⁴ copies/reaction of target. This compares to Ct 22.1, for the gold-standard RT-PCR, which is equivalent to detection at 31.7 mins after assay onset.

TABLE 2

| | |
|---|---|
| | RPA employing |
| E-gene PCR | primers and probe |
| (RT-PCR) | of the invention |

| Ct | Time to positive | Ct | Time to positive |
|---|---|---|---|
| (Mins) | (Mins) | (Mins) | (Mins) |
| 22.1 | 31.7 | 6.25 | 6.25 |

RPA data generated using exemplary Zika RPA 7 Fw and Zika RPA 11 Rev primers & Zika RPA Probe 1 of the invention.

The RPA assay, employing the primers and probes of the invention, provides a significant reduction in detection time, and is ideally suited to high-throughput screening and field-use.

Assay Sensitivity

High sensitivity is extremely desirable for the detection of Zika virus. As shown in Table 3, the new rapid RPA assay, employing primers and probes of the invention, is (less than) 100 copies/μl, and a low (sub-threshold) detection is observed at 10 copies/μl (raw output data provided in FIG. 3. The detection sensitivity of the tested primers and probes is highly desirable.

TABLE 3

| Copies/μl template | Ct |
|---|---|
| (5 μl added/reaction) | (2 replicates) |
| NTC | 0, 0 |
| 10 | 0, 0 |
| 100 | 17.6, 19.3 |
| 1000 | 9.0, 10.3 |
| 10,000 | 6.2, 6.3 |

RPA data generated using exemplary Zika RPA 7 Fw and Zika RPA 11 Rev primers & Zika RPA Probe 1 of the invention.

Moreover, the inventors believe that the signal strength of the assay may be further improved by further optimising the assay conditions. Specifically, the inventors believe that sensitivity may be further improved by slightly modifying the tested primer and probe, by “frame-shifting” the primers and/or probe by up to 6 bp in the 5′ or 3′ direction or alter the size by +/−15 bp, preferably +/−8 bp (relative to the KU365780 DNA sequence shown in FIG. 1). Such primers and probes fall within the scope of the present invention.

Indeed, the inventors were able to yet further increase the sensitivity of the assay, by slightly modifying the Zika RPA 7 Fw and Zika RPA 11 Rev primers. Forward primer “Zika RPA Fw” corresponds to Zika RPA 7 Fw, except that the 3′ “C” residue in Zika RPA 7 Fw has been omitted. Reverse primer “Zika RPA Rev” corresponds to Zika RPA 11 Rev, except that the 5′ “A” residue and the 3′ “T” residue in Zika RPA 11 Rev have been omitted. Table 4 shows time to positive (TTP) signal data obtained using Zika RPA Fw, Zika RPA Rev and Zika RPA Probe 1 primers and probes of the invention, to detect different number of Zika synthetic RNA template KU365780 copies per reaction. Zika RT-PCR data are also provided for comparison. Table 4 confirms that the primers and probe of the invention significantly outperform RT-PCR, both in terms of sensitivity and speed.

TABLE 4

| Template 5 | | | | |
|---|---|---|---|---|
| (KU365780) | RT-RPA TTP | RT-RPA | RT-PCR TTP | RT-PCR |
| copies/reaction | (mins) | result | (mins) | result |
| 5 × 10⁶ | 3.38 | + (5/5) | 40.12 | + (3/3) |
| 5 × 10⁵ | 3.84 | + (5/5) | 45.07 | + (3/3) |
| 5 × 10⁴ | 4.76 | + (5/5) | 49.18 | + (3/3) |
| 5 × 10³ | 6.46 | + (5/5) | 53.38 | + (3/3) |
| 5 × 10² | 10.72 | + (5/5) | 57.25 | + (3/3) |
| 5 × 10¹ | 21.73 | + (3/5) | 60.33 | + (3/3) |
| 5 × 10⁰ | 22.20 | + (4/5) | Not detected | − (3/3) |
| 5 × 10⁻¹ | Not detected | − (5/5) | Not detected | − (3/3) |
| NTC | Not detected | − (5/5) | Not detected | − (3/3) |

RPA data generated using exemplary Zika RPA Fw, Zika RPA Rev and Zika RPA Probe 1 of the invention.

Detection of Zika Virus from Different Lineages

The primers and probes of the invention were designed based on currently circulating Zika virus strains. However, the ability to detect other lineages of Zika virus would be highly desirable, as it would reduce the risk of false negatives.

The inventors therefore tested whether the primers and probe of the invention are capable of detecting the current South American outbreak strains, as well as an African lineage pre-outbreak strain (the current outbreak in South America developed from the Asian lineage of Zika, rather than the African lineage).

As shown in Table 5 (raw output data provided in FIG. 4) and Table 6 (raw output data provided in FIG. 5), the RPA assay employing primers and probes of the invention, detected both a representative of the current South American outbreak strains and an African lineage pre-outbreak strain, at all dilutions tested. These data suggest that the invention is able to detect a divergence of Zika sequences. Surprisingly, even though the tested primers and probe were designed based on current outbreak sequences, the invention has the potential to detect all lineages of Zika.

TABLE 5

| | South American | Pre-outbreak | |
|---|---|---|---|
| | outbreak strain | African strain | |
| | (KU501215) | (KY288905) | |
| Virus | Ct | Ct | NTC |
| dilution | (2 replicates) | (2 replicates) | Ct |
| 1 in 10 | 4.1, 4.1 | 10.9, 11.2 | 0 |
| 1 in 100 | 5.0, 5.1 | 8.9, 9.5 | 0 |
| 1 in 1000 | 5.2, 6.1 | 12.0, 12.2 | |
| 1 in 10,000 | 7.3, 7.4 | 16.9, 18.0 | |

RPA data generated using exemplary Zika RPA 7 Fw and Zika RPA 11 Rev primers & Zika RPA Probe 1 of the invention.

TABLE 6

| | | RT-RPA TTP | RT-RPA | RT-PCR TTP | RT-PCR |
|---|---|---|---|---|---|
| Sequence ID | Description | (mins) | result | (mins) | result |
| Template 1 | Synthetic RNA | 8.24 | + (3/3) | 53.75 | + |
| (EU545988) | | | | | |
| Template 2 | Synthetic RNA | 7.72 | + (3/3) | 54.05 | + |
| (KJ776791) | | | | | |
| Template 3 | Synthetic RNA | 7.89 | + (3/3) | 54.24 | + |
| (KU321639) | | | | | |
| Template 4 | Synthetic RNA | 8.13 | + (3/3) | 54.65 | + |
| (KU365778) | | | | | |
| Template 5 | Synthetic RNA | 7.32 | + (3/3) | 53.39 | + |
| (KU365780) | | | | | |
| African Zika virus | Extracted virus | 7.53 | + (3/3) | 49.16 | + |
| (KY288905) | | | | | |
| South American Zika | Extracted virus | 4.52 | + (3/3) | 45.95 | + |
| virus (KU501215) | | | | | |

RPA data generated using exemplary Zika RPA Fw, Zika RPA Rev and Zika RPA Probe 1 of the invention.

Performance Using Basic RPA Kit

The inventors tested whether the primers and probe of the invention are compatible with a basic kit (assay performed using a synthetic nucleic acid template (South American outbreak strain)).

As shown in FIG. 6, a single, strong dominant band is apparent around the expected size of 180 bp, suggesting only the expected region is amplified. This indicates that the primers are highly specific for the target region.

Negative Panel Testing

Antibody-based detection assays for Zika virus suffer from serious cross-reactivity with related viral strains (such as the endemic Dengue virus, West Nile virus etc.), or previous immunisation (e.g. yellow fever vaccination), which creates major challenges in positively identifying Zika virus, and discriminating between viral strains.

To test whether the primers and probes of the invention are capable of specifically detecting Zika virus, the inventors performed an RPA assay using extracted nucleic acid from a number of viruses including those that are related to Zika virus. (The negative viral strain collection was obtained in part from the Rare and Imported Pathogens Laboratory (RIPL), Public Health England; consisting of positive control extracts used in routine diagnostic PCR assays. Other strains were either purchased as extracted RNA from the National Collection of Pathogentic Viruses, Public Health England, or were donated by the Virology and Pathogenesis group, Public Health England).

As shown in Table 6 (raw output data provided in FIG. 7) and in Table 7, the negative panel testing demonstrates that the RPA assay, performed using primers and probe of the invention, is highly specific for Zika, with all of the virus extracts from the control panel failing to produce a signal. Thus, primers and probes of the invention do not show cross-reactivity with other related virus strains endemic to the affected regions, viruses that are closely related to Zika virus, or to viruses that are more divergent. In fact, the only tested non-Zika virus strain that could be detected by primers and probe of the invention was Spondweni, and even then only at a weak level, and using very high template RNA concentrations. Spondweni is not even found circulating in South America.

TABLE 6

| | | Viral | | Assay | |
|---|---|---|---|---|---|
| Viral strains | Ct | strains | Ct | controls | Ct |
| 1 Marburg | 0 | 9 Dobrava | 0 | CDC Zika | 3.5, 3.6, |
| | | | | virus 1 | 4.0 |
| 2 CCHF | 0 | 10 SNV | 0 | NTC | 0 |
| 3 Chikungunya | 0 | 11 Hedra | 0 | | |
| 4 Lassa | 0 | 12 Puumala | 0 | | |
| 5JEV | 0 | 13 Dengue | 0 | | |
| 6 Seoul | 0 | 14 Yellow | 0 | | |
| | | fever | | | |
| 7 TBE | 0 | 15 Hazara | 0 | | |
| 8 Nipah | 0 | 16 Issyk-kul | 0 | | |

RPA data generated using exemplary Zika RPA 7 Fw and Zika RPA 11 Rev primers & Zika RPA Probe 1 of the invention.

TABLE 7

| Family | Virus name | Strain | RT-RPA result |
|---|---|---|---|
| Flaviviridae | Dengue 1 | Hawaii A | Not detected |
| | Dengue 2 | R062 | Not detected |
| | Dengue 3 | TC3 | Not detected |
| | Dengue 4 | TC25 | Not detected |
| | West Nile | NY99 | Not detected |
| | Yellow Fever | FNT | Not detected |
| | St Louis | MSI-7 | Not detected |
| | Encephalitis | | |
| | Powassan | — | Not detected |
| | Usutu | — | Not detected |
| | Karshi | 30517 | Not detected |
| | Spondweni | SM-6 V-1s | * Partly detected |
| Bunyaviridae | La Crosse | EVAg stocks, | Not detected |
| | | NC_004108 | |
| | Rift Valley Fever | H85/09 | Not detected |
| | Oropouche | EVAg stocks, | Not detected |
| | | 005v-EVA832 | |
| Alphaviridae | Chikingunya | — | Not detected |
| | Mayaro | TC652 | Not detected |
| | O'nyong'nyong | Ang'mom | Not detected |

RPA data generated using exemplary Zika RPA Fw, Zika RPA Rev and Zika RPA Probe 1 of the invention.
Spondweni virus, which belongs to the Spondweni serogroup together with Zika virus, was weakly detected at high template RNA concentration.

Robust Detection—Effect of Crude Samples

Existing RT-PCR based methods require intensive sample processing work, and are typically highly sensitive to sample quality. This increases sample preparation time and complexity, and typically requires the involvement of skilled technicians. The inventors therefore sought to provide a method that is more robust, and less sensitive to sample quality.

a) Testing the Inhibitory Effect of Using Dilutions of Crude Samples on the RPA Assay, Performed Using Primers and Probe of the Invention

The inventors tested the performance of the RPA assay, carried out using the primers and probe of the invention, in the presence of crude samples (serum, urine and mosquito homogenate). 5×10⁶ copies/reaction synthetic RNA template (South American outbreak strain) was tested, in the presence of a dilution series of crude samples. As shown in Table 8 and FIG. 8, the invention tolerates crude sample preparations very well.

TABLE 8

| Copies/μl template | Serum Ct | Urine Ct | Mosquito homogenate |
|---|---|---|---|
| (5 μl added/reaction) | (2 replicates) | (2 replicates) | Ct (2 replicates) |
| Neat | 6.2, 6.4, 6.5 | 6.3, 6.3, 6.9 | 8.4, 9.0, 9.8 |
| 1 in 10 | 6.0, 6.0, 6.1 | 6.2, 6.3, 7.0 | 7.9, 8.2, 8.3 |
| 1 in 100 | 6.0, 6.5, 7.0 | 6.2, 6.3, 6.3 | 6.1, 6.5, 6.8 |
| 1 in 1000 | 6.5, 6.8, 6.8 | 6.2, 6.2, 7.1 | 6.1, 6.1, 8.4 |
| 1 in 10⁴ | 6.3, 6.4, 6.5 | 6.1, 6.1, 7.9 | 7.0, 7.8, 7.9 |
| 1 in 10⁵ | 6.3, 6.7, 6.8 | 6.1, 6.3, 7.7 | 6.2, 7.0, 7.5 |
| 1 in 10⁶ | 6.1, 6.3, 6.3 | 5.9, 6.8, 7.0 | 6.1, 7.5, 7.6 |
| 1 in 10⁷ | 5.9, 6.1, 6.7 | 6.0, 6.1, 6.2 | 6.0, 7.0, 7.3 |

| | |
|---|---|
| PTC | 6.0, 6.0, 6.2 |
| NTC | 0, 0, 0 |

RPA data generated using exemplary Zika RPA 7 Fw and Zika RPA 11 Rev primers & Zika RPA Probe 1 of the invention.

b) Effect of Crude Samples on Assay Sensitivity

The inventors also tested the sensitivity of the RPA assay, carried out using the primers and probe of the invention, in the presence of crude samples (serum, urine and mosquito homogenate). 1 in 10 diluted crude samples added to a dilution series of synthetic RNA template (South American outbreak strain).

As shown in Table 9 (raw output data provided in FIG. 9), the inhibitory effect of crude samples on sensitivity of the assay is minimal.

TABLE 9

| | | | Mosquito | |
|---|---|---|---|---|
| Copies/μl template | Serum | Urine | homogenate | No crude |
| (5 μl added/reaction) | (Ct) | (Ct) | (Ct) | material |
| 10⁶ | 5, 5.1, 6.3 | 4.5, 4.5, 5.1 | 4.5, 4.5, 4.5 | 4.5, 4.5, 5.0 |
| 10⁵ | 6.0, 7.0, 7.0 | 5.0, 6.1, 6.3 | 5.0, 5.0, 5.1 | 5.7, 5.7, 6.1 |
| 10⁴ | 6.9, 7.0, 9.2 | 6.0, 6.2, 9.5 | 6.9, 8.2, 10.3 | 6.1, 6.1, 8.8 |
| 1000 | 10.4, 11.5, 11.5 | 8.8, 9.8, 10.9 | 9.7, 9.8, 10.1 | 8.1, 9.0, 9.0 |
| 100 | 0, 0, 33.0 | 16.0, 16.0, 16.0 | 21.5, 22, 24.2 | 20, 20, 22.5 |
| 10 | 0, 0, 0 | 0, 0, 0 | 0, 0, 0 | 0, 0, 0 |
| NTC | 0, 0, 0 | 0, 0, 0 | 0, 0, 0 | 0, 0, 0 |

RPA data generated using exemplary Zika RPA 7 Fw and Zika RPA 11 Rev primers & Zika RPA Probe 1 of the invention.

The addition of 1 in 10 diluted human serum shows a small inhibitory effect, with a 10-fold reduction in assay sensitivity. The 1 in 10 diluted mosquito larvae and synthetic urine standard have had little-to-no impact on the detection limit for this assay. These data demonstrate that the primers and probes of the invention are suitable for the testing of crude patient and insect samples with minimal sample preparation, particularly when used in the RPA assay.

The inventors also tested the sensitivity of the RPA assay, carried out using primers and probe of the invention, in the presence of further crude samples (semen, urine, saliva, serum and homogenised pooled mosquito samples). Crude semen, urine, saliva, serum samples were spiked into the reaction at a 1 in 10 and 1 in 100 dilution; and crude homogenised pooled mosquito samples were tested at a 1 in 1000 or 1 in 10,000 dilution. This was performed with extracted virus (South American strain—equivalent to 1×10² PFU for the testing of the bodily fluids at all dilutions and 5×10⁻¹ PFU for the 1 in 1000 dilution of the mosquito samples and 5×10⁻² PFU for the 1 in 10,000 dilution of the mosquito samples). As shown in FIG. 10, saliva samples were well tolerated, with no apparent inhibition at either 1 in 10 or 1 in 100 dilutions, crude serum showed good detection at a 1 in 100 dilution, urine showed some inhibition but target was still detected at both 1 in 10 and 1 in 100 dilutions.

Advantageously, no assay inhibition was apparent in the presence of mosquito homogenate, confirming the high suitability of the invention for use in an “in field” diagnostic assay.

Detection of Zika Virus Nucleic Acid in Clinical Samples

The inventors also tested the RPA assay, carried out using the primers and probe of the invention using a panel of clinical samples. Table 10 shows TTP signal obtained using the primers and probe of the invention, for a range of patient samples, including semen and urine, and is compared to that generated by RT-PCR.

Advantageously, the RPA assay, carried out using the primers and probe of the invention did not identify any false positives. Moreover, the vast majority of samples which were identified as positive by RT-PCR were also identified using the RPA assay, carried out using the primers and probe of the invention.

TABLE 10

| Sample | Sample | RT-RPA TTP | RT-RPA | RT-PCR |
|---|---|---|---|---|
| No | description | (mins) | result | result |
| 1 | Other clinical | 13.871 | + | + |
| 2 | Other clinical | 34.799 | + | + |
| 3 | Other clinical | 6.298 | + | + |
| 4 | Other clinical | 15.520 | + | + |
| 5 | Other clinical | 7.731 | + | + |
| 6 | Semen | 37.507 | + | + |
| 7 | Semen | 16.978 | + | + |
| 8 | Semen | 18.159 | + | + |
| 9 | Semen | Not detected | − | + |
| 10 | Semen | 6.399 | + | + |
| 11 | Semen | Not detected | − | + |
| 12 | Semen | 5.155 | + | + |
| 13 | Semen | Not detected | − | − |
| 14 | Urine | Not detected | − | + |
| 15 | Urine | Not detected | − | − |
| 16 | Urine | 19.318 | + | + |
| 17 | Urine | 22.508 | + | + |
| 18 | Urine | 34.593 | + | + |
| 19 | Urine | Not detected | − | − |
| 20 | Urine | Not detected | − | − |
| 21 | Urine | 15.293 | + | + |
| 22 | Urine | 16.227 | + | + |
| 23 | Urine | Not detected | − | + |
| 24 | Urine | 33.070 | + | + |
| 25 | Urine | Not detected | − | − |
| 26 | Serum | Not detected | − | − |
| 27 | Serum | Not detected | − | − |
| 28 | Serum | Not detected | − | − |

RPA data generated using exemplary Zika RPA Fw, Zika RPA Rev and Zika RPA Probe 1 of the invention.

Materials and Methods

Primer and Probe Preparation

Primers were prepared by Integrated DNA Technologies (IDT) and an RPA EXO probe by ATD BIO; all as HPLC purified material (see Table 1). Primer and probe stocks were prepared at 100 μM in a Tris-EDTA buffer and diluted to 10 μM in molecular grade dH20. A primer mix was prepared to 5 μM (both Fw and Rev primers) and both primer mix and probe stocks were frozen at −20 in single use aliquots.

Crude Sample Preparation

Human serum male AB (sigma H422-20ML) was aliquoted into single-use vials and stored at −20 C. Surine standard -ve control urine (Sigma S-020-50ML) was aliquoted and stored at fridge temperature. White mosquito larvae (Aquamarine fish food), was prepared by defrosting a single cube of larvae overnight at fridge temperature. 10 mosquito larvae were added to a Precellys-R tube with 300 μl of dH20, homogenised (3×20 sec, 30 sec break), then centrifuged for 5 minutes at 8000 rpm and the supernatant retained (neat sample). A 1 in 10 dilution series of serum, urine, and mosquito homogenate was prepared in molecular grade dH20 (10 μl into 90 μl).

Semen (Pooled Human Donors), urine (Pooled Human Donors) and saliva (Pooled Human Donors) were purchased from Lee Biosolutions, Inc. Serum (from 8 donors) was collected from volunteers at Public Health England and pooled.

Wild mosquitos were collected at Dee March, Merseyside, UK. Each mosquito was homogenised using a Precellys tissue homogeniser in 300 μL nuclease-free water using CK28-R 2 mL reinforced tubes at 2×20 seconds at 4000 rpm, with 30 second breaks. Homogenised samples were centrifuged at 8000 rpm for 5 min and cleared supernatant was removed to a fresh nuclease-free microcentrifuge tube. Mosquito homogenates were then pooled and aliquoted out for individual use.

RNA Template Preparation

Synthetic DNA fragments of 5 of the current Zika outbreak strains were prepared by IDT (KU365780-position 1155-2980, EU545988-position 1048-2873, KJ776791-position 1095-2920, KU321639-position 1153-2978 and KU365778-position 1142-2967)) with the addition of T7 and SP6 promoters at the 5′ and 3′ end respectively (see FIG. 11 for an example fragment). An RNA template was created using a T7 High Yield RNA synthesis kit (NEB). Approximately 1 μg of DNA was added per reaction to an 0.2 ml PCR tube with 2 μl each of 100 mM ATP, GTP, UTP and CTP, 2 μl RNA polymerase mix, 2 μl 10× reaction buffer and sufficient molecular grade dH20 to make the reaction up to 20 μl. The reaction was incubated at 37 degrees for two hours in a thermocycler. The RNA template was then DNAse-treated to remove the original template contamination; 70 μl nuclease free dH₂O was added per tube with 10 μl 10×DNAse I buffer and 2 μl RNAse-free DNAse I (NEB). The tubes were mixed and incubated for 15 mins at 37 degrees. The RNA was purified using a Qiagen RNeasy minikit and quantified using a Qubit broad range RNA kit (Thermo-Fisher Scientific).

Viral Sample Preparation

Two Zika viral strains, African Zika virus (Strain MP1751, NCBI accession number KY288905, from Uganda, 1962) and South American Zika virus (Strain PRVABC59, NCBI accession number KU501215, from Puerto Rico, 2015) were cultured and viral RNA was extracted using QIAmp viral RNA kit (QIAgen).

EXO RT-RPA Method

The Exo RT-RPA was performed in a 50 μl volume using a TwistAmp Exo-RT kit (TwistDx Cambridge UK). A mastermix was prepared, composed of the following/reaction; 4.2 μl of the 5 μM primer mix, 0.6 μl of the 10 μM Exo probe, 29.5 μl rehydration buffer and sufficient distilled water to make the reaction up to 50 μl after addition of all assay components. Where crude samples were used, 20 units (0.5 μl) of an RNAse inhibitor was also included (RNAseOUT 40 U/μl Invitrogen). The mastermix was distributed into the wells of a 96-well PCR plate. 1-5 μl of template (together with 5 μl crude sample if used) was added and the reaction mixture combined with the lyophilised enzyme pellet, before returning to the plate. The Magnesium acetate was diluted to 140 mM with molecular grade dH₂O and 5 μl was added last and the plate briefly centrifuged before running for 40 degrees for 40 minutes on an ABI 7500 real-time PCR system, with fluorescence detection every 60 seconds in the FAM channel. Alternatively, the assay was run on a QuantStudio Flex 7 real-time PCR machine (Thermo-Fisher Scientific) with fluorescence detection every 60 sec in the FAM channel, without ROX passive reference.

RT-PCR

Method adapted from Lanciotti et al. (2008). Briefly, each reaction was performed using 0.9 μM of ZIKV 1086 forward primer, 0.9 μM of ZIKV 1162c reverse primer, 1 μM of ZIKV 1107-FAM probe, 0.8 μL of SuperScript III Taq and 10 μL of 2× Reaction buffer from SuperScript III Platinum One-Step qRT-PCR kit (Thermo-Fisher Scientific), 0.5 mM MgSO₄, 5 μL of template and sufficient volume of nuclease-free water to achieve 20 μL total volume. The reverse transcriptase step was performed at 50° C. for 10 min, followed by denaturation at 95° C. for 2 min and amplification stage consisting of 45 cycles of denaturation of 95° C. for 10 sec and annealing/extension at 60° C. for 40 min. Fluorescence was detected in the FAM channel during the extension step of each cycle without ROX passive reference.

The inventors note that TTP (Time to positive/time to threshold, measured in minutes) data and Ct (cycle number at which the threshold is reached) data for RT-RPA are equivalent, i.e. 1 min=1 cycle. For RT-PCR, TTP=(Ct×1.32 min/cycle)+12 mins.

RPA with an RT-RPA Basic Kit

The RT-RPA basic assay was performed in a 50 μl volume using a TwistAmp Basic-RT kit (TwistDx Cambridge UK). A mastermix was prepared, composed of the following/reaction; 4.2 μl of 5 μM primer mix, 29.5 μl rehydration buffer and sufficient distilled water to make the reaction up to 50 μl after addition of template. The mastermix was distributed into 0.2 ml PCR tube strips. 5 μl of template was added and the reaction mixture combined with the lyophilised enzyme pellet, before returning to the wells. The Magnesium acetate was diluted to 140 mM with molecular grade dH₂0 and 5 μl was added last. The tube strip was then briefly centrifuged before incubating for 40 degrees for 40 minutes on a thermocycler. The products of the RPA were purified using a QIAgen QIAquick PCR purification kit, then run on an Invitrogen 1% Agarose gel (E-Gel EX with Sybr Gold II) with an Invitrogen E gel 1 Kb plus ladder.

1. A composition comprising:
a) a nucleic acid probe comprising:
(i) nucleic acid sequence of any one of SEQ ID NOs: 31-114; or
(ii) nucleic acid sequence exhibiting at least 85% identity to any one of SEQ ID NOs: 31-114; or
(iii) nucleic acid sequence comprising 15 or more consecutive nucleic acids of the nucleic acid sequence of SEQ ID NOs: 31-114;
wherein: group “3”=modification that functions to block polymerase extension;
group “5”=dT-fluorophore;
group “6”=an abasic nucleotide analog; and
group “7”=dT-quencher group (suitable for group “5”);
and/or
b) a forward nucleic acid primer and a reverse nucleic acid primer; the forward nucleic acid primer comprising:
i) nucleic acid sequence GTGAAGCCTACCTTG (SEQ ID NO: 2); or
ii) nucleic acid sequence exhibiting at least 85% identity to the nucleic acid sequence of SEQ ID NO: 2;
and the reverse nucleic acid primer comprising:
i) nucleic acid sequence TGGATGCTCTTCCCG (SEQ ID NO: 17); or ii) nucleic acid sequence exhibiting at least 85% identity to the nucleic acid sequence of SEQ ID NO: 17.

2. The composition of claim 1, wherein:
group “3”=selected from (i) C3-spacer, (ii) a phosphate, (iii) a biotin-TEG, or (iv) an amine;
group “5”=selected from (i) dT-fluorescein, (ii) TAMRA and (iii) Cy5;
group “6”=D-spacer; and
group “7”=selected from (i) dT-BHQ1 and (ii) dT-BHQ2.

3. The composition of claim 2, wherein:
group “3”=propanol;
group “5”=dT-fluorescein;
group “6”=D-spacer; and
group “7”=dT-BHQ1.

4. The composition of claim 1, wherein the forward nucleic acid primer comprises or consists of:
(i) the nucleic acid sequence of any one of SEQ ID NOs: 1-15;
(ii) nucleic acid sequence exhibiting at least 85% identity to the nucleic acid sequence of any one of SEQ ID NOs: 1-15; or
(iii) nucleic acid sequence comprising 15 or more consecutive nucleic acids of the nucleic acid sequence of any one of SEQ ID NOs: 1-15.

5. The composition of claim 1, wherein the reverse nucleic acid primer comprises or consists of:
(i) the nucleic acid sequence of any one of SEQ ID NOs: 16-30;
(ii) nucleic acid sequence exhibiting at least 85% identity to the nucleic acid sequence of any one of SEQ ID NOs: 16-30; or
(iii) nucleic acid sequence comprising 15 or more consecutive nucleic acids of any one of SEQ ID NOs: 16-30.

6. The composition of claim 1, comprising:
(i) forward nucleic acid primer comprising or consisting of the nucleic acid sequence of SEQ ID NO: 3, and/or exhibiting at least 85% identity to the nucleic acid sequence of any one of SEQ ID NO: 3;
(ii) reverse nucleic acid primer comprising or consisting of the nucleic acid sequence of SEQ ID NO: 18, and/or exhibiting at least 85% identity to the nucleic acid sequence of any one of SEQ ID NO: 18; and
(iii) nucleic acid probe comprising or consisting of the nucleic acid sequence of SEQ ID NO: 31, and/or exhibiting at least 85% identity to the nucleic acid sequence of any one of SEQ ID NO: 31.

7. The composition of claim 1, comprising:
(i) forward nucleic acid primer comprising or consisting of the nucleic acid sequence of SEQ ID NO: 1 and/or exhibiting at least 85% identity to the nucleic acid sequence of any one of SEQ ID NO: 1;
(ii) reverse nucleic acid primer comprising or consisting of the nucleic acid sequence of SEQ ID NO: 16, and/or exhibiting at least 85% identity to the nucleic acid sequence of any one of SEQ ID NO: 16; and
(iii) nucleic acid probe comprising or consisting of the nucleic acid sequence of SEQ ID NO: 31, and/or exhibiting at least 85% identity to the nucleic acid sequence of any one of SEQ ID NO: 31.

8. A kit comprising a composition according to claim 1.

9. A device comprising a composition according to claim 1.

10. The kit of claim 8, comprising part or whole of a TwistAmp Basic kit, TwistAmp Basic-RT kit, TwistAmp exo kit, TwistAmp exo-RT kit, TwistAmp fpg kit, and/or TwistAmp nfo kit.

11. Use of a composition of claim 1 in a method of detecting Zika virus in a sample.

12. A method for detecting the presence of Zika virus in a sample or detecting the absence of said Zika virus in said sample, said method comprising:
A) combining said sample with a composition according to claim 1;
B) allowing nucleic acid present in the sample to contact the primers and/or probes within the composition; and
C) performing a nucleic acid amplification technique;
wherein amplification of nucleic acid in the sample confirms that nucleic acid from Zika virus is present within the sample, and wherein the absence of amplification of nucleic acid in the sample confirms that nucleic acid from Zika virus is absent from the sample.

13. The method of claim 12, wherein the nucleic acid amplification technique is an isothermal nucleic acid amplification technique.

14. The method of claim 13, wherein the isothermal nucleic acid amplification technique is Recombinase Polymerase Amplification.

15. The method of claim 12, wherein the sample is from an individual, typically an animal.

16. The method of claim 15, wherein the animal is a mammal, typically a human.

17. The method of claim 16, wherein the sample is selected from blood, plasma, saliva, serum, sputum, urine, cerebral spinal fluid, semen, cells, a cellular extract, a tissue sample, a tissue biopsy, a stool sample, a swab from any body site and/or one or more organs; typically blood, serum, urine, saliva and/or organ(s).

18. The method of claim 15, wherein the animal is an insect, typically a mosquito, typically an Aedes mosquito.

19. The method of claim 18, wherein the sample is homogenised mosquito(s).

20. The method of claim 12, wherein the sample is a crude sample.

21. The method of claim 12, for use in surveillance of Zika virus prevalence.

22. The method of claim 12, for use in diagnosing Zika virus infection in an individual.

23. The method of claim 22 wherein, upon identification of Zika virus infection in the individual, said individual is provided with an appropriate treatment or therapy.


SEQUENCE LISTING (120 sequences)

SEQ ID NO: 1 - DNA, 35 bp, Artificial
  Synthetic
ccaacacaaggtgaagcctaccttgacaagcaatc

SEQ ID NO: 2 - DNA, 15 bp, Artificial
  Synthetic
gtgaagcctaccttg

SEQ ID NO: 3 - DNA, 34 bp, Artificial
  Synthetic
ccaacacaaggtgaagcctaccttgacaagcaat

SEQ ID NO: 4 - DNA, 35 bp, Artificial
  Synthetic
cccaacacaaggtgaagcctaccttgacaagcaat

SEQ ID NO: 5 - DNA, 35 bp, Artificial
  Synthetic
gcccaacacaaggtgaagcctaccttgacaagcaa

SEQ ID NO: 6 - DNA, 35 bp, Artificial
  Synthetic
tgcccaacacaaggtgaagcctaccttgacaagca

SEQ ID NO: 7 - DNA, 35 bp, Artificial
  Synthetic
ctgcccaacacaaggtgaagcctaccttgacaagc

SEQ ID NO: 8 - DNA, 35 bp, Artificial
  Synthetic
gctgcccaacacaaggtgaagcctaccttgacaag

SEQ ID NO: 9 - DNA, 35 bp, Artificial
  Synthetic
cgctgcccaacacaaggtgaagcctaccttgacaa

SEQ ID NO: 10 - DNA, 35 bp, Artificial
  Synthetic
caacacaaggtgaagcctaccttgacaagcaatca

SEQ ID NO: 11 - DNA, 35 bp, Artificial
  Synthetic
aacacaaggtgaagcctaccttgacaagcaatcag

SEQ ID NO: 12 - DNA, 35 bp, Artificial
  Synthetic
acacaaggtgaagcctaccttgacaagcaatcaga

SEQ ID NO: 13 - DNA, 35 bp, Artificial
  Synthetic
cacaaggtgaagcctaccttgacaagcaatcagac

SEQ ID NO: 14 - DNA, 35 bp, Artificial
  Synthetic
acaaggtgaagcctaccttgacaagcaatcagaca

SEQ ID NO: 15 - DNA, 35 bp, Artificial
  Synthetic
caaggtgaagcctaccttgacaagcaatcagacac

SEQ ID NO: 16 - DNA, 35 bp, Artificial
  Synthetic
attctctggctggatgctcttcccggtcattttct

SEQ ID NO: 17 - DNA, 15 bp, Artificial
  Synthetic
tggatgctcttcccg

SEQ ID NO: 18 - DNA, 33 bp, Artificial
  Synthetic
ttctctggctggatgctcttcccggtcattttc

SEQ ID NO: 19 - DNA, 35 bp, Artificial
  Synthetic
aattctctggctggatgctcttcccggtcattttc

SEQ ID NO: 20 - DNA, 35 bp, Artificial
  Synthetic
caattctctggctggatgctcttcccggtcatttt

SEQ ID NO: 21 - DNA, 35 bp, Artificial
  Synthetic
ccaattctctggctggatgctcttcccggtcattt

SEQ ID NO: 22 - DNA, 35 bp, Artificial
  Synthetic
tccaattctctggctggatgctcttcccggtcatt

SEQ ID NO: 23 - DNA, 35 bp, Artificial
  Synthetic
ctccaattctctggctggatgctcttcccggtcat

SEQ ID NO: 24 - DNA, 35 bp, Artificial
  Synthetic
gctccaattctctggctggatgctcttcccggtca

SEQ ID NO: 25 - DNA, 35 bp, Artificial
  Synthetic
ttctctggctggatgctcttcccggtcattttctc

SEQ ID NO: 26 - DNA, 35 bp, Artificial
  Synthetic
tctctggctggatgctcttcccggtcattttctct

SEQ ID NO: 27 - DNA, 35 bp, Artificial
  Synthetic
ctctggctggatgctcttcccggtcattttctctg

SEQ ID NO: 28 - DNA, 35 bp, Artificial
  Synthetic
tctggctggatgctcttcccggtcattttctctgg

SEQ ID NO: 29 - DNA, 35 bp, artificial
  Synthetic
ctggctggatgctcttcccggtcattttctctgga

SEQ ID NO: 30 - DNA, 35 bp, Artificial
  Synthetic
tggctggatgctcttcccggtcattttctctggag

SEQ ID NO: 31 - DNA, 50 bp, Artificial
  Synthetic
  misc_feature (33)..(33): n is dT-fluorophore
  misc_feature (34)..(34): n is an abasic nucleotide
  misc_feature (35)..(35): n is dT-quencher group
  misc_feature (50)..(50): wherein the A at position 50 is modified to block polymerase extension
gaacgttagtggacagaggctggggaaatggannnggactttttggcaaa

SEQ ID NO: 32 - DNA, 20 bp, Artificial
  Synthetic
  misc_feature (17)..(17): n is dT-fluorophore
  misc_feature (18)..(18): n is an abasic nucleotide
  misc_feature (19)..(19): n is dT-quencher group
  misc_feature (20)..(20): wherein the G at position 20 is modified to block polymerase extension
aggctggggaaatggannng

SEQ ID NO: 33 - DNA, 20 bp, Artificial
  Synthetic
  misc_feature (1)..(1): n is dT-fluorophore
  misc_feature (2)..(2): n is an abasic nucleotide
  misc_feature (3)..(3): n is dT-quencher group
  misc_feature (20)..(20): wherein the G at position 20 is moified to block polymerase extension
nnnctggggaaatggatgtg

SEQ ID NO: 34 - DNA, 20 bp, Artificial
  Synthetic
  misc_feature (2)..(2): n is dT-fluorophore
  misc_feature (3)..(3): n is an abasic nucleotide
  misc_feature (4)..(4): n is dT-quencher group
  misc_feature (20)..(20): wherein the G at position 20 is modified to block polymerase extension
annntggggaaatggatgtg

SEQ ID NO: 35 - DNA, 20 bp, Artificial
  Synthetic
  misc_feature (3)..(3): n is dT-fluorophore
  misc_feature (4)..(4): n is an abasic nucleotide
  misc_feature (5)..(5): n is dT-quencher group
  misc_feature (20)..(20): wherein the G at position 20 is modified to block polymerase extension
agnnnggggaaatggatgtg

SEQ ID NO: 36 - DNA, 20 bp, Artificial
  Synthetic
  misc_feature (4)..(4): n is dT-fluorophore
  misc_feature (5)..(5): n is an abasic nucleotide
  misc_feature (6)..(6): n is dT-quencher group
  misc_feature (20)..(20): wherein the G at position 20 is modified to block polymerase extension
aggnnngggaaatggatgtg

SEQ ID NO: 37 - DNA, 20 bp, Artificial
  Synthetic
  misc_feature (5)..(5): n is dT-fluorophore
  misc_feature (6)..(6): n is an abasic nucleotide
  misc_feature (7)..(7): n is dT-quencher group
  misc_feature (20)..(20): wherein the G at position 20 is modified to block polymerase extension
aggcnnnggaaatggatgtg

SEQ ID NO: 38 - DNA, 20 bp, Artificial
  Synthetic
  misc_feature (6)..(6): n is dT-fluorophore
  misc_feature (7)..(7): n is an abasic nucleotide
  misc_feature (8)..(8): n is dT-quencher group
  misc_feature (20)..(20): wherein the G at position 20 is modified to block polymerase extension
aggctnnngaaatggatgtg

SEQ ID NO: 39 - DNA, 20 bp, Artificial
  Synthetic
  misc_feature (7)..(7): n is dT-fluorophore
  misc_feature (8)..(8): n is an abasic nucleotide
  misc_feature (9)..(9): n is dT-quencher group
  misc_feature (20)..(20): wherein the G at position 20 is modified to block polymerase extension
aggctgnnnaaatggatgtg

SEQ ID NO: 40 - DNA, 20 bp, Artificial
  Synthetic
  misc_feature (8)..(8): n is dT-fluorophore
  misc_feature (9)..(9): n is an abasic nucleotide
  misc_feature (10)..(10): n is dT-quencher group
  misc_feature (20)..(20): wherein the G at position 20 is modified to block polymerase extension
aggctggnnnaatggatgtg

SEQ ID NO: 41 - DNA, 20 bp, Artificial
  Synthetic
  misc_feature (9)..(9): n is dT-fluorophore
  misc_feature (10)..(10): n is an abasic nucleotide
  misc_feature (11)..(11): n is dT-quencher group
  misc_feature (20)..(20): wherein the G at position 20 is modified to block polymerase extension
aggctgggnnnatggatgtg

SEQ ID NO: 42 - DNA, 20 bp, Artificial
  Synthetic
  misc_feature (10)..(10): n is dT-fluorophore
  misc_feature (11)..(11): n is an abasic nucleotide
  misc_feature (12)..(12): n is dT-quencher group
  misc_feature (20)..(20): wherein the G at position 20 is modified to block polymerase extension
aggctggggnnntggatgtg

SEQ ID NO: 43 - DNA, 20 bp, Artificial
  Synthetic
  misc_feature (11)..(11): n is dT-fluorophore
  misc_feature (12)..(12): n is an abasic nucleotide
  misc_feature (13)..(13): n is dT-quencher group
  misc_feature (20)..(20): wherein the G at position 20 is modified to block polymerase extension
aggctggggannnggatgtg

SEQ ID NO: 44 - DNA, 20 bp, Artificial
  Synthetic
  misc_feature (12)..(12): n is dT-fluorophore
  misc_feature (13)..(13): n is an abasic nucleotide
  misc_feature (14)..(14): n is dT-quencher group
  misc_feature (20)..(20): wherein the G at position 20 is modified to block polymerase extension
aggctggggaannngatgtg

SEQ ID NO: 45 - DNA, 20 bp, Artificial
  Synthetic
  misc_feature (13)..(13): n is dT-fluorophore
  misc_feature (14)..(14): n is an abasic nucleotide
  misc_feature (15)..(15): n is dT-quencher group
  misc_feature (20)..(20): wherein the G at position 20 is modified to block polymerase extension
aggctggggaaannnatgtg

SEQ ID NO: 46 - DNA, 20 bp, Artificial
  Synthetic
  misc_feature (14)..(14): n is dT-fluorophore
  misc_feature (15)..(15): n is an abasic nucleotide
  misc_feature (16)..(16): n is dT-quencher group
  misc_feature (20)..(20): wherein the G at position 20 is modified to block polymerase extension
aggctggggaaatnnntgtg

SEQ ID NO: 47 - DNA, 20 bp, Artificial
  Synthetic
  misc_feature (15)..(15): n is dT-fluorophore
  misc_feature (16)..(16): n is an abasic nucleotide
  misc_feature (17)..(17): n is dT-quencher group
  misc_feature (20)..(20): wherein the G at position 20 is modified to block polymerase extension
aggctggggaaatgnnngtg

SEQ ID NO: 48 - DNA, 20 bp, Artificial
  Synthetic
  misc_feature (16)..(16): n is dT-fluorophore
  misc_feature (17)..(17): n is an abasic nucleotide
  misc_feature (18)..(18): n is dT-quencher group
  misc_feature (20)..(20): wherein the G at position 20 is modified to block polymerase extension
aggctggggaaatggnnntg

SEQ ID NO: 49 - DNA, 20 bp, Artificial
  Synthetic
  misc_feature (17)..(17): n is dT-fluorophore
  misc_feature (18)..(18): n is an abasic nucleotide
  misc_feature (19)..(19): n is dT-quencher group
  misc_feature (20)..(20): wherein the G at position 20 is modified to block polymerase extension
aggctggggaaatggannng

SEQ ID NO: 50 - DNA, 20 bp, Artificial
  Synthetic
  misc_feature (18)..(18): n is dT-fluorophore
  misc_feature (19)..(19): n is an abasic nucleotide
  misc_feature (20)..(20): n at position 20 is is dT-quencher group which is modified to block polymerase extension
aggctggggaaatggatnnn

SEQ ID NO: 51 - DNA, 50 bp, Artificial
  Synthetic
  misc_feature (34)..(34): n is dT-fluorophore
  misc_feature (35)..(35): n is an abasic nucleotide
  misc_feature (36)..(36): n is dT-quencher group
  misc_feature (50)..(50): wherein the A at position 50 is modified to block polymerase extension
agaacgttagtggacagaggctggggaaatggannnggactttttggcaa

SEQ ID NO: 52 - DNA, 50 bp, Artificial
  Synthetic
  misc_feature (35)..(35): n is dT-fluorophore
  misc_feature (36)..(36): n is an abasic nucleotide
  misc_feature (37)..(37): n is dT-quencher group
  misc_feature (50)..(50): wherein the A at position 50 is modified to block polymerase extension
aagaacgttagtggacagaggctggggaaatggannnggactttttggca

SEQ ID NO: 53 - DNA, 50 bp, Artificial
  Synthetic
  misc_feature (36)..(36): n is dT-fluorophore
  misc_feature (37)..(37): n is an abasic nucleotide
  misc_feature (38)..(38): n is dT-quencher group
  misc_feature (50)..(50): wherein the C at position 50 is modified to block polymerase extension
aaagaacgttagtggacagaggctggggaaatggannnggactttttggc

SEQ ID NO: 54 - DNA, 50 bp, Artificial
  Synthetic
  misc_feature (37)..(37): n is dT-fluorophore
  misc_feature (38)..(38): n is an abasic nucleotide
  misc_feature (39)..(39): n is dT-quencher group
  misc_feature (50)..(50): wherein the G at position 50 is modified to block polymerase extension
aaaagaacgttagtggacagaggctggggaaatggannnggactttttgg

SEQ ID NO: 55 - DNA, 50 bp, Artificial
  Synthetic
  misc_feature (38)..(38): n is dT-fluorophore
  misc_feature (39)..(39): n is an abasic nucleotide
  misc_feature (40)..(40): n is dT-quencher group
  misc_feature (50)..(50): wherein the G at position 50 is modified to block polymerase extension
caaaagaacgttagtggacagaggctggggaaatggannnggactttttg

SEQ ID NO: 56 - DNA, 50 bp, Artificial
  Synthetic
  misc_feature (39)..(39): n is dT-fluorophore
  misc_feature (40)..(40): n is an abasic nucleotide
  misc_feature (41)..(41): n is dT-quencher group
  misc_feature (50)..(50): wherein the T at position 50 is modified to block polymerase extension
gcaaaagaacgttagtggacagaggctggggaaatggannnggacttttt

SEQ ID NO: 57 - DNA, 50 bp, Artificial
  Synthetic
  misc_feature (32)..(32): n is dT-fluorophore
  misc_feature (33)..(33): n is an abasic nucleotide
  misc_feature (34)..(34): n is dT-quencher group
  misc_feature (50)..(50): wherein the G at position 50 is modified to block polymerase extension
aacgttagtggacagaggctggggaaatggannnggactttttggcaaag

SEQ ID NO: 58 - DNA, 50 bp, Artificial
  Synthetic
  misc_feature (31)..(31): n is dT-fluorophore
  misc_feature (32)..(32): n is an abasic nucleotide
  misc_feature (33)..(33): n is dT-quencher group
  misc_feature (50)..(50): wherein the G at position 50 is modified to block polymerase extension
acgttagtggacagaggctggggaaatggannnggactttttggcaaagg

SEQ ID NO: 59 - DNA, 50 bp, Artificial
  Synthetic
  misc_feature (30)..(30): n is dT-fluorophore
  misc_feature (31)..(31): n is an abasic nucleotide
  misc_feature (32)..(32): n is dT-quencher group
  misc_feature (50)..(50): wherein the G at position 50 is modified to block polymerase extension
cgttagtggacagaggctggggaaatggannnggactttttggcaaaggg

SEQ ID NO: 60 - DNA, 50 bp, Artificial
  Synthetic
  misc_feature (29)..(29): n is dT-fluorophore
  misc_feature (30)..(30): n is an abasic nucleotide
  misc_feature (31)..(31): n is dT-quencher group
  misc_feature (50)..(50): wherein the A at position 50 is modified to block polymerase extension
gttagtggacagaggctggggaaatggannnggactttttggcaaaggga

SEQ ID NO: 61 - DNA, 50 bp, Artificial
  Synthetic
  misc_feature (28)..(28): n is dT-fluorophore
  misc_feature (29)..(29): n is an abasic nucleotide
  misc_feature (30)..(30): n is dT-quencher group
  misc_feature (50)..(50): wherein the G at position 50 is modified to block polymerase extension
ttagtggacagaggctggggaaatggannnggactttttggcaaagggag

SEQ ID NO: 62 - DNA, 50 bp, Artificial
  Synthetic
  misc_feature (27)..(27): n is dT-fluorophore
  misc_feature (28)..(28): n is an abasic nucleotide
  misc_feature (29)..(29): n is dT-quencher group
  misc_feature (50)..(50): wherein the C at position 50 is modified to block polymerase extension
tagtggacagaggctggggaaatggannnggactttttggcaaagggagc

SEQ ID NO: 63 - DNA, 22 bp, Artificial
  Synthetic
  misc_feature (6)..(6): n is dT-fluorophore
  misc_feature (7)..(7): n is an abasic nucleotide
  misc_feature (10)..(10): n is dT-quencher group
gaacgnnagnggacagaggctg

SEQ ID NO: 64 - DNA, 50 bp, Artificial
  Synthetic
  misc_feature (6)..(6): n is dT-fluorophore
  misc_feature (7)..(7): n is an abasic nucleotide
  misc_feature (10)..(10): n is dT-quencher group
gaacgnnagnggacagaggctggggaaatggatgtggactttttggcaaa

SEQ ID NO: 65 - DNA, 23 bp, Artificial
  Synthetic
  misc_feature (6)..(6): n is dT-fluorophore
  misc_feature (8)..(8): n is an abasic nucleotide
  misc_feature (10)..(10): n is dT-quencher group
gaacgntngnggacagaggctgg

SEQ ID NO: 66 - DNA, 50 bp, Artificial
  Synthetic
  misc_feature (6)..(6): n is dT-fluorophore
  misc_feature (8)..(8): n is an abasic nucleotide
  misc_feature (10)..(10): n is dT-quencher group
gaacgntngnggacagaggctggggaaatggatgtggactttttggcaaa

SEQ ID NO: 67 - DNA, 24 bp, Artificial
  Synthetic
  misc_feature (6)..(6): n is dT-fluorophore
  misc_feature (9)..(9): n is an abasic nucleotide
  misc_feature (10)..(10): n is dT-quencher group
gaacgntannggacagaggctggg

SEQ ID NO: 68 - DNA, 50 bp, Artificial
  Synthetic
  misc_feature (6)..(6): n is dT-fluorophore
  misc_feature (9)..(9): n is an abasic nucleotide
  misc_feature (10)..(10): n is dT-quencher group
gaacgntannggacagaggctggggaaatggatgtggactttttggcaaa

SEQ ID NO: 69 - DNA, 22 bp, Artificial
  Synthetic
  misc_feature (6)..(6): n is dT-quencher group
  misc_feature (7)..(7): n is an abasic nucleotide
  misc_feature (10)..(10): n is dT-fluorophore
gaacgnnagnggacagaggctg

SEQ ID NO: 70 - DNA, 50 bp, Artificial; identical to SEQ ID NO: 64
  Synthetic
  misc_feature (6)..(6): n is dT-quencher group
  misc_feature (7)..(7): n is an abasic nucleotide
  misc_feature (10)..(10): n is dT-fluorophore

SEQ ID NO: 71 - DNA, 23 bp, Artificial
  Synthetic
  misc_feature (6)..(6): n is dT-quencher group
  misc_feature (8)..(8): n is an abasic nucleotide
  misc_feature (10)..(10): n is dT-fluorophore
gaacgntngnggacagaggctgg

SEQ ID NO: 72 - DNA, 50 bp, Artificial; identical to SEQ ID NO: 66
  Synthetic
  misc_feature (6)..(6): n is dT-quencher group
  misc_feature (8)..(8): n is an abasic nucleotide
  misc_feature (10)..(10): n is dT-fluorophore

SEQ ID NO: 73 - DNA, 24 bp, Artificial
  Synthetic
  misc_feature (6)..(6): n is dT-quencher group
  misc_feature (9)..(9): n is an abasic nucleotide
  misc_feature (10)..(10): n is dT-fluorophore
gaacgntannggacagaggctggg

SEQ ID NO: 74 - DNA, 24 bp, Artificial
  Synthetic
  misc_feature (6)..(6): n is dT-quencher group
  misc_feature (9)..(9): n is an abasic nucleotide
  misc_feature (10)..(10): n is dT-fluorophore
gaacgntannggacagaggctggg

SEQ ID NO: 75 - DNA, 31 bp, Artificial
  Synthetic
  misc_feature (15)..(15): n is dT-fluorophore
  misc_feature (16)..(16): n is an abasic nucleotide
  misc_feature (19)..(19): n is dT-quencher group
agaggctggggaaanngangtggactttttg

SEQ ID NO: 76 - DNA, 50 bp, Artificial
  Synthetic
  misc_feature (29)..(29): n is dT-fluorophore
  misc_feature (30)..(30): n is an abasic nucleotide
  misc_feature (33)..(33): n is dT-quencher group
gaacgttagtggacagaggctggggaaanngangtggactttttggcaaa

SEQ ID NO: 77 - DNA, 31 bp, Artificial
  Synthetic
  misc_feature (14)..(14): n is dT-fluorophore
  misc_feature (16)..(16): n is an abasic nucleotide
  misc_feature (18)..(18): n is dT-quencher group
gaggctggggaaangnangtggactttttgg

SEQ ID NO: 78 - DNA, 50 bp, Artificial
  Synthetic
  misc_feature (29)..(29): n is dT-fluorophore
  misc_feature (31)..(31): n is an abasic nucleotide
  misc_feature (33)..(33): n is dT-quencher group
gaacgttagtggacagaggctggggaaangnangtggactttttggcaaa

SEQ ID NO: 79 - DNA, 31 bp, Artificial
  Synthetic
  misc_feature (13)..(13): n is dT-fluorophore
  misc_feature (16)..(16): n is an abasic nucleotide
  misc_feature (17)..(17): n is dT-quencher group
aggctggggaaanggnngtggactttttggc

SEQ ID NO: 80 - DNA, 50 bp, Artificial
  Synthetic
  misc_feature (29)..(29): n is dT-fluorophore
  misc_feature (32)..(32): n is an abasic nucleotide
  misc_feature (33)..(33): n is dT-quencher group
gaacgttagtggacagaggctggggaaanggnngtggactttttggcaaa

SEQ ID NO: 81 - DNA, 31 bp, Artificial; identical to SEQ ID NO: 75
  Synthetic
  misc_feature (15)..(15): n is dT-quencher group
  misc_feature (16)..(16): n is an abasic nucleotide
  misc_feature (19)..(19): n is dT-fluorophore

SEQ ID NO: 82 - DNA, 50 bp, Artificial; identical to SEQ ID NO: 76
  Synthetic
  misc_feature (29)..(29): n is dT-quencher group
  misc_feature (30)..(30): n is an abasic nucleotide
  misc_feature (33)..(33): n is dT-fluorophore

SEQ ID NO: 83 - DNA, 31 bp, Artificial; identical to SEQ ID NO: 77
  Synthetic
  misc_feature (14)..(14): n is dT-quencher group
  misc_feature (16)..(16): n is an abasic nucleotide
  misc_feature (18)..(18): n is dT-fluorophore

SEQ ID NO: 84 - DNA, 50 bp, Artificial; identical to SEQ ID NO: 78
  Synthetic
  misc_feature (29)..(29): n is dT-quencher group
  misc_feature (31)..(31): n is an abasic nucleotide
  misc_feature (33)..(33): n is dT-fluorophore

SEQ ID NO: 85 - DNA, 31 bp, Artificial; identical to SEQ ID NO: 79
  Synthetic
  misc_feature (13)..(13): n is dT-quencher group
  misc_feature (16)..(16): n is an abasic nucleotide
  misc_feature (17)..(17): n is dT-fluorophore

SEQ ID NO: 86 - DNA, 50 bp, Artificial; identical to SEQ ID NO: 80
  Synthetic
  misc_feature (29)..(29): n is dT-quencher group
  misc_feature (32)..(32): n is an abasic nucleotide
  misc_feature (33)..(33): n is dT-fluorophore

SEQ ID NO: 87 - DNA, 30 bp, Artificial
  Synthetic
  misc_feature (15)..(15): n is dT-fluorophore
  misc_feature (16)..(16): n is an abasic nucleotide
  misc_feature (20)..(20): n is dT-quencher group
tggggaaatggatgnngacnttttggcaaa

SEQ ID NO: 88 - DNA, 50 bp, Artificial
  Synthetic
  misc_feature (35)..(35): n is dT-fluorophore
  misc_feature (36)..(36): n is an abasic nucleotide
  misc_feature (40)..(40): n is dT-quencher group
gaacgttagtggacagaggctggggaaatggatgnngacnttttggcaaa

SEQ ID NO: 89 - DNA, 29 bp, Artificial
  Synthetic
  misc_feature (14)..(14): n is dT-fluorophore
  misc_feature (16)..(16): n is an abasic nucleotide
  misc_feature (19)..(19): n is dT-quencher group
ggggaaatggatgngnacnttttggcaaa

SEQ ID NO: 90 - DNA, 50 bp, Artificial
  Synthetic
  misc_feature (35)..(35): n is dT-fluorophore
  misc_feature (37)..(37): n is an abasic nucleotide
  misc_feature (40)..(40): n is dT-quencher group
gaacgttagtggacagaggctggggaaatggatgngnacnttttggcaaa

SEQ ID NO: 91 - DNA, 28 bp, Artificial
  Synthetic
  misc_feature (13)..(13): n is dT-fluorophore
  misc_feature (16)..(16): n is an abasic nucleotide
  misc_feature (18)..(18): n is dT-quencher group
gggaaatggatgnggncnttttggcaaa

SEQ ID NO: 92 - DNA, 50 bp, Artificial
  Synthetic
  misc_feature (35)..(35): n is dT-fluorophore
  misc_feature (38)..(38): n is an abasic nucleotide
  misc_feature (40)..(40): n is dT-quencher group
gaacgttagtggacagaggctggggaaatggatgnggncnttttggcaaa

SEQ ID NO: 93 - DNA, 27 bp, Artificial
  Synthetic
  misc_feature (12)..(12): n is dT-fluorophore
  misc_feature (16)..(16): n is an abasic nucleotide
  misc_feature (17)..(17): n is dT-quencher group
ggaaatggatgnggannttttggcaaa

SEQ ID NO: 94 - DNA, 50 bp, Artificial
  Synthetic
  misc_feature (35)..(35): n is dT-fluorophore
  misc_feature (39)..(39): n is an abasic nucleotide
  misc_feature (40)..(40): n is dT-quencher group
gaacgttagtggacagaggctggggaaatggatgnggannttttggcaaa

SEQ ID NO: 95 - DNA, 30 bp, Artificial; identical to SEQ ID NO: 87
  Synthetic
  misc_feature (15)..(15): n is dT-quencher group
  misc_feature (16)..(16): n is an abasic nucleotide
  misc_feature (20)..(20): n is dT-fluorophore

SEQ ID NO: 96 - DNA, 50 bp, Artificial; identical to SEQ ID NO: 88
  Synthetic
  misc_feature (35)..(35): n is dT-quencher group
  misc_feature (36)..(36): n is an abasic nucleotide
  misc_feature (40)..(40): n is dT-fluorophore

SEQ ID NO: 97 - DNA, 29 bp, Artificial
  Synthetic
  misc_feature (14)..(14): n is dT-quencher group
  misc_feature (16)..(16): n is an abasic nucleotide
  misc_feature (19)..(19): n is dT-fluorophore
ggggaaatggatgngnacnttttggcaaa

SEQ ID NO: 98 - DNA, 50 bp, Artificial; identical to SEQ ID NO: 90
  Synthetic
  misc_feature (35)..(35): n is dT-quencher group
  misc_feature (37)..(37): n is an abasic nucleotide
  misc_feature (40)..(40): n is dT-fluorophore

SEQ ID NO: 99 - DNA, 28 bp, Artificial
  Synthetic
  misc_feature (13)..(13): n is dT-quencher group
  misc_feature (16)..(16): n is an abasic nucleotide
  misc_feature (18)..(18): n is dT-fluorophore
gggaaatggatgnggncnttttggcaaa

SEQ ID NO: 100 - DNA, 50 bp, Artificial; identical to SEQ ID NO: 92
  Synthetic
  misc_feature (35)..(35): n is dT-quencher group
  misc_feature (38)..(38): n is an abasic nucleotide
  misc_feature (40)..(40): n is dT-fluorophore

SEQ ID NO: 101 - DNA, 27 bp, Artificial
  Synthetic
  misc_feature (12)..(12): n is dT-quencher group
  misc_feature (16)..(16): n is an abasic nucleotide
  misc_feature (17)..(17): n is dT-fluorophore
ggaaatggatgnggannttttggcaaa

SEQ ID NO: 102 - DNA, 50 bp, Artificial; identical to SEQ ID NO: 94
  Synthetic
  misc_feature (35)..(35): n is dT-quencher group
  misc_feature (39)..(39): n is an abasic nucleotide
  misc_feature (40)..(40): n is dT-fluorophore

SEQ ID NO: 103 - DNA, 50 bp, Artificial
  Synthetic
  misc_feature (50)..(50): wherein the A at position 50 is modified to block polymerase extension
agaacgttagtggacagaggctggggaaatggatgtggactttttggcaa

SEQ ID NO: 104 - DNA, 50 bp, Artificial
  Synthetic
  misc_feature (50)..(50): wherein the A at position 50 is modified to block polymerase extension
aagaacgttagtggacagaggctggggaaatggatgtggactttttggca

SEQ ID NO: 105 - DNA, 50 bp, Artificial
  Synthetic
  misc_feature (50)..(50): wherein the C at position 50 is modified to block polymerase extension
aaagaacgttagtggacagaggctggggaaatggatgtggactttttggc

SEQ ID NO: 106 - DNA, 50 bp, Artificial
  Synthetic
  misc_feature (50)..(50): wherein the G at position 50 is modified to block polymerase extension
aaaagaacgttagtggacagaggctggggaaatggatgtggactttttgg

SEQ ID NO: 107 - DNA, 50 bp, Artificial
  Synthetic
  misc_feature (50)..(50): wherein the G at position 50 is modified to block polymerase extension
caaaagaacgttagtggacagaggctggggaaatggatgtggactttttg

SEQ ID NO: 108 - DNA, 50 bp, Artificial
  Synthetic
  misc_feature (50)..(50): wherein the T at position 50 is modified to block polymerase extension
gcaaaagaacgttagtggacagaggctggggaaatggatgtggacttttt

SEQ ID NO: 109 - DNA, 50 bp, Artificial
  Synthetic
  misc_feature (50)..(50): wherein the G at position 50 is modified to block polymerase extension
aacgttagtggacagaggctggggaaatggatgtggactttttggcaaag

SEQ ID NO: 110 - DNA, 50 bp, Artificial
  Synthetic
  misc_feature (50)..(50): wherein the G at position 50 is modified to block polymerase extension
acgttagtggacagaggctggggaaatggatgtggactttttggcaaagg

SEQ ID NO: 111 - DNA, 50 bp, Artificial
  Synthetic
  misc_feature (50)..(50): wherein the G at position 50 is modified to block polymerase extension
cgttagtggacagaggctggggaaatggatgtggactttttggcaaaggg

SEQ ID NO: 112 - DNA, 50 bp, Artificial
  Synthetic
  misc_feature (50)..(50): wherein the A at position 50 is modified to block polymerase extension
gttagtggacagaggctggggaaatggatgtggactttttggcaaaggga

SEQ ID NO: 113 - DNA, 50 bp, Artificial
  Synthetic
  misc_feature (50)..(50): wherein the G at position 50 is modified to block polymerase extension
ttagtggacagaggctggggaaatggatgtggactttttggcaaagggag

SEQ ID NO: 114 - DNA, 50 bp, Artificial
  Synthetic
  misc_feature (50)..(50): wherein the C at position 50 is modified to block polymerase extension
tagtggacagaggctggggaaatggatgtggactttttggcaaagggagc

SEQ ID NO: 115 - DNA, 23 bp, Artificial
  Synthetic
ttggtcatgatactgctgattgc

SEQ ID NO: 116 - DNA, 22 bp, Artificial
  Synthetic
ccttccacaaagtccctattgc

SEQ ID NO: 117 - DNA, 17 bp, Artificial
  Synthetic
ccgctgcccaacacaag

SEQ ID NO: 118 - DNA, 24 bp, Artificial
  Synthetic
ccactaacgttcttttgcagacat

SEQ ID NO: 119 - DNA, 27 bp, Artificial
  Synthetic
  misc_feature (1)..(1): wherein the C at position 1 is modified with a FAM Label
cggcatacagcatcaggtgcataggag

SEQ ID NO: 120 - DNA, 31 bp, Artificial
  Synthetic
  misc_feature (1)..(1): wherein the A at position 1 is modified with a FAM Label
agcctaccttgacaagcagtcagacactcaa